SEQUENCE LISTING (73 sequences)

SEQ ID NO: 1 - protein, 579 aa, Artificial Sequence
  Description of Artificial Sequence Synthetic polypeptide
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYSCGWFGGSIPAACITYGCYFIKSNWSWRIPLILQAFTCLIVMSSVFFLPESPRYLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 2 - protein, 579 aa, Artificial Sequence
  Description of Artificial Sequence Synthetic polypeptide
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYSCGWFGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 3 - protein, 579 aa, Artificial Sequence
  Description of Artificial Sequence Synthetic polypeptide
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFLPESPRYLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 4 - protein, 579 aa, Neurospora crassa
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 5 - protein, 918 aa, Saccharomyces cerevisiae [sequence omitted]

SEQ ID NO: 6 - protein, 884 aa, Saccharomyces cerevisiae
MDPNSNSSSETLRQEKQGFLDKALQRVKGIALRRNNSNKDHTTDDTTGSIRTPTSLQRQNSDRQSNMTSVFTDDISTIDDNSILFSEPPQKQSMMMSICVGVFVAVGGFLFGYDTGLINSITSMNYVKSHVAPNHDSFTAQQMSILVSFLSLGTFFGALTAPFISDSYGRKPTIIFSTIFIFSIGNSLQVGAGGITLLIVGRVISGIGIGAISAVVPLYQAEATHKSLRGAIISTYQWAITWGLLVSSAVSQGTHARNDASSYRIPIGLQYVWSSFLAIGMFFLPESPRYYVLKDKLDEAAKSLSFLRGVPVHDSGLLEELVEIKATYDYEASFGSSNFIDCFISSKSRPKQTLRMFTGIALQAFQQFSGINFIFYYGVNFFNKTGVSNSYLVSFITYAVNVVFNVPGLFFVEFFGRRKVLVVGGVIMTIANFIVAIVGCSLKTVAAAKVMIAFICLFIAAFSATWGGVVWVISAELYPLGVRSKCTAICAAANWLVNFICALITPYIVDTGSHTSSLGAKIFFIWGSLNAMGVIVVYLTVYETKGLTLEEIDELYIKSSTGVVSPKFNKDIRERALKFQYDPLQRLEDGKNTFVAKRNNFDDETPRNDFRNTISGEIDHSPNQKEVHSIPERVDIPTSTEILESPNKSSGMTVPVSPSLQDVPIPQTTEPAEIRTKYVDLGNGLGLNTYNRGPPSLSSDSSEDYTEDEIGGPSSQGDQSNRSTMNDINDYMARLIHSTSTASNTTDKFSGNQSTLRYHTASSHSDTTEEDSNLMDLGNGLALNAYNRGPPSILMNSSDEEANGGETSDNLNTAQDLAGMKERMAQFAQSYIDKRGGLEPETQSNILSTSLSVMADTNEHNNEILHSSEENATNQPVNENNDLK

SEQ ID NO: 7 - protein, 763 aa, Saccharomyces cerevisiae
MNDSQNCLRQREENSHLNPGNDFGHHQGAECTINHNNMPHRNAYTESTNDTEAKSIVMCDDPNAYQISYTNNEPAGDGAIETTSILLSQPLPLRSNVMSVLVGIFVAVGGFLFGYDTGLINSITDMPYVKTYIAPNHSYFTTSQIAILVSFLSLGTFFGALIAPYISDSYGRKPTIMFSTAVIFSIGNSLQVASGGLVLLIVGRVISGIGIGIISAVVPLYQAEAAQKNLRGAIISSYQWAITIGLLVSSAVSQGTHSKNGPSSYRIPIGLQYVWSSILAVGMIFLPESPRYYVLKDELNKAAKSLSFLRGLPIEDPRLLEELVEIKATYDYEASFGPSTLLDCFKTSENRPKQILRIFTGIAIQAFQQASGINFIFYYGVNFFNNTGVDNSYLVSFISYAVNVAFSIPGMYLVDRIGRRPVLLAGGVIMAIANLVIAIVGVSEGKTVVASKIMIAFICLFIAAFSATWGGVVWVVSAELYPLGVRSKCTAICAAANWLVNFTCALITPYIVDVGSHTSSMGPKIFFIWGGLNVVAVIVVYFAVYETRGLTLEEIDELFRKAPNSVISSKWNKKIRKRCLAFPISQQIEMKTNIKNAGKLDNNNSPIVQDDSHNIIDVDGFLENQIQSNDHMIAADKGSGSLVNIIDTAPLTSTEFKPVEHPPVNYVDLGNGLGLNTYNRGPPSIISDSTDEFYEENDSSYYNNNTERNGANSVNTYMAQLINSSSTTSNDTSFSPSHNSNARTSSNWTSDLASKHSQYTSPQ

SEQ ID NO: 8 - protein, 961 aa, Saccharomyces cerevisiae
MITEGFPPNLNALKGSSLLEKRVDSLRQLNTTTVNQLLGLPGMTSTFTAPQLLQLRIIAITASAVSLIAGCLGMFFLSKMDKRRKVFRHDLIAFLIICDFLKAFILMIYPMIILINNSVYATPAFFNTLGWFTAFAIEGADMAIMIFAIHFAILIFKPNWKWRNKRSGNMEGGLYKKRSYIWPITALVPAILASLAFINYNKLNDDSDTTIILDNNNYNFPDSPRQGGYKPWSAWCYLPPKPYWYKIVLSWGPRYFIIIFIFAVYLSIYIFITSESKRIKAQIGDFNHNVLEEEKEKKKLFGLGHWGKAKWYFRSYFKLPLLHLLRNLKNFFTISFIDPNEETDDSGSSNGTFNFGESSNEIPTLFRKTNTGSDENVSASGGVRLLDYNSAKPLDMSKYAMSEQPDLERNNPFDCENDITLNPSELVSKQKEHKVTFSVENEGLDTRKSSMLGHQTFSCQNSLESPLAMYDNKNDNSDITSNIKEKGGIINNNSNNDDDDNNNNNDNDNDNNNSNNNNNNNNNNNNNNNNNNNNNNNNNNNNNNSNNIKNNVDNNNTNPADNIPTLSNEAFTPSQQFSQERVNNNADRCENSSFTNVQQHFQAQTYKQMKKRRAQIQKNLRAIFIYPLSYIGIWLFPIIADALQYNHEIKHGPTMWVTYIDTCVRPLSCLVDVIVYLFKEKPWNYSWAKTESKYLIEKYILKGELGEKEILKFCHSNWGKRGWYYRGKWKKRKCWKYSTNPLKRILWFVERFFKQLFELKLHFSFYDNCDDFEYWENYYSAKDSNDNKRTESDETKTNSSDRSLPSNSLELQAMLNNITAEEVEVPLFWRIIHHIPMLGGIDLDELNRLLKIRYNNDHFSLPGLKFALNQNKSHDKHQDVSTNSMVKSSFFSSNIVTNDDENSIEEDKNLRYSDASASENYLVKPTIPGTTPDPIIEAQNDNDSSDSSGIDLIAFLRNGPL

SEQ ID NO: 9 - protein, 525 aa, Neurospora crassa
MGIFNKKPVAQAVDLNQIQEEAPQFERVDWKKDPGLRKLYFYAFILCIASATTGYDGMFFNSVQNFETWIKYFGDPRGSELGLLGALYQIGSIGSIPFVPLLTDNFGRKTPIIIGCVIMIVGAVLQATAKNLDTFMGGRTMLGFGNSLAQIASPMLLTELAHPQHRARLTTIYNCLWNVGALVVSWLAFGTNYINNDWSWRIPALLQAFPSIIQLLGIWWVPESPRFLIAKDKHDEALHILAKYHANGDPNHPTVQFEFREIKETIRLEMESTKNSSYLDFFKSRGNRYRLAILLSLGFFSQWSGNAIISNYSSKLYETAGVTDSTAKLGLSAGQTGLALIVSVTMALLVDKLGRRLAFLASTGGMCGTFVIWTLTAGLYGEHRLKGADKAMIFFIWVFGIFYSLAWSGLLVGYAIEILPYRLRGKGLMVMNMSVQCALTLNTYANPVAFDYFGPDHSWKLYLIYTCWIAAEFVFVFFMYVETKGPTLEELAKVIDGDEADVAHIDIHQVEKEVEIHEHEGKSVA

SEQ ID NO: 10 - protein, 579 aa, Neurospora crassa
MSSHGSHDGASTEKHLATHDIAPTHDAIKIVPKGHGQTATKPGAQEKEVRNAALFAAIKESNIKPWSKESIHLYFAIFVAFCCACANGYDASLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 11 - protein, 579 aa, Neurospora crassa
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWAGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 12 - protein, 579 aa, Neurospora crassa
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFVPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 13 - protein, 579 aa, Neurospora crassa
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIAGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 14 - protein, 579 aa, Neurospora crassa
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLAATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 15 - protein, 579 aa, Neurospora crassa
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYSCGWFGGSIPAACITYGCYFIKSNWSWRIPLILQAFTCLIVMSSVFFLPESPRWLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 16 - DNA, 1740 bp, Artificial Sequence
  Description of Artificial Sequence Synthetic polynucleotide
atgtcctctcatggttctcatgatggtgcttctactgaaaaacatttggccactcatgatattgctccaactcatgatgctatcaagatcgtgcccaagggccatggccagacagccacaaagcccggcgcccaagagaaggaggtccgcaacgccgccctatttgcggccatcaaggagtccaatatcaagccctggagcaaggagtccatccacctctatttcgccatcttcgtcgccttttgttgtgcatgcgccaacggttacgatggttcactcatgaccggaatcatcgctatggacaagttccagaaccaattccacactggtgacactggtcctaaagtctctgtcatcttttctctctataccgtgggtgccatggttggagctcccttcgctgctatcctctctgatcgttttggccgtaagaagggcatgttcatcggtggtatctttatcattgtcggctccattattgttgctagctcctccaagctcgctcagtttgtcgttggccgcttcgttcttggcctcggtatcgccatcatgaccgttgctgccccggcctactccatcgaaatcgcccctcctcactggcgcggccgctgcactggcttctacagctgcggttggttcggaggttcgattcctgccgcctgcatcacctatggctgctacttcattaagagcaactggtcatggcgtatccccttgatccttcaggctttcacgtgccttatcgtcatgtcctccgtcttcttcctcccagaatcccctcgctacctatttgccaacggccgcgacgctgaggctgttgcctttcttgtcaagtatcacggcaacggcgatcccaattccaagctggtgttgctcgagactgaggagatgagggacggtatcaggaccgacggtgtcgacaaggtctggtgggattaccgcccgctcttcatgacccacagcggccgctggcgcatggcccaggtgctcatgatctccatctttggccagttctccggcaacggtctcggttacttcaataccgtcatcttcaagaacattggtgtcaccagcacctcccaacagctcgcctacaacatcctcaactccgtcatctccgctatcggtgccttgaccgccgtctccatgactgatcgtatgccccgccgcgcggtgctcattatcggtaccttcatgtgcgccgctgctcttgccaccaactcgggtctttcggctactctcgacaagcagactcaaagaggcacgcaaatcaacctgaaccagggtatgaacgagcaggatgccaaggacaacgcctacctccacgtcgacagcaactacgccaagggtgccctggccgcttacttcctcttcaacgtcatcttctccttcacctacactcccctccagggtgttattcccaccgaggctctcgagaccaccatccgtggcaagggtcttgccctttccggcttcattgtcaacgccatgggcttcatcaaccagttcgctggccccatcgctctccacaacattggctacaagtacatctttgtctttgtcggctgggatcttatcgagaccgtcgcttggtacttctttggtgtcgaatcccaaggccgtaccctcgagcagctcgaatgggtctacgaccagcccaaccccgtcaaggcctccctaaaagtcgaaaaggtcgtcgtccaggccgacggccatgtgtccgaagctatcgttgcttaa

SEQ ID NO: 17 - protein, 362 aa, Fistularia solaris
MSSERKCALITGITGQDGSYLTELLLEKGYEVHGIVRRSSCFNTGRIDHLYKDRHETGVKLFLHYGDLCDATNLISIISNVKPTEVYNLGAMSHVKVSFDMPEYTADCDGVGVLRMLDAIRAAGMEKTVKFYQASTSELYGKVQEVPQSETTPFYPRSPYAVAKQYAFWILVNYREAYGMHLTNGILFNHESPRRGRTFVTRKITCGVAAIHHGKQKTLFLGNLDAKRDWGHARDYVEGMWRMLQQETSDDYVLATGETHTVREFVEKAFAVVNTTVQWQGEKGTVDEVGVDAADPSRILVRIDPRYFRPTEVDLLLGNPAKAKEKLGWSSSTPFDALVKEMVEADLAILRGEMADADNTFD

SEQ ID NO: 18 - protein, 366 aa, Cladosiphon okamuranus
MAEPETKKTKVDEGTVKKAIITGITGQDGSYLAEFLLEKGYEVHGIIRRSSSFNTQRIDHIYRDRHESAVRLKLHYGDLTDSTNLMHIIYEVQPDEIYNLGAMSHVKVSFEMSEYTAEADGVGVLRLLNAIRSAGLEKKTRLYQASTSELYGKVQEIPQKETTPFYPRSPYGVAKQFGYWMLINYREAYGMHLTNGILFNHESPRRGPTFVTRKITRAVARIHRGKQKCIYLGNLDAKRDWGHAKDYIKGMWLMVQRDEPSDYVLSTGECHSVKEFVQEAFAYVGIDITWVGEGVEEYGHVKGDPENVLVRVDPRYFRPTEVELLLGDCTKAKKELGWVPEITFKELVKDMMQADIANVDAGNDHT

SEQ ID NO: 19 - protein, 360 aa, Cladosiphon okamuranus
MQKTALITGITGQDGAYLAELLLEKGYTVHGIKRRSSSFNTGRIDHLYQDPHDRDVKLHLHYGDMTDSTNLIRIMQETQPDEVYNLAAQSHVQVSFETPEYTGNADALGTLRLLEAIRLLGLSEKTRFYQASTSELYGKVQEVPQSETTPFYPRSPYAAAKLYAYWIVVNYREAYGMHASNGILFNHESPIRGETFVTRKITRAAAAIKLGLQDKLYLGNLDAERDWGHAKDYVRGMWLMLQQDKADDYVLATGEKHSVREFVEQAFAELEINIRWEGRGLDEQGFDTKTEKAVVAVDPRYFRPTEVDLLLGSPKKARKALGWAPTTPFRDMIKQMVRSDLNSVSEDSKKGSQASWIKTG

SEQ ID NO: 20 - protein, 271 aa, Cladosiphon okamuranus
MEATKAIFEKYKPTHVIHLAARVGGLFSNLKYKVEFFRENILINDNVMECCRIYKVAKLVSCLSTCIFPDKTTYPIDETMVHNGPPHTSNEGYAYAKRMIDVLNRCYKDEYGCNFTSVIPTNIYGKGDNFSIDNGHVLPGLIHKCYKAKQAGEDLHVWGTGSPLRQFIYNVDLGALMVWTMRNYHEVDPIILSVGEEDEVSIADAAKMIASAMDFEGNVVFDTDKSDGQFKKTACNDLLKKKNPDFKFTSMQDGLKAACDWFCENFETARK

SEQ ID NO: 21 - protein, 311 aa, Phaeodactylum tricornutum
MVTGGSGLVGAAIREYVEGTGALENESWIYLNSKEGDLRNRADTEKIFAKYQPTHVIHLAAKVGGLFANMAQKVEFFRENILINDNIMECSRIYKVEKLVSFLSTCIFPDKTTYPIDETMLHDGPPHPSNEGYAYAKRLIDTMNRAYAEEYGCNFTSIIPTNIYGPHDNFSIQNGHVIPGLIHKCYLAKKDNTPFTIWGSGTPLRQFVYSRDLAELTVWVMREYHDPTPITLSVDEEEEVSIKDVALAVAKAMQFDGQIVFDTSKADGQFKKTACNKKLRSLKADYEFTSMPDGIQQSVDWFVANYDSCRK

SEQ ID NO: 22 - protein, 327 aa, Saccharina japonica
MAETSGTDAAPKKVVMVTGGTGLVGCGIKEFVESDAEAKEKEEYIFLSSKDGDIRNMEETKLIFEKYKPTHVIHLAARVGGLFSNLKYKVEFFRENILINDNVMECCRIYKVEKLVSCLSTCIFPDKTTYPIDETMVHNGPPHVSNEGYAYAKRMIDVLNRCYKEEYGCNFTSVIPTNIYGKGDNFSIDNGHVLPGLIHKCYKAKQAGEDLHVWGTGSPLRQFIYNVDLGALMIWTMRNYHEVDPIILSVGEEDEVSIADAAKMIASAMDFEGNVVFDTDKSDGQFKKTACNDLLKQKNPDFKFTPMKEGLKQACEWFCENYETARK

SEQ ID NO: 23 - protein, 323 aa, Mucor circinelloides
MATESVILVTGGSGLVGEAVKWVIENDKSERYGKKENEKWVFLSSKDGDLRKEQDVKAIFEKYKPTHVIHLAAMVGGLFKNMKYKLDFLRENMLMNDNILWQSKEYNVKKVVSCLSTCIFPDKTTYPIDETMVHNGPPHESNFGYAHGKRMIDVYNHAYHEQFGCHFTSVIPTNIFGPHDNYDLEGSHVLPGLTHKCYLAKKNNTPFVVWGSGKPLRQFIYSRDLAKLFIWTLREYEEIDPIILSVGEKDEVSIKDVADSIVKAMDFQGEYSFDSTKADGQYKKTASNEKLMKYIPDFEFTPFDVAIKESVEWFVENYDTLRK

SEQ ID NO: 24 - protein, 645 aa, Puniceicoccaceae bacterium
MKKALITGITGQDGSYLAELLLEKGYEVHGIIRRASTFNTRDHYEDPHINGKKFLHYGDLADGVQMVKLLYNLQPDEIYHLGAQSHVRVSFDVPEYTGDVTGLGTLRLEAIREVGLNNKCRFYQASSSEMFGMVQEVPQTEKTPYPRSPGCAKVYAYWLTVNYRESYNLHATNGILFNHESPRRGETFVTRKITRAATRIKMGLQDKLYLGNLDAKRDWGYAKEYVEAMWLMLQQDSGDDYVMATNETHSVKEVQETFAQLDMDWEAFVEYDKRYERPTEVDLIGDPSKAKKQLDWEPKVRFKDLVKIMVEADLEIARKEAAFKAATEQSFRLMNKDAKIYVAGHRGMVGSAVVRALEENGFQSIITRTHAELDLTDQSEVRAFFQSNNIQYAVIAAAKVGGIHANNSYPAEFIYENLAIAQNTIHEAYASGVRLLFLGSTCYPKFAKQPIQEASLLTDALEPTNEAYAIAKIAGLKLCQFYRQYGVLYHSAMPTNLYGRGDNYHPENSHVMPALIRRIHEAKEVGAPEVVVWGTGKPLREFLHSEDAASGIVHLLNIENPPDWVNLGSGREISIGDLAQMISSIIGYDGVLKFDTSKPDGTPRKVTDIQLISDTGWSPQISLEEGVASAYQEFLFELKQGTVRF

SEQ ID NO: 25 - protein, 1082 aa, Cladosiphon okamuranus
MQAEFLLEKGYEVHGVKRRASLFNTQRVDHLYEDPHDSDTRLKLHYGDLTDTSNLTRLLRDIEPDEVYNLGAQSHVAVSFEAPEYTADVDATGTLRLLEAIRFLGLEEKTRFYQASTSELYGKVQEIPQSETTPFHPRSPYAVAKMYAYWITVNYRESYGMYACNGILFNHESPRRGETFVTRKITRGLSNIAMGLEPCLYMGNIDALRDWGHAKDYVRMQWMMLQQDEPEDFVIATGVQYSVREFIRWTARELGMELEFSGTGTDEIARVASITGDRVKALKVGDVVMRIDPRYFRPAEVETLLGNPAKAKAKLGWVPEITAQEMCKRIWVAGHRGMVGGAVVRRLEREDCEVICAARDVVDLTRQQEVQDWMAETRPDAIIMAAAKVGGILANDTRPVDFLLQNLQIETNIVEAAHQVDVERFLFLGSSCIYPKMAPQPIPEDSLLTGPLEPTNEWYAIAKIAGIKLMQAYRKQYGRDWISAMPTNLYGPGDNYDLASSHVLPALLRKFHEAKVAGAKHVELWGSGTPLREFMHCDDLADALVFLLQRYSGHDHVNVGSGSEVSIRELAETIAQVVGYEAEIVFDSSKPDGTPRKLMDSARLHDMGWNNARSLLDGLRDTYARGTVVFKSVADEIRTVDVADYSILPVGVVQWLETDGAGDSYNIASRLDFAPNPDIAVISALRPLSNLTPIQRVFHLGGGNQHILLMRMISSQPEDVHNIPHLGWYMRTGVRVIVISAALSSGGLFAIGWILQSSGHAYGRVLIGGAVFLFPMILAEAVMNLARARGSFFMALLPRDIIWRTLVIAIALGLLLALPTGWSGLQLMLICAGSLMICLLVQIRLAWGLYAGHIPPQTAPDWPNWRAQSLWLWISSLAGNISGNLAVLIISMTLSLEAAGVFFAALRLSMVLALPLNALNIAVAPRFSHLHARQDYNALQTYGLRMTQVIALPTLAALALIVAYGDQALSWFDSEITGGWGALCLLAIGYTLRTCAGASGVMMLMTGHERKAVRIFFQTEGLSLLVLPLAAHFYGIEGAAACLALGVAASSVLSNLHLRRSFRVDPGLHSVLLAPRSDQGIL

SEQ ID NO: 26 - protein, 768 aa, Dictyostelium discoideum
MNDSPIISVVLPFLIKDNDDKSLNYQGINNLIISIDSIIEQTFKEWELILVDDGSNNEILEQLLSKRYSTDNRIKFIINKENKGIVKSLNDAILNHCSPTSKYIARMDSDDISHPTRLQSQLKYLQSNETIDILGCPIKMFNNNKLIEILNNNNNNNNINNNVKELINIINNEESFKFIQHPDKDILMWSMFFNCCIVHPSVIFKRSIFTIEHCYEENNQFPFIEDYLFWLKSLIMKGLNISNIQSSTPLLYLRKHNNSISFKNIEKQKDSTANASCYYLNILFKRFNIDSEIIQNSSLSMKEIIQFFQLSPSSLSKINNISIELFEFAFKYLELIEKSCTKQQPNYSNSIKDAANEKMGELVSLCLSNYPNNQKSSLLWEKWLSRNPTSQLLSLLSNLNVKSSTTIINNNINNNNNNNNNNNNNNNNNNNNNNNNNNNNSILNFISGINSNKINTPKSNNNKFKENGIRIICFSKDRAFQLKEYLRTFFKYLKNDDNGNDKFEIIVDVLFTYSNEKFKNSYQLVIESFPQVNFIKEENFTDQLINLVQKTNKLEYVMFSVDDILYYNEFNLKEYCLSLNSEPLALGFYMKLNKNITYCHTCNQDITIPLNSNTISRTENNFKYLKWNRNDNDCKKDWNYPWDLCSTIYRCNDIDSIINGIVKYYGIRNGINHPNRFEFNGNRPIIQKQIYQNKPYCLCLSDHYSPMSVVTINRVQDVYDNPIYDQTLSLDDLDQLLYSNKSLNDEKYKENSLSLNFKSVHIGELFIS

SEQ ID NO: 27 - protein, 343 aa, Homo sapiens
MLVVQMPFSFPMAHFILFVFTVSTIFHVQQRLAKIQAMWELPVQIPVLASTSKALGPSQLRGMWTINAIGRLGNQMGEYATLYALAKMNGRPAFIPAQMHSTLAPIFRITLPVLHSATASRIPWQNYHLNDWMEEEYRHIPGEYVRFTGYPCSWTFYHHLRQEILQEFTLHDHVREEAQKFLRGLQVNGSRPGTFVGVHVRRGDYVHVMPKVWKGVVADRRYLQQALDWFRARYSSLIFVVTSNGMAWCRENIDTSHGDVVFAGDGIEGSPAKDFALLTQCNHTIMTIGTFGIWAAYLTGGDTIYLANYTLPDSPFLKIFKPEAAFLPEWTGIAADLSPLLKH

SEQ ID NO: 28 - protein, 565 aa, Pisum sativa
MNMLIKRVIAIKNPRGDDNNNNKLSDLETLTDKCTTCPLTLMRVMAFFVVSFMLFSVLFSLSVVLRDPPSDAAISSTTTLFQLNQGLGSDDFDSVELLNDKLLGGLLADGFDEKSCLSRYQSAIFGKGLSGKPSSYLISRLRKYEARHKQCGPYTESYNKTVKELGSGQFSESVDCKYVVWISFSGLGNRILTLVSAFLYALLTDRVLLVDPGVDMTDLFCEPFPDASWFVPPDFPLNSHLNNFNQESNQCHGKILKTKSITNSTVPSFVYLHLAHDYDDHDKLFFCDEEQLFLQNVPLLIMKTDNYFIPSLFLMPSFEQELNDLFPKKEKVFHFLGRYLLHPTNNVWGLVVRYYDAYLAKVDERIGIQIRVFDTDPGPFQHVLDQVLACTLKESILPDVNREQNINSSSGTPKSKAVLITSLSSGYFEKVRDMYWEFPTETGEVVGIYQPSHEGYQQTQKQFHNQKAWAEMYLLSLTDVLVTSSWSTFGYVAQGLGGLKPWILYKPENRTAPNPPCQRAMSMEPCFHAPPFYDCKAKRGTDTGALVPHVRHCEDMSWGLKLVDN

SEQ ID NO: 29 - protein, 292 aa, Rhizobium marinum
MITVKLLGGLGNQMFQFATGRAVARRLGSELLLDISSFEHYDLRRFELEDWAINARVATASELARAGVVPSPPRMLTRISRLLGLAFPATTFRESSFAYDPGILQVTDPVYLDGYFQSERYFSDVAGHLREEFVLRQPTDAKNKAMEALIRDAGPLAVSLHIRRGDYVANAQTAKYHGVCSLDYYSAAVDHIAEQVGGGHYFVFSDDLAWVRENLKITQPMTLVDVNGPDKGAWDMALMTACRHHIIANSSFSWWGAWLNPRPDKIIVAPKRWFAGASHDTTDLVPASWIRL

SEQ ID NO: 30 - protein, 293 aa, Herbaspirillum rubrisubalbicans
MIVSRLIGGLGNQMFQYAVGRALAEHMHTPLLLDVSGFVHYDLRRYELDGFNIKAKPASEEELARLGVKAGVKPSMYERAMRKLGIRREPSILREASFTYDARIETVEAPLYLDGYWQSQRYFAAIRPQLLQEFSLKDSWGSANDALAEQIGLAGDGAVSLHVRRGDYVNNAQTAQYHGVCSLDYYRQAVAYIVARVAAPHFFVFSDDHAWVSANLDTGCPTTFVQTNSPDQGIFDMALMKTCRHHIIANSSFSWWGAWLNANDEKIVVAPQRWFNEASKDTSDLIPAGWVRL

SEQ ID NO: 31 - protein, 291 aa, Citrobacter freundii
MQVNRVYVRPMGGLGNQLFQYAVAYGVARKHSAQLIIDTRFFDNYELHGGFRLYNLNISVSEMTNADLKKFPEWKCKLLSKFPQVTRFFNEYIYDKVGDLNEIKSNDAMLLGYWQNETNFHQYKNELVTIFKPKIISENDNKKAESILATNSVVIHIRRGDYINNPIAYKHHGVCSLNYYKQAINEMKKNTKNIFFYIFSDDIEWCRENITPLFSEYDSFSFVRGETQEVDMWLMSCGKYHIIANSSFSWWGAFLSTNPDQIVIAPTPWFDITQKYTGDPSLPQWIKIDKY

SEQ ID NO: 32 - protein, 301 aa, Lactobacillus helveticus
MLYMRLKGRLGNQLFIYAFARELVYKYNQQVLLYDRKDEKDSMWYSHLDNYPLNTNVHFTSNRRDMKIGNFKSKLRFIYDRVAIRHLPPRERYNFQIRNLKKNEKNSLFLLMDGYAPLPKKINDGTFFDGYFQSPKYFNNIREELIKELNPVHTYSEEEKKFINKIKNTESVCVTIRLGDYINNSTHQVCSKEFYLNAMDKLKKIYPDCTFFIFSDEVDKAQQIFDFKYPVIYDSGKMQDYVSLHVMSMCKHFIISNSSFSWWAQYLSTNPQKIVIAPDKWYAQDVPCDIYEDNWVLMKGK

SEQ ID NO: 33 - protein, 313 aa, Neocallimastix californiae
MKILIFSVSFSFFYLLHLFFILYYIISKASKEIRIVKLCGGLGNQMFQYAYGKSLEHKLQEKVLFDVSWYKYLNKKKNEKLTKREYGLGIFNLKISFPTKKQLKKCNNKTFEKKSYIYDEELLQNKGSSYYVGYFQNEKYFKDIKDNIKKIYTFPKIHDTDKFNQQWINKIKNVKNSVFIHIRRADYIYLDGWVLSMDYYKKAIEYIKKNVENPTFFIFCYQCKDYVEEQFKLDDTIQFIGETNSINNENWKDMVLMKECKYAIIANSSFSWWAAWLGRANEEGIVIAPSPFIKNNDEIICDNWIKINSNNSS

SEQ ID NO: 34 - protein, 314 aa, Gracilariopsis chorda
MGLRERLHSVWFLWFVAFSIIAVGFLSRSVRTPSVPQQLKSTVLVTLSGRLGNQLFQVAASEFITARIKPQKVLFLRNNYSAETDFSQGVFRDLKHVNSVSEACRGLRRNYYSHKRMSCSHVRRNQLKGECLIVEGLFQCPHFANAGSSLVRSLFESSLIASKAEETYRSYAAVSPASPVVAIHIRRGDYTKRFNRNFLEPLPMKYYIRATKFMPKNAIYLVFSDDTAWCKSNLPELFRKIPHSRLIFVKETDASISLALMSLADHFIIANSTFSWWAAFLRRFEKKIVVSPKNWFGDRVTEKNKIYPRKWIRV

SEQ ID NO: 35 - protein, 314 aa, Lactobacillus gasseri
MLYVEMDGRCGNQLFHYAVARYIQLAIGNKEKLCLNFNKIFEKKDENNGWIDYLKDFKTVPYSYYSKSGTILKNESNFIQKIAIGLKAIQIKSLTKKSRQEQADKAEVGQRTLNKLGVYWVREGVNQIYPYKNNKILVSGICESNFIYEIQEQLQKELIPVTPVSSLNKSLLEKIDNCNSVCISVRRGDFFNNKNAKKYGVCSPEYYIRAKKYFDKKRLENTVYFCFSDDIEWCKENLKFTDKNVIFVSQEMPVYETLRLMSRCKHFILSNSTFSWWGQFLSEYKDKIVVSPARWNNDGYDTNLIDKNWILIDA

SEQ ID NO: 36 - protein, 342 aa, Octopus bimaculoides
MLLPCWLYHCYCFYHDVAAVGVFHSAFCVKLLIFFIVFFLGVIIFHYLDILGVIYTINYLVHRQQDDTKVLCPKFVGGLGNQMFQYASLYGIAKSKNMTLLIDAECELNQLFSISAVTLPHVACWFLKTRTDYRPCAFNKDTMNFSADQNYQMQGYLQSWQYFHRAEPALRQIFKFKAAIREKAESILKQAIEVHQKQVRNQALTFIAIHIRRGDITKDNFKTYGYNTASLDYIRRAMQYFSERYHRILFLVCTNDMEWAKRYLHKKNVYFVENQPREVDMALMASCNHTIMTVGSFGWWSAWLANGEVVYYRYPASRGSKLRKAFSKEMTDYYYPKWKPML

SEQ ID NO: 37 - protein, 293 aa, Chryseobacterium scophthalmum
MVAVELIGGLGNQMFQYATARALSLHRDDSLLLDSRLFDNYKLHSYCLNHFNIGAAVVKNDLSLKTPGFSKRVVDKLLQKIDALTLQNKIFNTYQEKNLLFDDSLFRNSKKNIYLKGYFQSEKYFAKYEDQLRKDFEIVTPLKKETTDLLKIIEAENSVSLHIRRGDYISNPAANAVHGTCDLNYYHRAIEIIKEKIEHPIFFIFSDDIDWAKENLKLENTTYFVDFNDASTNYEDLKLMSACKNNITANSSFSWWGAWLNANKSKIVIAPSKWFNTDVLNSQDIIPESWMKI

SEQ ID NO: 38 - protein, 313 aa, Homo sapiens
MLAKIQAMWELPVQIPVLASTSKALGPSQLRGMWTINAIGRLGNQMGEYATLYALAKMNGRPAFIPAQMHSTLAPIFRITLPVLHSATASRIPWQNYHLNDWMEEEYRHIPGEYVRFTGYPCSWTFYHHLRQEILQEFTLHDHVREEAQKFLRGLQVNGSRPGTFVGVHVRRGDYVHVMPKVWKGVVADRRYLQQALDWFRARYSSLIFVVTSNGMAWCRENIDTSHGDVVFAGDGIEGSPAKDFALLTQCNHTIMTIGTFGIWAAYLTGGDTIYLANYTLPDSPFLKIFKPEAAFLPEWTGIAADLSPLLKH

SEQ ID NO: 39 - protein, 481 aa, Pisum sativa
MGLGSDDFDSVELLNDKLLGGLLADGFDEKSCLSRYQSAIFGKGLSGKPSSYLISRLRKYEARHKQCGPYTESYNKTVKELGSGQFSESVDCKYVVWISFSGLGNRILTLVSAFLYALLTDRVLLVDPGVDMTDLFCEPFPDASWFVPPDFPLNSHLNNFNQESNQCHGKILKTKSITNSTVPSFVYLHLAHDYDDHDKLFFCDEEQLFLQNVPLLIMKTDNYFIPSLFLMPSFEQELNDLFPKKEKVFHFLGRYLLHPTNNVWGLVVRYYDAYLAKVDERIGIQIRVFDTDPGPFQHVLDQVLACTLKESILPDVNREQNINSSSGTPKSKAVLITSLSSGYFEKVRDMYWEFPTETGEVVGIYQPSHEGYQQTQKQFHNQKAWAEMYLLSLTDVLVTSSWSTFGYVAQGLGGLKPWILYKPENRTAPNPPCQRAMSMEPCFHAPPFYDCKAKRGTDTGALVPHVRHCEDMSWGLKLVDN

SEQ ID NO: 40 - protein, 278 aa, Neocallimastix californiae
MKLCGGLGNQMFQYAYGKSLEHKLQEKVLFDVSWYKYLNKKKNEKLTKREYGLGIFNLKISFPTKKQLKKCNNKTFEKKSYIYDEELLQNKGSSYYVGYFQNEKYFKDIKDNIKKIYTFPKIHDTDKFNQQWINKIKNVKNSVFIHIRRADYIYLDGWVLSMDYYKKAIEYIKKNVENPTFFIFCYQCKDYVEEQFKLDDTIQFIGETNSINNENWKDMVLMKECKYAIIANSSFSWWAAWLGRANEEGIVIAPSPFIKNNDEIICDNWIKINSNNSS

SEQ ID NO: 41 - protein, 587 aa, Kluyveromyces lactis
MADHSSSSSSLQKKPINTIEHKDTLGNDRDHKEALNSDNDNTSGLKINGVPIEDAREEVLLPGYLSKQYYKLYGLCFITYLCATMQGYDGALMGSIYTEDAYLKYYHLDINSSSGTGLVFSIFNVGQICGAFFVPLMDWKGRKPAILIGCLGVVIGAIISSLTTTKSALIGGRWFVAFFATIANAAAPTYCAEVAPAHLRGKVAGLYNTLWSVGSIVAAFSTYGTNKNFPNSSKAFKIPLYLQMMFPGLVCIFGWLIPESPRWLVGVGREEEAREFIIKYHLNGDRTHPLLDMEMAEIIESFHGTDLSNPLEMLDVRSLFRTRSDRYRAMLVILMAWFGQFSGNNVCSYYLPTMLRNVGMKSVSLNVLMNGVYSIVTWISSICGAFFIDKIGRREGFLGSISGAALALTGLSICTARYEKTKKKSASNGALVFIYLFGGIFSFAFTPMQSMYSTEVSTNLTRSKAQLLNFVVSGVAQFVNQFATPKAMKNIKYWFYVFYVFFDIFEFIVIYFFFVETKGRSLEELEVVFEAPNPRKASVDQAFLAQVRATLVQRNDVRVANAQNLKEQEPLKSDADHVEKLSEAESV

SEQ ID NO: 42 - protein, 373 aa, Escherichia coli
MSKVALITGVTGQDGSYLAEFLLEKGYEVHGIKRRASSFNTERVDHIYQDPHTCNPKFHLHYGDLSDTSNLTRILREVQPDEVYNLGAMSHVAVSFESPEYTADVDAMGTLRLLEAIRFLGLEKKTRFYQASTSELYGLVQEIPQKETTPFYPRSPYAVAKLYAYWITVNYRESYGMYACNGILFNHESPRRGETFVTRKITRAIANIAQGLESCLYLGNMDSLRDWGHAKDYVKMQWMMLQQEQPEDFVIATGVQYSVRQFVEMAAAQLGIKLRFEGTGVEEKGVVVSVTGHDAPGVKPGDVIIAVDPRYFRPAEVETLLGDPTKAHEKLGWKPEITLREMVSEMVANDLEAAKKHSLLKSHGYDVAIALES

SEQ ID NO: 43 - protein, 321 aa, Escherichia coli
MSKQRVFIAGHRGMVGSAIRRQLEQRGDVELVLRTRDELNLLDSRAVHDFFASERIDQVYLAAAKVGGIVANNTYPADFIYQNMMIESNIIHAAHQNDVNKLLFLGSSCIYPKLAKQPMAESELLQGTLEPTNEPYAIAKIAGIKLCESYNRQYGRDYRSVMPTNLYGPHDNFHPSNSHVIPALLRRFHEATAQNAPDVVVWGSGTPMREFLHVDDMAAASIHVMELAHEVWLENTQPMLSHINVGTGVDCTIRELAQTIAKVVGYKGRVVFDASKPDGTPRKLLDVTRLHQLGWYHEISLEAGLASTYQWFLENQDRFRG

SEQ ID NO: 44 - protein, 323 aa, Arabidopsis thaliana
MAETIGSEVSSMSDKSAKIFVAGHRGLVGSAIVRKLQEQGFTNLVLKTHAELDLTRQADVESFFSQEKPVYVILAAAKVGGIHANNTYPADFIGVNLQIQTNVIHSAYEHGVKKLLFLGSSCIYPKFAPQPIPESALLTASLEPTNEWYAIAKIAGIKTCQAYRIQHGWDAISGMPTNLYGPNDNFHPENSHVLPALMRRFHEAKVNGAEEVVVWGTGSPLREFLHVDDLADACVFLLDRYSGLEHVNIGSGQEVTIRELAELVKEVVGFEGKLGWDCTKPDGTPRKLMDSSKLASLGWTPKVSLRDGLSQTYDWYLKNVCNR

SEQ ID NO: 45 - protein, 310 aa, Helicobacter pylori
MNEIILITGAYGMVGQNTALYFKKNKPDVTLLTPKKSELCLLDKDNVQAYLKEYKPTGIIHCAGRVGGIVANMNDLSTYMVENLLMGLYLFSSALDSGVKKAINLASSCAYPKFAPNPLKESDLLNGSLEPTNEGYALAKLSVMKYCEYVSAEKGVFYKTLVPCNLYGEFDKFEEKIAHMIPGLIARMHTAKLKNEKEFAMWGDGTARREYLNAKDLARFISLAYENIASIPSVMNVGSGVDYSIEEYYEKVAQVLDYKGVFVKDLSKPVGMQQKLMDISKQRALKWELEIPLEQGIKEAYEYYLKLLEV

SEQ ID NO: 46 - protein, 322 aa, Mortierella alpine
MSPSKSVIMVTGGSGLVGKAIDWVVENDSKYGKREGEEWVFLTSKDGNLIDPAQTKAIFEKYRPTHVIHLAAKVGGLFGNMAANLDYFRDNLLINDNVLHNAKEFGVKKVVSCLSTCIFPDKTSYPIDETMVHQGPPHDSNYGYSHSKRMVDVMNRAYNQQYGCNFTSVIPTNVFGPHDNFHLVNSHVIPGLIHKCYLAQQNNTPFIMAGTGRPLRQFIYSRDLARLFIWTLREYQEITPLILSVPEEEEVSIKQVGDSIVKAMGYTGDYRFDTTKADGQYKKTASNKKLMSLNPDFQFTPFDVALSETVEWFKENVDTIRK

SEQ ID NO: 47 - protein, 298 aa, Escherichia coli
MRSIIRLQGGLGNQLFQFSFGYALSKINGTPLYFDISHYAENDDHGGYRLNNLQIPEEYLQYYTPKINNIYKFLVRGSRLYPEIFLFLGFCNEFHAYGYDFEYIAQKWKSKKYIGYWQSEHFFHKHILDLKEFFIPKNVSEQANLLAAKILESQSSLSIHIRRGDYIKNKTATLTHGVCSLEYYKKALNKIRDLAMIRDVFIFSDDIFWCKENIETLLSKKYNIYYSEDLSQEEDLWLMSLANHHIIANSSFSWWGAYLGTSASQIVIYPTPWYDITPKNTYIPIVNHWINVDKHSSC

SEQ ID NO: 48 - protein, 298 aa, Helicobacter pylori
MAFKVVQICGGLGNQMFQYAFAKSLQKHLNTPVLLDITSFDWSNRKMQLELFPIDLPYASAKEIAIAKMQHLPKLVRDTLKCMGFDRVSQEIVFEYEPGLLKPSRLTYFYGYFQDPRYFDAISPLIKQTFTLPPPENGNNKKKEEEYHRKLALILAAKNSVFVHVRRGDYVGIGCQLGIDYQKKALEYIAKRVPNMELFVFCEDLKFTQNLDLGYPFMDMTTRDKEEEAYWDMLLMQSCKHGIIANSTYSWWAAYLINNPEKIIIGPKHWLFGHENILCKEWVKIESHFEVKSKKYNA

SEQ ID NO: 49 - protein, 287 aa, Bacteroides fragilis
MLYVILRGRLGNNLFQIATAASLTQNFIFCTVNKDQERQVLLYKDSFFKNIKVMKGVPDGIPYYKEPFHEFSRIPYEEGKDLIIDGYFQSEKYFKRSVVLDLYRITDELRKKIWNICGNILEKGETVSIHVRRGDYLKLPHALPFCGKSYYKNAIQYIGEDKIFIICSDDIDWCKKNFIGKRYYFIENTTPLLDLYIQSLCTHNIISNSSFSWWGAWLNENSNKIVIAPQMWFGISVKLGVSDLLPVSWVRLPNNYTLGRYCFALYKVVEDYLLNILRLIWKRKKNM

SEQ ID NO: 50 - protein, 290 aa, Escherichia coli
MSIVVARLAGGLGNQMFQYAKGYAESVERNSSLKLDLRGYKNYTLHGGFRLDKLNIDNTFVMSKKEMCIFPNFIVRAINKFPKLSLCSKRFESEQYSKKINGSMKGSVEFIGFWQNERYFLEHKEKLREIFTPININLDAKELSDVIRCTNSVSVHIRRGDYVSNVEALKIHGLCTERYYIDSIRYLKERFNNLVFFVFSDDIEWCKKYKNEIFSRSDDVKFIEGNTQEVDMWLMSNAKYHIIANSSFSWWGAWLKNYDLGITIAPTPWFEREELNSFDPCPEKWVRIEK

SEQ ID NO: 51 - protein, 302 aa, Escherichia coli
MYSCLSGGLGNQMFQYAAAYILQRKLKQRSLVLDDSYFLDCSNRDTRRRFELNQFNICYDRLTTSKEKKEISIIRHVNRYRLPLFVTNSIFGVLLKKNYLPEAKFYEFLNNCKLQVKNGYCLFSYFQDATLIDSHRDMILPLFQINEDLLHLCNDLHIYKKVICENANTTSLHIRRGDYITNPHASKFHGVLPMDYYEKAIRYIEDVQGEQVIIVFSDDVKWAENTFANQPNYYVVNNSECEYSAIDMFLMSKCKNNIIANSTYSWWGAWLNTFEDKIVVSPRKWFAGNNKSKLTMDSWINL

SEQ ID NO: 52 - protein, 283 aa, Escherichia coli
MEVKIIGGLGNQMFQYATAFAIAKRTHQNLTVDISDAVKYKTHPLRLVELSCSSEFVKKAWPFEKYLFSEKIPHFMKKGMFRKHYVEKSLEYDPDIDTKSINKKIVGYFQTEKYFKEFRHELIKEFQPKTKFNSYQNELLNLIKENDTCSLHIRRGDYVSSKIANETHGTCSEKYFERAIDYLMNKGVINKKTLLFIFSDDIKWCRENIFFNNQICFVQGDAYHVELDMLLMSKCKNNIISNSSFSWWAAWLNENKNKTVIAPSKWFKKDIKHDIIPESWVKL

SEQ ID NO: 53 - protein, 301 aa, Escherichia coli
MVMMYCCLSGGLGNQMFQYAAAYILKQHFPDTILVLDDSYYFNQPQKDTIRHLELDQFKIIFDRFSSKDEKVKINRLRKHKKIPLLNSFLQFTAIKLCNKYSLNDASYYNPESIKNIDVACLFSFYQDSKLLNEHRDLILPLFEIRDDLRVLCHNLQIYSLITDSKNITSIHVRRGDYVNNKHAAKFHGTLSMDYYISAMEYIESECGSQTFIIFTDDVIWAKEKFSKYSNCLVADADENKFSVIDMYLMSLCNNNIIANSTYSWWGAWLNRSEDKLVIAPKQWYISGNECSLKNENWIAM

SEQ ID NO: 54 - protein, 477 aa, Helicobacter pylori
MVFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNYDDLRVNYDDLRVNYDDLRVNYDDLRVNYDDLRVNYDDLRVNYDDLRVNYDDLRVNYDDLRVNYDRLLQNASPLLELSQNTTFKIYRKAYQKSLPLLRTIRRWVKK

SEQ ID NO: 55 - protein, 286 aa, Helicobacter mustelae
MDFKIVQVHGGLGNQMFQYAFAKSLQTHLNIPVLLDTTWFDYGNRELGLHLFPIDLQCASAQQIAAAHMQNLPRLVRGALRRMGLGRVSKEIVFEYMPELFEPSRIAYFHGYFQDPRYFEDISPLIKQTFTLPHPTEHAEQYSRKLSQILAAKNSVFVHIRRGDYMRLGWQLDISYQLRAIAYMAKRVQNLELFLFCEDLEFVQNLDLGYPFVDMTTRDGAAHWDMMLMQSCKHGIITNSTYSWWAAYLIKNPEKIIIGPSHWIYGNENILCKDWVKIESQFETKS

SEQ ID NO: 56 - protein, 327 aa, Helicobacter bilis
MEDNLIIVRVDGGIASQIGFVALGKAFEEKGYQVKYDLSWFETSGKGFYNTINGYDRIYDLTFDMPKAFPQLEMKIASEDEVKRYNKLYFIDDEKVITHKPPLYVGGYLGRHYDIYFARHFATYFSPKEIEQKDAPFYILLQEILNTQSCGIHIRRGDLSQNHIVYGEPTSLTYFERVIQLVAQMNSKSVFYLFSDDVAWVREHIAPLLKDKQFKICDINTPEQGYLDLYLLSRCKVIVASHGSLGAYAKILAPHNPLLIAPRVRNVFFEMENVMLVNWGAKLQITQPCNNVITPPPHCQNLTLRYRLFLYLYNRLRSKLLRKGVIQ

SEQ ID NO: 57 - protein, 319 aa, Campylobacter jejuni
MLESNFVIIRVDGGIVSQLYFFAIGKLFEKKGYKVKYDITWFEEEGLGFYNINKGYDKTYNINWDIPKIFPNISIEIASKSEIDQYKKFRVDSELVLEYQPPLYVVGYNSKCDIVEICREIREFFNPLELLSDNKIKFLANEIKRNRSCGVHVRRGDLSQEHVVYGKPTSVDYFFKCINIVRSMYSDAKFYFFSDDNKWVKDNIAPHIENLDYFICDINTPEKGYLDLYFLSLCKIIIGSHGSMGLGAKLLSQEETLFITPKYNYMLFSMSNIMMINFEPKNMEPFNPKIKKIKYKILIKIYYYIRQILLRKFLIKGSD

SEQ ID NO: 58 - protein, 281 aa, Bacteroides vulgatus
MRLIKVTGGLGNQMFIYAFYLRMKKYYPKVRIDLSDMMHYKVHYGYEMHRVFNLPHTEFCINQPLKKVIEFLFFKKIYERKQAPNSLRAFEKKYFWPLLYFKGFYQSERFFADIKDEVRESFTFDKNKANSRSLNMLEILDKDENAVSLHIRRGDYLQPKHWATTGSVCQLPYYQNAIAEMSRRVASPSYYIFSDDIAWVKENLPLQNAVYIDWNTDEDSWQDMMLMSHCKHHIICNSTFSWWGAWLNPNMDKTVIVPSRWFQHSEAPDIYPTGWIKVPVS

SEQ ID NO: 59 - protein, 289 aa, Bacteroides fragilis
MIVSSLRGGLGNQMFIYAMVKAMALRNNVPFAFNLTTDFANDEVYKRKLLLSYFALDLPENKKLTFDFSYGNYYRRLSRNLGCHILHPSYRYICEERPPHFESRLISSKITNAFLEGYWQSEKYFLDYKQEIKEDFVIQKKLEYTSYLELEEIKLLDKNAIMIGVRRYQESDVAPGGVLEDDYYKCAMDIMASKVTSPVFFCFSQDLEWVEKHLAGKYPVRLISKKEDDSGTIDDMFLMMHFRNYIISNSSFYWWGAWLSKYDDKLVIAPGNFINKDSVPESWFKLNVR

SEQ ID NO: 60 - protein, 381 aa, Helicobacter pylori
MKEKIALITGVTGQDGSYLAEYLLNLGYEVHGLKRRSSSINTSRIDHLYEDLHSDHKRRFFLHYGDMTDSSNLIHLIATTKPTEIYNLAAQSHVKVSFETPEYTANADGIGTLRILEAMRILGLEKKTRFYQASTSELYGEVLETPQNENTPFNPRSPYAVAKMYAFYITKNYREAYNLFAVNGILFNHESRVRGETFVTRKITRAASAIAYNLTDCLYLGNLDAKRDWGHAKDYVKMMHLMLQAPIPQDYVIATGKTTSVRDFVKMSFEFIGINLEFQNTGIKEIGLIKSVDEKRANALKLNLSHLKKGQIVVRIDERYFRPTEVDLLLGDPTKAEKELDWVREYDLKELVKDMLEYDLKECQKNLYLQDGGYILRNFYE

SEQ ID NO: 61 - protein, 373 aa, Arabidopsis thaliana
MASENNGSRSDSESITAPKADSTVVEPRKIALITGITGQDGSYLTEFLLGKGYEVHGLIRRSSNFNTQRINHIYIDPHNVNKALMKLHYADLTDASSLRRWIDVIKPDEVYNLAAQSHVAVSFEIPDYTADVVATGALRLLEAVRSHTIDSGRTVKYYQAGSSEMFGSTPPPQSETTPFHPRSPYAASKCAAHWYTVNYREAYGLFACNGILFNHESPRRGENFVTRKITRALGRIKVGLQTKLFLGNLQASRDWGFAGDYVEAMWLMLQQEKPDDYVVATEEGHTVEEFLDVSFGYLGLNWKDYVEIDQRYFRPAEVDNLQGDASKAKEVLGWKPQVGFEKLVKMMVDEDLELAKREKVLVDAGYMDAKQQP

SEQ ID NO: 62 - protein, 365 aa, Mortierella alpine
MSSPIEVVNMSPADYRNRKVALITGITGQDGSYLAELLIEKGYQVHGIIRRSSSFNTGRIEHLYKDAHENPKMRLHHGDLTDSTCLVHIISKVLPTEIYNLGAQSHVKVSFDMSEYTADVDAVGTLRLLDAIRTCGLSHLVRFYQASTSELYGKVAEIPQSETTPFYPRSPYGVAKMYAYWITINYREAYDMYACNGILFNHESPRRGRTFVTRKITCAVASIHLGKQECLYLGNLDAKRDWGHARDYVEGMWRMLQQETAEDFVLATGEMHTVREFVEKSFKAIGSTIRWEGSAEEEVGLDEKGVIRVRVDPAYYRPTEVELLLGNPAKANEKLGWKRQVEFDALVEEMVKSDLIGVAAGDVFN

SEQ ID NO: 63 - protein, 426 aa, Helicobacter pylori
MGFQPLLDAFIESASIEKMASKSPPPPLKIAVANWWGDEEIKEFKKSVLYFILSQRYAITLHQNPNEFSDLVFSNPLGAARKILSYQNTKRVFYTGENESPNFNLFDYAIGFDELDFNDRYLRMPLYYAHLHYKAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHKFSTSFMWEYDLHKPLVSIDDLRVNYDDLRVNYDRLLQNASPLLELSQNTTFKIYRKAYQKSLPLLRAVRKLVKKLGL

SEQ ID NO: 64 - protein, 402 aa, Helicobacter trogontum
MDKQILNMRVLDWWTEDNEQNFYDNIFIRLLQRKYEVVYSDTPDFVLCGPFGYKHLEYRGVRIFCTGENVRPDFNLVDYAISFDYAVFGDRHLRTPLMFLCDDYVEDMQKVLNSRAHLIKSKIKFCSFIASNNYMTEMRDSFFEALCTYKKVDSGGKWKNNIGVYVDDKIEWLKSYKFNICFENDSSPGYLTEKLFDAFMGGCVPIYWGDTSLRCKVDNECGNLIETQEIGYHLNLEQTKKEVDFVYNGGGYGMFDTRIPNIPAYLFDYKINPKAFINAHDFPTFKELIDEIKRIDNDEQAFKDMLNEPVFLNNFNPKEFYSQKTFHFLDYIVSQGPVCAKRIGRGSRLQRKENIMRMFPYDTDSVLIPNFMSYCVKHKKIIDRVRRVCGFPRDIMRTIRGK

SEQ ID NO: 65 - protein, 384 aa, Helicobacter bilis
MQKQQVKMRVLDWWNKDCEENFYNNFFIQILQKKYDVVYSDKPDFILYGPCGYEHLKYDCVRIFYTAENIRPDYNIADYSIDYDYIKFGDRHLRLPYMFWVFCDEMRQKEMDNRISLLDKKEKFCGFMVSNNALTDKRDMFFEALNKYKRVDSGGRWKNNIGGNVDDKIEWLKSYKFNLCFENSSYPGYLTEKLFDAFLAGCVPIYWGDTSLRVHKNTCADSKNSENINNRGGGGNDTFDMRIPNISHSLIDYEINPKAFINAHNFPTFKDLIDEIKRIDNDSYAFESILREPIFLNNFSPYEFYTEQISAFLDHIIMQGANDARRCGDGYWLRTHLEFRRISAKYWNLPSDFLHYCFKYRKIIQGVRDISEYPRNFMRFLRRK

SEQ ID NO: 66 - protein, 340 aa, Helicobacter sp.
MAQNLQTPQDSKTKKRIYFCDGAVKGKIPAILSRHYDIEITPHNPDYVFYSVMGNEHINYDCIRIFSTGENVRADFNFCDYAIGFDYMQFEDRYLRYPFYLHYKEAMEKARNKHLHITPQTLENKKRFCTFVVSNGKADSIRSQFFDKLMQYKHIDSGGKYKNNIGAPVADKLAFLSEGKFNIAFENSSANGYTTEKLIEAFAAGTIPLYWGDESVSLPLDSSGGGVNPKSFVRLNDFASFEEAIAYIEFLDTHNDAYLAILREETFLDSNHEAIFDKKLESFLLHIFNQPLEKAYRRGFGQWRCNIEKRYKKYQRIRSLTNTCVNIIKNPIRRIKKLFK

SEQ ID NO: 67 - protein, 320 aa, Helicobacter hepaticus
MKDDLVILHPDGGIASQIAFVALGLAFEQKGAKVKYDLSWFAEGAKGFWNPSNGYDKVYDITWDISKAFPALHIEIANEEEIERYKSKYLIDNDRVIDYAPPLYCYGYKGRIFHYLYAPFFAQSFAPKEAQDSHTPFAALLQEIESSPSPCGVHIRRGDLSQPHIVYGNPTSNEYFAKSIELMCLLHPQSSFYLFSDDLAFVKEQIVPLLKGKTYRICDVNNPSQGYLDLYLLSRCRNIIGSQGSMGEFAKVLSPHNPLLITPRYRNIFKEVENVMCVNWGESVQHPPLVCSAPPPLVSQLKRNAPLNSRLYKEKDNASA

SEQ ID NO: 68 - protein, 359 aa, Helicobacter hepaticus
MNQGCTKTHKPTKKVYFCDGAVKGKIVALLEQHYELILTNKDPDYIFYSCMGFEHLNYNKVRIFATGENLRADFNFCDYAIGYDYIHFEDRYLRYPLYLHCESDMQKAMNKHLHITPETLQNKSRFCTFVVSNGKADEIRTQFFDFLSQYNRVDSGGRYKNNIGNPVVDKYAFLKEGKFNIAFENSSTNGYITEKLIQAFAAHTIPIYWGDERISLPLDKMGGGINPKSFINMHKYESYKEVLETIYFLDTHDEAYLSMLSEPVFLDKNHQKIFDEKLENFLLHIFNQPLEKAYRRGFGQWRCNIEKRYKKAQKARQIVNNFANIFQIPLRTLKKYLLSIYLSATSKSFVFFTKERTSK

SEQ ID NO: 69 - protein, 331 aa, Bacteroides fragilis
MCDCLSIILLVKMKKIYLKFVDFWDGFDTISNFIVDALSIQYEVVLSNEPDYLFYSCFGTSHLEYDCIKIMFIGENIVPDFNVCDYAIGFNYIDFGDRYLRLPLYAIYDGFSNLQNKKIDVNKALDRKFCSIVVSNNKWADPIRETFFKLLSSYKKVDSGGRAWNNIGGPVDNKLDFISQYKFNIAFENSRVLGYTTEKIMEPMQVNSIPVYWGNPLVGKDFNVDSFVNAHDFDSLERLVEYIIELDSSKDKYLEMLEKPWLLDKTYLDWKQLLLNFINNIMMKSYKDAKYLVNYGHAGKYRNEQRFWGRCERKFKLQRIIEYYSQLFDRK

SEQ ID NO: 70 - protein, 949 aa, Bacteroides fragilis
MQKLLSLPPNLVQSFHELERVNRTDWFCTSDPVGKKLGSGGGTSWLLEECYNEYSDGATFGEWLEKEKRILLHAGGQSRRLPGYAPSGKILTPVPVFRWERGQHLGQNLLSLQLPLYEKIMSLAPDKLHTLIASGDVYIRSEKPLQSIPEADVVCYGLWVDPSLATHHGVFASDRKHPEQLDFMLQKPSLAELESLSKTHLFLMDIGIWLLSDRAVEILIKRSHKESSEELKYYDLYSDFGLALGTHPRIEDEEVNTLSVAILPLPGGEFYHYGTSKELISSTLSVQNKVYDQRRIMHRKVKPNPAMFVQNAVVRIPLCAENADLWIENSHIGPKWKIASRHIITGVPENDWSLAVPAGVCVDVVPMGDKGFVARPYGLDDVFKGDLRDSKTTLTGIPFGEWMSKRGLSYTDLKGRTDDLQAASVFPMVNSVEELGLVLRWMLSEPELEEGKNIWLRSERFSADEISAGANLKRLYAQREEFRKGNWKALAVNHEKSVFYQLDLADAAEDFVRLGLDMPELLPGDALQMSRIHNRMLRARILKLDGKDYRPEEQAAFDLLRDGLLDGISNRKSTPKLDVYSDQIVWGRSPVRIDMAGGWTDTPPYSLYSGGNVVNLAIELNGQPPLQVYVKPCKDFHIVLRSIDMGAMEIVSTFDELQDYKKIGSPFSIPKAALSLAGFAPAFSAVSYASLEEQLKDFGAGIEVTLLAAIPAGSGLGTSSILASTVLGAINDFCGLAWDKNEICQRTLVLEQLLTTGGGWQDQYGGVLQGVKLLQTEAGFAQSPLVRWLPDHLFTHPEYKDCHLLYYTGITRTAKGILAEIVSSMFLNSSLHLNLLSEMKAHALDMNEAIQRGSFVEFGRLVGKTWEQNKALDSGTNPPAVEAIIDLIKDYTLGYKLPGAGGGGYLYMVAKDPQAAVRIRKILTENAPNPRARFVEMTLSDKGFQVSRS

SEQ ID NO: 71 - protein, 981 aa, Bacteroides thetaiotaomicron
MPEPICCFLLCRHSAIAGIQSCYKPINTDSTMQKLLSLPPNLIDSFHQLEEVNHTDWFCTSDPVGSKLGSGGGTTWLLQACHQAFAPEETFSKWIGNEKKILLHAGGQSRRLPGYAPSGKILTPIPVFSWERGQKLGQNLLSLQLPLYERIMKQAPKGLNTLIASGDVYIRSEKPLQDIPEVDVVCYGLWVNPSLATHHGVFVSDRKKPEVLDFMLQKPSLEELEGLSKTHLFLMDIGIWILSDRAVEVLMKRSLKEGTNDISYYDLYSDYGLALGEHPQTTDDEVNKLSVAILPLPGGEFYHFGTSRELISSTLAIQDKVRDQRRIMHRKVKPNPAIFIQNSFTQVKLSAENANLWIENSHVGEGWKLGSRQIITGVPENHWNINLPDGVCIDIVPMGDAAFVARPYGLDDVFKGDLSNDSTTYLGNSFTQWMKEREIGLEDIKGRTDDLQAAPVFPVTTSIEELGILIRWMTAEPQLKEGKELWLRAEKLSADEISAQANLERLYAQRSAFRRDNWKGLSANYEKSVFYQLDLQDAANEFVRLNLDVPAVLKEDAAPMVRIHNRMLRARILKLQGNEGCKGEEQAAFQLLRDGLLEAVAGKKNYPKLNVYSDQIVWGRSPVRIDVAGGWTDTPPYSLYSGGSVVNLAIELNGQPPLQVYVKPCHEFHIVLRSIDMGAVEVIRSYEELQDYKKVGSPFSIPKAALTLAGFAPLFAAESHASLEEHLKAFGSGLEITLLAAIPAGSGLGTSSILASTVLGAINDFCGLAWDRNDICNYTLVLEQLLTTGGGWQDQYGGVFPGVKLLQSESGFEQHPLVRWLPDQLFVQPEYRDCHLLYYTGITRTAKGILAEIVSSMFLNSGKHLSLLAEMKAHAMDMSEAILRGNFETFGNLVGKSWIQNQALDSGTNPPAVAAIIEQIKDYTLGYKLPGAGGGGYLYMVAKDPQAAGCIRRILTEQAPNPRARFVEMTLSDKGLQVSRS

SEQ ID NO: 72 - protein, 950 aa, Bacteroides ovatus
MQKLLSLPPNLIHCFHELEEVNHTDWFCTSDPIGSKLGSGGGTTWLLQACHQAFAPQESFSNWIGHEKRILLHAGGQSRRLPSYGPSGKILTPIPIFSWERGQKLGQNLLSLQLPLYERIMNQAPAGLNTLIASGDVYIRSEKPLQDIPNADVVCYGLWVNPSLATHHGVFVSDRKKPEVLDFMLQKPSLEELEGLSKTHLFLMDIGIWILSDRAIEVLMKRSLKEGTKDITYYDLYSDYGLTLGEHPKTKDEEINQLSVAILPLPGGEFYHYGTSHELISSTLAIQDKVRDQRRIMHRKVKPNPAIFIQNSITQVSLSADNANLWIENSQVGKEWKLGSRQIITGVPENQWSINLPDGVCIDIIPIGENEFVARPYGLDDVFKGALDKITTTYLNVPFTRWMEDRGITWEDIKGRTDDLQSASIFPKVASVEDLGILVRWMTSEPQLEEGKKLWLKAEKVSADEISASANLKRLYEQRNAFRKENWKGLAANYEKSVFYQLDLLDAANEFVRFNLDMPDVLKEDAAPMLRIHNRMLRARIMKLREDKDCAKEEQAAFQLLRDGLLGVMSERKSHPILNVYSDQIVWGRSPVRIDVAGGWTDTPPYSLYSGGSVVNLAIELNGQPPLQVYVKPCKEYHITLRSIDMGAMEVIRNYEELQDYKKVGSPFSIPKAALTLAGFAPAFSTESYPSLAKQLEDFGSGIEITLLAAIPAGSGLGTSSILASTVLGAINDFCGLAWDKNDICSYTLVLEQLLTTGGGWQDQYGGVFSGIKLLQSEAGFEQNPLVRWLPDQFFVHPDYRDCHLLYYTGITRTAKSILAEIVSSMFLNSGPHLSLLAEMKAHAMDMSEAILRSNFESFGRLVGKTWIQNQALDCGTNPPAVAAIIEKIKDYTLGYKLPGAGGGGYLYMVAKDPQAAGQIRRILTEQAPNPRARFVEMTLSDKGLQVSRS

SEQ ID NO: 73 - protein, 5 aa, Unknown
  Description of Unknown wildtype cdt-1 “PEPSR” motif sequence
PESPR

(19) United States
(12) Patent Application Publication
(10) Pub. No.: US 2022/0064686 A1
(43) Pub. Date: Mar. 3, 2022

(54) USE OF SUBSTRATE IMPORTERS FOR THE EXPORT OF OLIGOSACCHARIDES

(71) Applicant: Zimitech, Inc., Oakland, CA (US)

(72) Inventors: James Cate, Berkeley, CA (US); Kulika Chomvong, Pathum Thani (TH); Oliver Kilian, Castro Valley, CA (US); Jingjing Liu, Moraga, CA (US); Jason Liu, Berkeley, CA (US); Yong-Su Jin, Champaign, IL (US)

(21) Appl. No.: 17/282,636

(22) PCT Filed: Oct. 2, 2019
(86) PCT No.: PCT/US2019/054258

§ 371 Date: Apr. 2, 2021

Related U.S. Application Data
(60) Provisional application No. 62/740,049, filed on Oct. 2, 2018.
(60) Provisional application No. 62/801,755, filed on Feb. 6, 2019.

(51) Int. Cl. C12P 19/04; C12N 9/04; C12N 9/10; C12N 9/88

(52) U.S. Cl. CPC C12P 19/04; C12N 9/0006; C12N 9/1051; C12Y 101/01271; C12Y 402/01047; C12Y 204/01069; C12N 9/88

(57) ABSTRACT

Disclosed herein are genetically modified microorganisms and related methods for the enhanced export of oligosaccharides. The microorganisms described herein express major facility superfamily proteins such as CDT-1 which allows for the export of oligosaccharides. Variants of CDT-1 exhibit higher activity regarding oligosaccharide export. Means to export oligosaccharides into the growth medium are provided herein.

### RELATED APPLICATIONS

This application claims the benefit of U.S. Provisional Application No. 62/740,049, filed Oct. 2, 2018, and U.S. Provisional Application No. 62/801,755, filed Feb. 6, 2019. The contents of each of these applications are hereby incorporated by reference in their entirety.

### BACKGROUND

Functional oligosaccharides have emerged as valuable components of food and dietary 7.0 supplements. Their resistance to digestion and fermentation by colonic microbes has given oligosaccharides a nutritional edge. Apart from implications as dietary fibers, sweeteners, and humectants, they are hailed as prebiotics. Their beneficial effects extend from anti-oxidant, anti-inflammatory, immunomodulatory, anti-hypertensive, and anti-allergic to anti-cancer, neuroprotective, and improvement of the skin barrier function and hydration. The rising popularity of bioactive oligosaccharides has accelerated the search for their generation from new, sustainable sources.

Oligosaccharides may be obtained from natural sources and may also be synthesized. Various natural sources of oligosaccharides include milk, honey, sugarcane juice, rye, barley, wheat, soybean, lentils, mustard, fruits, and vegetables such as onion, asparagus, sugar beet, artichoke, chicory, leek, garlic, banana, yacon, tomato, and bamboo shoots. Common oligosaccharide manufacturing methods include hydrolysis of polysaccharides, chemical, and enzymatic polymerization from disaccharide or monosaccharide substrates. Acid, alkali, and enzymatic hydrolysis of polysaccharides can generate oligosaccharides of desired structure and functional properties. In certain cases, enzymatic methods are preferred for oligosaccharide synthesis due to their high selectivity and yields, and environmental-friendly nature. In other cases, oligosaccharide-producing microbial strains may be engineered by introducing exogenous genes to enable oligosaccharide production.

### SUMMARY OF THE INVENTION

Oligosaccharides produced in microorganisms will accumulate intracellularly if not actively transported out of the cell into the medium from where they can be further isolated. Accumulation within the cells in the absence of export processes requires isolation of the oligosaccharide from biomass and limits conversion of the substrate to fermentation product or oligosaccharide. The lack of export of fermentation products out of cells also increases costs of the fermentation processes since fermentation runs effectively have to be stopped once the cells accumulate significant amounts of oligosaccharide in order to recover the latter. In addition, recovery of oligosaccharide from cells require additional processes such as extraction or breakage of cells, or both, which might additionally increase costs and require significant purification steps to remove contaminating cell debris, or both.

Exporter proteins for oligosaccharides are not readily available since organisms typically evolved mechanisms to import, not export, substrates for consumption, sensing or both. The identification of functional substrate transporters allowing for oligosaccharide export which is functional in eukaryotic cells is thus paramount for the production of oligosaccharides in yeasts and other eukaryotic production hosts.

It has been discovered that substrate importers might act as exporters. For example, if oligosaccharides accumulate to high concentrations within cells, this along with the appropriate transporter may drive substrate flow out of the cell where the concentration is lower. Additionally, mutagenized versions of transporters might be impaired in regulation of transport processes in such a way that substrate export along a concentration gradient is facilitated. Additionally, modification of the same substrate transporter can lead to higher fermentation product or oligosaccharide export rates if expressed in an organism accumulating a suitable substrate the cell.

Accordingly, provided herein are transporters that can function as a substrate exporter, particularly for oligosaccharides. Such transporters can also function as importers, and import oligosaccharides, such as an oligosaccharide different from that exported.

CDT-1 (XP_963801.1) from the fungus Neurospora crassa is a substrate transporter from the major facilitator superfamily (MFS) that imports cellobiose into the cell. Unexpectedly, expression of a cellodextrin transporter in an engineered Saccharomyces cerevisiae strain capable of producing a lactose-based oligosaccharide, such as 2′-fucosyllactose (2′-FL), leads to an increase of 2′-FL released into the culture medium. In such circumstances, CDT-1 acts as an exporter facilitating transport of oligosaccharides, such as 2′-FL, out of the cell. Moreover, mutated versions of CDT-1 can act as 2′-FL exporters and in some cases, such mutations further increase 2′-FL export out of the cell, if compared to the non-mutated version of this transporter. CDT-2 is another substrate transporter from the fungus Neurospora crassa that can be used herein for exporting oligosaccharides, such as 2′-FL.

In certain aspects, the present disclosure provides 2′-FL production strains expressing a CDT such as CDT-1, CDT-2 or a CDT mutant (i.e., having one or more alterations in a CDT amino acid sequence).

In one aspect a microorganism comprises a heterologous cellodextrin transporter gene or a construct that enhances expression of the cellodextrin transporter, is provided.

Compared to the parental microorganisms, the microorganisms described herein have an increased ability to produce oligosaccharide products of interest. Accordingly, methods of producing products of interest by culturing the microorganisms of the present disclosure in media containing the oligosaccharides and obtaining the products of interest from the media are provided.

In some embodiments, a CDT mutant is CDT-1SY. These strains show increased export of oligosaccharides if compared to their parental strains not expressing CDT-1 or a CDT-1 analogue.

In certain aspects, the present disclosure provides methods of producing oligosaccharides by culturing the microorganisms disclosed herein. In some embodiments, the microorganisms are bacteria or fungi, for example, filamentous fungi or yeasts. In some embodiments, the microorganisms are yeast, for example, Saccharomyces cerevisiae.

In one aspect a method of producing an oligosaccharide comprising culturing a microorganism described herein in a culture medium and recovering the oligosaccharide is provided herein. In another aspect, a method of isolating an HMO comprising: providing a culture medium with at least one carbon source; providing a microorganism described herein; and culturing the microorganism in the culture medium; wherein a substantial portion of the HMO is exported into the culture medium is provided. In another aspect, a method of isolating an HMO comprising: providing a culture medium with at least one carbon source; providing a microorganism capable of producing and exporting an HMO, wherein the microorganism comprises a heterologous transporter and one or more heterologous HMO production gene(s); and culturing the microorganism in the culture medium; wherein a substantial portion of the HMO is exported into the culture medium is provided.

In another aspect, a product suitable for animal consumption comprising the HMO produced by the microorganism described herein or according to the method described herein and at least one additional ingredient acceptable for animal consumption.

In another aspect, a product suitable for animal consumption comprising the microorganism described herein and optionally at least one additional ingredient acceptable for animal consumption.

### BRIEF DESCRIPTION OF THE DRAWINGS

FIG. 1 shows a schematic of a cell expressing a CDT-1 mutant and a lactose transporter. In this example the cell produces the oligosaccharide 2′-FL. The cell is engineered to produce GDP-fucose. The fucosyl residue in GDP-fucose is subsequently transferred onto lactose, thereby producing 2′-FL. Lactose is imported by a transporter specific for lactose. CDT-1SY facilitates export of oligosaccharides, such as 2′-FL, out of the cell. The oligosaccharide can then be obtained from the growth medium.

FIG. 2 shows the level of 2′-FL in the supernatant in 2′-FL producing background strain either with, or without the transporter CDT-1 mutant (such as CDT-1SY as specified in SEQ ID NO. 1). The strain expressing CDT-1SY exhibits a ˜30% increase in product accumulation in the growth medium.

FIG. 3 shows lactose uptake activity and 2′-FL production by yeast strains expressing CDT-1 M7 (CDT-1 209S 262Y) or Lac12 as lactose transporter along with plasmid based 2′-FL pathway expression consist of GMD, WcaG, and WbgL.

FIG. 4 shows relative lactose uptake activity by yeast strains expressing different CDT-1 mutants. CDT-1 (CDT-1 wild type), M1 (CDT-1 91A), M2 (CDT-1 213A), M3 (CDT-1 256V), M4 (CDT-1 335A), M5 (CDT-1 411A), M6 (CDT-1 209S 262W), M7 (CDT-1 209S 262Y), M8 (CDT-1 209S 262Y first 30 amino acid codons optimized). Ctrl is control strain with no transporter expression,

FIG. 5 shows relative extracellular 2′-FL production by yeast strains expressing different CDT-1 mutants along with plasmid based 2′-FL, pathway expression consist of GMD, WcaG, and WbgL. Ctrl is control strain without any lactose transporter expression.

FIG. 6 shows total 2′-FL production by yeast strains expressing different CDT-1 mutants along with plasmid based 2′-FL pathway expression consist of GMD, WcaG, and WbgL. Ctrl is control strain without any lactose transporter expression.

FIG. 7 shows extracellular 2′-FL ratio by yeast strains expressing different CDT-1 mutants along with plasmid based 2′-FL pathway expression consist of GMD, WcaG, and WbgL.

FIG. 8 shows a schematic of production of fucosylated oligosaccharides within microbes. Shown is an example how the fucosylated oligosaccharide such as 2′-fucosyllactose (2′-FL) is formed. GDP-Mannose is dehydrated to GDP-4-dehydro-6-deoxy-D-mannose by a GDP-dehydratase (GMD). GDP-4-dehydro-6-deoxy-D-mannose is then reduced to GDP-Fucose by a GDP fucose synthase (GFS). In this example, lactose had been imported into the cell by a specific lactose transporter and is then further fucosylated by a glycosyl transferase such as a fucosyl transferase (FT), e.g., alpha-1,2 fucosyltransferase to form 2′-FL. 2′-FL is then exported into the medium by an oligosaccharide transporter.

FIG. 9 shows 2′-FL production by introducing fucosyltransferase (FT) from different organisms to yeast strain with CDT-1 M7, GMD and WcaG expression on plasmids. Ctrl is control strain without FT expression.

FIG. 10 shows relative production of 2′-FL in yeast cells expressing plasmids with GMD, GFS and FT, relative to a base strain that contains a set of genomic GMD, GFS and FT genes. The GFS gene carried on the expression plasmid was here selected from SEQ ID NOs: 20, 21, 22, and 23.

FIG. 11 shows relative production of 2′-FL in yeast cells expressing plasmids with GMD, GFS and FT, relative to a base strain that contains a set of genomic GMD, GFS and FT genes. The FT gene carried on the expression plasmid was selected from SEQ ID NOs: 38, 29, 30, 31, 32, and 40.

FIG. 12 shows relative production of 2′-FL in yeast cells expressing plasmids with (1st column) GMD, a FT and SEQ ID NO: 24 and (2nd column) plasmids with a FT and SEQ ID NO: 24 only, relative to a base strain that contains a set of genomic GMD, GFS and FT genes.

FIG. 13 shows production of 2′-FL by expression of plasmids in a control strain otherwise not capable of 2′-FL production (Ctrl). Strains were transformed with plasmids expressing a GFS and a FT along with a plasmid carrying either SEQ ID NO: 17, 18, or 19, respectively. The control strain carrying no plasmids does not produce any 2′-FL.

### DETAILED DESCRIPTION

In one aspect, a microorganism comprises a heterologous cellodextrin transporter gene or a construct that enhances expression of the cellodextrin transporter, is provided.

Numerous embodiments are further provided that can be applied to any aspect of the present invention described herein. For example, in some embodiments, the heterologous cellodextrin transporter is CDT-1. In some embodiments, the gene or construct that expresses CDT-1 comprises a genetic modification that increases the oligosaccharide export activity of CDT-1 relative to a corresponding wild-type gene or construct that expresses CDT-1. In some embodiments, the gene or construct that expresses CDT-1 is MFS transporter gene (cdt-1) or a variant thereof. In some embodiments, the transporter comprises a PESPR motif. In some embodiments, the CDT-1 has an amino acid sequence of SEQ ID NO: 4 or has at least 60%, 65%, 70%, 75%, 80%, 85%, 90%, 95%, or 99% sequence identity to SEQ ID NO: 4. In some embodiments, one or more amino acid is replaced at positions corresponding to amino acid positions 91, 209, 213, 256, 262, 335, 411 of SEQ ID NO: 4. In some embodiments, the CDT-1 further comprises one or more mutations selected from the group consisting of 91A, 209S, 213A, 256V, 262Y, 335A, and 411A of SEQ ID NO: 4. In some embodiments, the CDT-1 has an amino acid sequence of SEQ ID NO: 4 or has at least 60%, 65%, 70%, 75%, 80%, 85%, 90%, 95%, or 99% sequence identity to SEQ ID NO: 4, and wherein the CDT-1 amino acid sequence comprises a serine at the position corresponding to residue 209 and a tyrosine at the position corresponding to residue 262 of SEQ ID No: In some embodiments, the CDT-1 has the sequence of SEQ ID NO: 1 or has at least 60%, 65%, 70%, 75%, 80%, 85%, 90%, 95%, or 99% sequence identity to SEQ ID NO: 1. In some embodiments, the CDT-1 has an amino acid sequence of SEQ ID NO: 4 or has at least 60%, 65%, 70%, 75%, 80%, 85%, 90%, 95%, or 99% sequence identity to SEQ ID NO: 4, and wherein the CDT-1 amino acid sequence comprises a serine at the position corresponding to residue 209 of SEQ ID NO: 4. In some embodiments, the CDT-1 has the sequence of SEQ ID NO: 2 or at least 60%, 65%, 70%, 75%, 80%, 85%, 90%, 95%, or 99% sequence identity to SEQ ID NO: 2. In some embodiments, the CDT-1 has an amino acid sequence of SEQ ID NO: 4 or has at least 60%, 65%, 70%, 75%, 80%, 85%, 90%, 95%, or 99% sequence identity to SEQ ID NO: 4, and wherein the amino acid sequence comprises a tyrosine at the position corresponding to residue 262 of SEQ ID NO: 4. In some embodiments, CDT-1 has the sequence of SEQ ID NO: 3 or at least 60%, 65%, 70%, 75%, 80%, 85%, 90%, 95%, or 99% sequence identity to SEQ ID NO: 3. In some embodiments, the CDT-1 has an amino acid sequence of SEQ ID NO: 4 or has at least 60%, 65%, 70%, 75%, 80%, 85%, 90%, 95%, or 99% sequence identity to SEQ ID NO: 4, and wherein the amino acid sequence comprises an alanine at the position corresponding to residue 91 of SEQ ID NO: 4. In some embodiments, CDT-1 has the sequence of SEQ ID NO: 10 or at least 60%, 65%, 70%, 75%, 80%, 85%, 90%, 95%, or 99% sequence identity to SEQ ID NO: 10. In some embodiments, the CDT-1 has an amino acid sequence of SEQ ID NO: 4 or has at least 60%, 65%, 70%, 75%, 80%, 85%, 90%, 95%, or 99% sequence identity to SEQ ID NO: 4, and wherein the amino acid sequence comprises an alanine at the position corresponding to residue 213 of SEQ ID NO: 4. In some embodiments, CDT-1 has the sequence of SEQ ID NO: 11 or at least 60%, 65%, 70%, 75%, 80%, 85%, 90%, 95%, or 99% sequence identity to SEQ ID NO: 11. In some embodiments, the CDT-1 has an amino acid sequence of SEQ ID NO: 4 or has at least 60%, 65%, 70%, 75%, 80%, 85%, 90%, 95%, or 99% sequence identity to SEQ ID NO: 4, and wherein the amino acid sequence comprises a valine at the position corresponding to residue 256 of SEQ ID NO: 4. In some embodiments, CDT-1 has the sequence of SEQ ID NO: 12 or at least 60%, 65%, 70%, 75%, 80%, 85%, 90%, 95%, or 99% sequence identity to SEQ ID NO: 12. In some embodiments, the CDT-1 has an amino acid sequence of SEQ ID NO: 4 or has at least 60%, 65%, 70%, 75%, 80%, 85%, 90%, 95%, or 99% sequence identity to SEQ ID NO: 4, and wherein the amino acid sequence comprises an alanine at the position corresponding to residue 335 of SEQ ID NO: 4. In some embodiments, CDT-1 has the sequence of SEQ ID NO: 13 or at least 60%, 65%, 70%, 75%, 80%, 85%, 90%, 95%, or 99% sequence identity to SEQ ID NO: 13. In some embodiments, the CDT-1 has an amino acid sequence of SEQ ID NO: 4 or has at least 60%, 65%, 70%, 75%, 80%, 85%, 90%, 95%, or 99% sequence identity to SEQ ID NO: 4, and wherein the amino acid sequence comprises an alanine at the position corresponding to residue 411 of SEQ ID NO: 4. In some embodiments, CDT-1 has the sequence of SEQ ID NO: 14 or at least 60%, 65%, 70%, 75%, 80%, 85%, 90%, 95%, or 99% sequence identity to SEQ ID NO: 14. In some embodiments, the CDT-1 has an amino acid sequence of SEQ ID NO: 4 or has at least 60%, 65%, 70%, 75%, 80%, 85%, 90%, 95%, or 99% sequence identity to SEQ ID NO: 4, and wherein the CDT-1 amino acid sequence comprises a serine at the position corresponding to residue 209 and a Tryptophan at the position corresponding to residue 262 of SEQ ID No: 4. In some embodiments, the CDT-1 has the sequence of SEQ ID NO: 15 or has at least 60%, 65%, 70%, 75%, 80%, 85%, 90%, 95%, or 99% sequence identity to SEQ ID NO: 15. In some embodiments, the CDT-1 is encoded by a codon optimized nucleic acid. In some embodiments, the nucleic acid is optimized for yeast. In some embodiments, at least 5% of the nucleic acid is codon optimized. In some embodiments, at least 90 nucleotides of the nucleic acid are codon optimized. In some embodiments, the CDT-1 is encoded by the nucleic acid of SEQ ID NO: 16. In some embodiments, the microorganism further comprising a genetic modification that increases the oligosaccharide export activity of CDT-1 selected from: a) a promoter operably linked to the cdt-1 gene b) extrachromosomal genetic material comprising cdt-1; c) one or more copies of cdt-1, wherein said copies are integrated into the genome of the microorganism; d) a modified cdt-1 that encodes a constitutively active CDT-1 compared to unmodified CDT-1; e) a modified cdt-1 that encodes a CDT-1 having increased oligosaccharide export activity compared to unmodified CDT-1; f) extrachromosomal genetic material comprising a modified cdt-1 that encodes a constitutively active CDT-1 or a CDT-1 having increased oligosaccharide export activity compared to the corresponding wild-type CDT-1; or g) one or more copies of cdt-1 or a modified cdt-1 that encode a constitutively active CDT-1 or a CDT-1 having increased oligosaccharide export activity compared to the corresponding wild-type CDT-1, wherein said copies are integrated into the genome of the microorganism. In some embodiments, the promoter operably linked to the cdt-1 gene induces expression of cdt-1 at a higher level than an endogenous promoter. In some embodiments, the promoter is specific for the microorganism in which it induces expression of cdt-1. In some embodiments, the heterologous cellodextrin transporter is CDT-2. In some embodiments, the CDT-2 has an amino acid sequence of SEQ ID NO: 9 or has at least 60%, 65%, 70%, 75%, 80%, 85%, 90%, 95%, or 99% sequence identity to SEQ ID NO: 9. In some embodiments, the microorganism further comprising a gene or a construct that expresses a lactose permease. In some embodiments, the lactose permease is Lac12. In some embodiments, the Lac12 has an amino acid sequence of SEQ ID NO: 41 or has at least 60%, 65%, 70%, 75%, 80%, 85%, 90%, 95%, or 99% sequence identity to SEQ ID NO: 41. In some embodiments, the microorganism further comprising one or more heterologous HMO production gene or a construct that enhances the expression of one or more HMO production protein. In some embodiments, the microorganism comprises the heterologous cellodextrin transporter CDT-1, or variant or mutation of CDT-1 such as described herein and further comprising one or more heterologous HMO production gene or a construct that enhances the expression of one or more HMO production protein. In some embodiments, the one or more HMO production protein is an enzyme capable of converting fucose and ATP to fucose-1-phosphate, an enzyme capable of converting the fucose-1-phosphate and GTP to GDP-fucose, and/or a glucosyl transferase. In some embodiments, the one or more HMO production gene is a GDP-Mannose dehydratase gene or the one or more HMO production protein is a GDP-Mannose dehydratase protein. In some embodiments, the one or more HMO production gene is a GDP-L-fucose synthase gene or the one or more HMO production protein is a GDP-L-fucose synthase protein. In some embodiments, the one or more HMO production gene is a fucosyl transferase gene or the one or more HMO production protein is a fucosyl transferase protein. In some embodiments, the gene or construct that expresses GDP-Mannose dehydratase comprises a genetic modification that increases the oligosaccharide production activity of GDP-Mannose dehydratase relative to a corresponding wild-type gene or construct that expresses GDP-Mannose dehydratase. In some embodiments, the gene or construct that expresses GDP-Mannose dehydratase is GDP-Mannose dehydratase gene (gmd) or a variant thereof. In some embodiments, the GDP-Mannose dehydratase has an amino acid sequence of any one of SEQ ID NOs: 17-19, 42, and 61-63 or has at least 60%, 65%, 70%, 75%, 80%, 85%, 90%, 95%, or 99% sequence identity to any one of SEQ ID NOs: 17-19, 42, and 61-63. In some embodiments, the gene or construct that expresses GDP-L-fucose synthase comprises a genetic modification that increases the oligosaccharide production activity of GDP-L-fucose synthase relative to a corresponding wild-type gene or construct that expresses GDP-L-fucose synthase. In some embodiments, the gene or construct that expresses GDP-L-fucose synthase is GDP-L-fucose synthase gene (gfs) or a variant thereof. In some embodiments, the GDP-L-fucose synthase has an amino acid sequence of any one of SEQ ID NOs: 20-23 or has at least 60%, 65%, 70%, 75%, 80%, 85%, 90%, 95%, or 99% sequence identity to any one of SEQ ID NOs: 20-23. In some embodiments, the gene or construct that expresses GDP-L-fucose synthase is WcaG or a variant thereof. In some embodiments, the WcaG has an amino acid sequence of any one of SEQ ID NOs: 43-45 or has at least 60%, 65%, 70%, 75%, 80%, 85%, 90%, 95%, or 99% sequence identity to any one of SEQ ID NOs: 43-45. In some embodiments, the gene or construct that expresses GDP-L-fucose synthase is GMER or a variant thereof. In some embodiments, the GMER has an amino acid sequence of SEQ ID NO: 46 or has at least 60%, 65%, 70%, 75%, 80%, 85%, 90%, 95%, or 99% sequence identity to any one of SEQ ID NO: 46. In some embodiments. In some embodiments, the gene or construct that expresses fucosyl transferase comprises a genetic modification that increases the oligosaccharide production activity of fucosyl transferase, relative to a corresponding wild-type gene or construct that expresses fucosyl transferase. In some embodiments, the gene or construct that expresses fucosyl transferase is fucosyl transferase gene (ft) or a variant thereof. In some embodiments, the fucosyl transferase is alpha 1,2-fucosyl transferase. In some embodiments, the fucosyl transferase has an amino acid sequence of any one of SEQ ID NOs: 26-40 or has at least 60%, 65%, 70%, 75%, 80%, 85%, 90%, 95%, or 99% sequence identity to any one of SEQ ID NO: 26-40. In some embodiments, the gene or construct that expresses fucosyl transferase is wbgL or a variant thereof. In some embodiments, the wbgL, has an amino acid sequence of SEQ ID NO: 47 or has at least 60%, 65%, 70%, 80%, 80%, 85%, 90%, 95%, or 99% sequence identity of SEQ ID NO: 47. In some embodiments, the gene or construct that expresses fucosyl transferase is futC or a variant thereof. In some embodiments, the futC has an amino acid sequence of SEQ ID NO: 48 or has at least 60%, 65%, 70%, 75%, 80%, 85%, 90%, 95%, or 99% sequence identity of SEQ ID NO: 48. In some embodiments, the gene or construct that expresses fucosyl transferase is wcfB or a variant thereof. In some embodiments, the wcfB has an amino acid sequence of SEQ ID NO: 49 or has at least 60%, 65%, 70%, 75%, 80%, 85%, 90%, 95%, or 99% sequence identity of SEQ ID NO: 49. In some embodiments, the gene or construct that expresses fucosyl transferase is wbgN or a variant thereof. In some embodiments, the wbgN has an amino acid sequence of SEQ ID NO: 50 or has at least 60%, 65%, 70%, 75%, 80%, 85%, 90%, 95%, or 99% sequence identity of SEQ ID NO: 50. In some embodiments, the gene or construct that expresses fucosyl transferase is wbwk or a variant thereof. In some embodiments, the wbwk has an amino acid sequence of any one of SEQ ID NO: 51 or has at least 60%, 65%, 70%, 75%, 80%, 85%, 90%, 95%, or 99% sequence identity to any one of SEQ ID NO: 51. In some embodiments, the gene or construct that expresses fucosyl transferase is wbsJ or a variant thereof. In some embodiments, the wbsJ has an amino acid sequence of SEQ ID NO: 52 or has at least 60%, 65%, 70%, 75%, 80%, 85%, 90%, 95%, or 99% sequence identity of SEQ ID NO: 52. In some embodiments, the gene or construct that expresses fucosyl transferase is wbiQ or a variant thereof. In some embodiments, the wbiQ has an amino acid sequence of SEQ ID NO: 53 or has at least 60%, 65%, 70%, 75%, 80%, 85%, 90%, 95%, or 99% sequence identity of SEQ ID NO: 53. In some embodiments, the gene or construct that expresses fucosyl transferase is futB or a variant thereof. In some embodiments, the futB has an amino acid sequence of SEQ ID NO: 54 or has at least 60%, 65%, 70%, 75%, 80%, 85%, 90%, 95%, or 99% sequence identity of SEQ ID NO: 54. In some embodiments, the gene or construct that expresses fucosyl transferase is futL or a variant thereof. In some embodiments, the futL has an amino acid sequence of SEQ ID NO: 55 or has at least 60%, 65%, 70%, 75%, 80%, 85%, 90%, 95%, or 99% sequence identity of SEQ ID NO: 55. In some embodiments, the gene or construct that expresses fucosyl transferase is futF or a variant thereof. In some embodiments, the futF has an amino acid sequence of SEQ ID NO: 56 or has at least 60%, 65%, 70%, 75%, 80%, 85%, 90%, 95%, or 99% sequence identity of SEQ ID NO: 56. In some embodiments, the gene or construct that expresses fucosyl transferase is futG or a variant thereof. In some embodiments, the futG has an amino acid sequence of SEQ ID NO: 57 or has at least 60%, 65%, 70%, 75%, 80%, 85%, 90%, 95%, or 99% sequence identity of SEQ ID NO: 57. In some embodiments, the gene or construct that expresses fucosyl transferase is futN or a variant thereof. In some embodiments, the futN has an amino acid sequence of SEQ ID NO: 58 or has at least 60%, 65%, 70%, 75%, 80%, 85%, 90%, 95%, or 99% sequence identity of SEQ ID NO: 58. In some embodiments, the gene or construct that expresses fucosyl transferase is wcfw or a variant thereof. In some embodiments, the wcfw has an amino acid sequence of SEQ ID NO: 59 or has at least 60%, 65%, 70%, 75%, 80%, 85%, 90%, 95%, or 99% sequence identity of SEQ ID NO: 59. In some embodiments, the gene or construct that expresses fucosyl transferase is futA or a variant thereof. In some embodiments, the futA has an amino acid sequence of SEQ ID NO: 63 or has at least 60%, 65%, 70%, 75%, 80%, 85%, 90%, 95%, or 99% sequence identity of SEQ ID NO: 63. In some embodiments, the gene or construct that expresses fucosyl transferase is futD or a variant thereof. In some embodiments, the futD has an amino acid sequence of SEQ ID NO: 64 or has at least 60%, 65%, 70%, 75%, 80%, 85%, 90%, 95%, or 99% sequence identity of SEQ ID NO: 64. In some embodiments, the gene or construct that expresses fucosyl transferase is futE or a variant thereof. In some embodiments, the futE has an amino acid sequence of SEQ ID NO: 65 or has at least 60%, 65%, 70%, 75%, 80%, 85%, 90%, 95%, or 99% sequence identity of SEQ ID NO: 65. In some embodiments, the gene or construct that expresses fucosyl transferase is futH or a variant thereof. In some embodiments, the futH has an amino acid sequence of SEQ. ID NO: 66 or has at least 60%, 65%, 70%, 75%, 80%, 85%, 90%, 95%, or 99% sequence identity of SEQ ID NO: 66. In some embodiments, the gene or construct that expresses fucosyl transferase is futJ or a variant thereof. In some embodiments, the futJ has an amino acid sequence of SEQ ID NO: 67 or has at least 60%, 65%, 70%, 75%, 80%, 85%, 90%, 95%, or 99% sequence identity of SEQ ID NO: 67. In some embodiments, the gene or construct that expresses fucosyl transferase is futK or a variant thereof. In some embodiments, the futK has an amino acid sequence of SEQ ID NO: 68 or has at least 60%, 65%, 70%, 75%, 80%, 85%, 90%, 95%, or 99% sequence identity of SEQ ID NO: 68. In some embodiments, the gene or construct that expresses fucosyl transferase is futM or a variant thereof. In some embodiments, the futM has an amino acid sequence of SEQ ID NO: 69 or has at least 60%, 65%, 70%, 75%, 80%, 85%, 90%, 95%, or 99% sequence identity of SEQ ID NO: 69. In some embodiments, the one or more HMO production gene an enzyme comprising two domains, wherein one domain has homology to GDP-Mannose dehydratase and the second domain has homology to fucosyl synthase. In some embodiments, the enzyme has an amino acid sequence of any one of SEQ ID NOs: 24-25 or has at least 60%, 65%, 70%, 75%, 80%, 85%, 90%, 95%, or 99% sequence identity to any one of SEQ ID NO: 24-25. In some embodiments, the one or more HMO production gene is a bifunctional fucokinase-L-fucose-1-P-guanylyltransferase and the one or more HMO production protein is a bifunctional fucokinase/L-fucose-1-P-guanylyltransferase protein. In some embodiments, the bifunctional fucokinase/L-fucose-1-P-guanylyltransferase has an amino acid sequence of any one of SEQ ID NOs: 71-73 or has at least 60%, 65%, 70%, 75%, 80%, 85%, 90%, 95%, or 99% sequence identity to any one of SEQ ID NOs: 71-73. In some embodiments, the microorganism comprises one or more genetic modifications selected from: i) a genetic modification that increases the proton export activity of PMA1 in the microorganism compared to PMA1 activity in the parental microorganism, ii) a genetic modification that decreases the hexose sensing activity of SNF3 in the microorganism compared to SNF3 activity in the parental microorganism, iii) a genetic modification that decreases the hexose sensing activity of RGT2 in the microorganism compared to RGT2 activity in the parental microorganism, and iv) a genetic modification that decreases the hexose sensing activity of GPR1 in the microorganism compared to GPR1 activity in the parental microorganism. In some embodiments, i) the genetic modification that increases the proton export activity of PMA1 is a genetic modification to plasma membrane ATPase gene (pma1), ii) the genetic modification that decreases the hexose sensing activity of SNT3 is a genetic modification to sucrose non-fermenting gene (snf3), the genetic modification that decreases the hexose sensing activity of RGT2 is a genetic modification to restores glucose transport gene (rgt2), and iv) the genetic modification that decreases the hexose sensing activity of GPR1 is a genetic modification to G protein-coupled receptor 1 gene (gpr1). In some embodiments, i) PMA1 has the sequence of SEQ ID NO: 5 or at least 60%, 65%, 70%, 75%, 80%, 85%, 90%, 95%, or 99% sequence identity to SEQ ID NO: 5, ii) SNE3 has the sequence of SEQ ID NO: 6 or at least 60%, 65%, 70%, 75%, 80%, 85%, 90%, 95%, or 99% sequence identity to SEQ ID NO: 6, iii) RGT2 has the sequence of SEQ ID NO: 7 or at least 60%, 65%, 70%, 75%, 80%, 85%, 90%, 95%, or 99% sequence identity to SEQ ID NO: 7, iv) GPR1 has the sequence of SEQ ID NO: 8 or at least 60%, 65%, 70%, 75%, 80%, 85%, 90%, 95%, or 99% sequence identity to SEQ ID NO: 8. In some embodiments, the microorganism further comprises an exogenous nucleotide sequence encoding a chaperonin. In some embodiments, the chaperonin is gGroESL. In some embodiments, the microorganism is a eukaryotic organism In some embodiments, the fungus microorganism is a filamentous fungus or a yeast. In some embodiments, the microorganism is a Ascomycetes fungus. In some embodiments, the Ascomycetes fungus is selected from the group consisting of a Saccharomyces spp., a Schizosaccharomyces spp. and a Pichia spp. In some embodiments, the microorganism is Saccharomyces sp., Saccharomyces cerevisiae, Saccharomyces monacensis, Saccharomyces bayanus, Saccharomyces pastorianus, Saccharomyces carlsbergensis, Saccharomyces pombe, Kluyveromyces sp., Kluyveromyces marxiamus, Kluyveromyces lactis, Kluyveromyces fragilis, Pichia stipitis, Sporotrichum thermophile, Candida shehatae, Candida tropicalis, Neurospora crassa, Neurospora sp, Torulaspora spp., Torulaspora delbrueckii, Zygosaccharomyces spp., Zygosaccharomyces bailii, Brettanomyces spp., Brettanomyces intermedius, Brettanomyces bruxellensis, Brettanomyces anomalus, Brettanomyces custersianus, Brettanomyces naardenensis, Brettanomyces nanus, Dekkera spp., Dekkera bruxellensis, Dekkera anomala, Metschmkowia spp., Issatchenkia spp., Issatchenkia orientails, Issatchenkia terricola, Kloeckera spp., Kloeckera apiculate, Aureobasidium spp., Aureobasidium pullulans, Rhodotorula spp., Rhodotorula glutinis, Rhodotorula cladiensis, Rhodosporidium spp., Rhodosporidum toruloides, Cryptococcus spp., Cryptococcus neoformans, Cryptococcus albidus, Yarrowia spp., Yarrowia lipolytica, Kuraishia spp., Kuraishia capsulata, Kuraishia molischiana, Komagataella spp., Komagataella phaffii, Komagataella pastoris, Hanseniaspora spp., Hanseniaspora guilliermondii, Hanseniaspora uvarum, Hasegawaea spp., Hasegawaea japonica, Ascoidea spp., Ascoidea asiatica, Cephaloascus spp., Cephaloascus fragrans, Lipomyces spp., Lipomyces starkeyi, Kawasakia spp., Kawasakia arxii, Zygozyma spp., Zygozyma oligophaga, Metschnikowia spp., Metschnikowia pulcherrima, Coccidiodes spp., Coccidiodes immitis, Neurospora discretes, Neurospora africana, Aspergillus spp., Aspergillus niger, Aspergillus nidulans, Aspergillus oryzae, Aspergillus fumigatus, Mucor spp., Mucor circinelloides, Mucor racemosus, Rhizopus spp., Rhizopus oryzae, Rhizopus stolonifera, Umbelopsis spp., Umbelapsis Mortierella spp., Mortierella alpine, Alternaria spp., Alternaria alternate, Botrytis spp., Botrytis cinereal, Fusarium spp., Fusarium graminarium, Geotrichum spp., Geotrichum candidum, Penicillium spp., Penicillum chrysogenum, Chaetomium spp., Chaetomium thermophila, Magnaporthe spp., Magnaporthe grisea, Emericella spp., Emericella discophora, Trichoderma spp., Trichodema reesei, Talaromyces spp., Talaromyces emersonii, Sordaria spp., or Sordaria macrospora. In some embodiments, the microorganism has a higher capacity, compared to the parental microorganisms, to transport an oligosaccharide out of the microorganism. In some embodiments, the microorganism has a higher capacity, compared to the parental microorganisms, to transport an oligosaccharide selected from 2-fucosyllactose, 3-fucosyllactose, 6′-fucosyllactose, 3′-sialyllactose, di-fucosyllactose, lacto-N-neotetraose, lacto-N-tetraose, lacto-N-fucopentaose I, lacto-N-fucopentaose II, lacto-N-fucopentaose lacto-N-fucopentaose IV, lacto-N-fucopentaose V, lacto-N-fucopentaose VI, lacto-N-hexaose, lacto-N-neohexaose, monofucosyllacto-N-hexaose I, monofucosyllacto-N-hexaose II, difucosyllacto-N-hexaose I, difucosyllacto-N-hexaose II, difucosyllacto-N-neohexaose, difucosyl-para-lacto-N-neohexaose, difucosyl-para-lacto-N-hexaose, trifucosyllacto-N-hexaose, sialyllacto-N-neotetraose a, sialyllacto-N-tetraose b, sialyllacto-N-tetraose c, disialyllacto-N-tetraose, fucosylsialyllacto-N-tetraose a, fucosylsialyllacto-N-tetraose b, fucosylsialyllacto-N-hexaose, fucosylsialyllacto-N-neohexaose I, or fucosyldisialyllacto-N-hexaose II out of the microorganism. In some embodiments, the microorganism has a higher capacity, compared to the parental microorganisms, to transport a human milk oligosaccharide with a degree of polymerization of 3 out of the organism. In some embodiments, the human milk oligosaccharide is 2-fucosyllactose, 3-fucosyllactose, 6′-fucosyllactose, 3′-sialyllactose, or 6′-sialyllactose. In some embodiments, the microorganism has a higher capacity, compared to the parental microorganisms, to transport a human milk oligosaccharide with a degree of polymerization of 4 out of the organism. In some embodiments, the human milk oligosaccharide is di-fucosyllactose, lacto-N-neotetraose, lacto-N-tetraose, sialyllacto-N-neotetraose a, sialyllacto-N-tetraose b, sialyllacto-N-tetraose c, disialyllacto-N-tetraose, fucosylsialyllacto-N-tetraose a, or fucosylsialyllacto-N-tetraose b. In some embodiments, the microorganism has a higher capacity, compared to the parental microorganisms, to transport a human milk oligosaccharide with a degree of polymerization of 5 out of the organism. In some embodiments, the human milk oligosaccharide is lacto-N-fucopentaose I, lacto-N-fucopentaose II, lacto-N-fucopentaose III, lacto-N-fucopentaose IV, lacto-N-fucopentaose V, lacto-N-fucopentaose VI. In some embodiments, the microorganism has a higher capacity, compared to the parental microorganisms, to transport 2′-fucosyllactose out of the organism. In some embodiments, the microorganism has a higher capacity, compared to the parental microorganisms, to transport lacto-N-tetraose out of the organism. In some embodiments, the microorganism has a higher capacity, compared to the parental microorganisms, to transport lacto-N-neotetraose out of the organism. In some embodiments, the microorganism has a higher capacity, compared to the parental microorganisms, to transport 3′-sialyllactose out of the organism. In some embodiments, the microorganism has a higher capacity, compared to the parental microorganisms, to transport 6′-sialyllactose out of the organism. In some embodiments, the microorganism has a higher capacity, compared to the parental microorganisms, to transport di-fucosyllactose out of the organism. In some embodiments, the microorganism has a higher capacity, compared to the parental microorganisms, to transport lacto-N-fucopentaose I out of the organism.

In another aspect, a microorganism for enhanced production of a human milk oligosaccharide (HMO) comprising a heterologous CDT-1 transporter or a variant thereof and at least one heterologous pathway gene for production of the HMO, is provided.

As described above, certain embodiments are applicable to any microorganism described herein. For example, in some embodiments, the microorganism is capable of producing and exporting the HMO. In some embodiments, the transporter is capable of exporting at least 20%, 30%, 40%, 50%, or 60% of the produced HMO. In some embodiments, the microorganism is capable of exporting at least 50% more of the HMO than a parental microorganism lacking the transporter. In some embodiments, the yeast comprises a transporter that has an amino sequence of SEQ ID NO:4 or a sequence with at least 80%, 85%, 90%, 95%, 98% or 99% homology thereto. In some embodiments, the transporter comprises a PESPR motif. In some embodiments, the transporter comprises a sequence having one or more amino acid replacements at positions corresponding to amino acid positions 91, 209, 213, 256, 262, 335, 411 of SEQ ID NO:4. In some embodiments, the CDT-1 is encoded by a codon optimized nucleic acid. In some embodiments, at least the first 90 nucleotides of the nucleic acid are codon optimized for yeast or at least 5% of the nucleic acid is codon optimized for yeast in some embodiments, the transporter comprises an amino acid replacement selected from the group consisting of 91A, 209S, 213A, 256V, 262Y, 262W, 335A, 411A and any combination thereof. In some embodiments, the pathway gene is selected from a GDP-mannose 4,6-dehydratase, a GDP-L-fucose synthase, and an alpha-1,2-fucosyl transferase. In some embodiments, the microorganism comprises a second heterologous pathway gene. In some embodiments, the HMO is selected from the group consisting of 2′-fucosyllactose (2′-FL), 3′-fucosyllactose (3′-FL), 3′-sialyllactose (3′-SL), 6′-sialyllactose (6′-SL), lacto-N-neotetraose (LNnT), lacto-N-tetraose (LNT), sialyllacto-N-tetraose a (LST a), sialyllacto-N-neotetraose c (LST c), lacto-difucotetraose (LDFT) and lacto-N-fucopentaose I (LNFP I). In some embodiments, the HMO is 2′-fucosyllactose. In some embodiments, the microorganism is an Ascomycetes fungus. In some embodiments, the Ascomycetes fungus is selected from the group consisting of a Saccharomyces spp., a Schizosaccharomyces spp. and a Pichia spp. In some embodiments, the Ascomycetes fungus is selected from the group consisting of Trichoderma, Kluyveromyces, Yarrowia, Aspergillus, and Neurospora. In some embodiments, one or both of the heterologous CDT-1 transporter and the pathway gene are integrated into the yeast chromosome. In some embodiments, one or both of the heterologous CDT-1 transporter and the pathway gene are episomal. In some embodiments, the microorganism comprises a set of pathway genes for production of the HMO. In some embodiments, the set comprises GDP-mannose 4,6-dehydratase (GMD), a GDP-L-fucose synthase (GFS), and a fucosyl transferase (FT). In some embodiments, the set comprises GDP-mannose 4,6-dehydratase, a GDP-L-fucose synthase, and an alpha-1,2-fucosyl transferase and wherein the HMO is 2′-FL. In some embodiments, the set comprises a bifunctional fucokinase/L-fucose-1-P-guanylyltransferase. In some embodiments, the set comprises an enzyme capable of converting fucose and ATP to fucose-1-phosphate and an enzyme capable of converting the fucose-1-phosphate and GTP to GDP-fucose, and a glucosyl transferase. In some embodiments, the glucosyl transferase is an □-1,2-fucosyl transferase and wherein the HMO is 2′-FL. In some embodiments, the set of pathway genes comprises Gmd, WcaG and WbgL. In some embodiments, the GDP-mannose 4,6-dehydratase is selected from SEQ ID Nos. 17-19, 42, and 61-63 or a variant having at least 85% homology thereto. In some embodiments, the GDP-L-fucose synthase is selected from SEQ ID Nos. 20-23 or a variant having at least 85% homology thereto. In some embodiments, the alpha-1,2-fucosyl transferase is selected from SEQ ID Nos. 26-40 or a variant having at least 85% homology thereto.

In another aspect, a method of producing an oligosaccharide comprising culturing a microorganism described herein in a culture medium and recovering the oligosaccharide is provided herein.

In another aspect, a method of isolating an HMO comprising: providing a culture medium with at least one carbon source; providing a microorganism described herein; and culturing the microorganism in the culture medium; wherein a substantial portion of the HMO is exported into the culture medium is provided.

In another aspect, a method of isolating an HMO comprising: providing a culture medium with at least one carbon source; providing a microorganism capable of producing and exporting an HMO, wherein the microorganism comprises a heterologous transporter and one or more heterologous HMO production gene; and culturing the microorganism in the culture medium; wherein a substantial portion of the HMO is exported into the culture medium is provided.

As described above, certain embodiments are applicable to any method described herein. For example, in some embodiments, the HMO is 2-fucosyllactose, lacto-N-tetraose, lacto-N-neotetraose, 3′-sialyllactose, or 6′-sialyllactose di-fucosyllactose. In some embodiments, the method further comprising separating the culture medium from the microorganism. In some embodiments, the method further comprising isolating the HMO from the culture medium. In some embodiments, the heterologous transporter is CDT-1, CDT-2 or a variant thereof. In some embodiments, the HMO is 2′-FL. In some embodiments, the heterologous transporter gene is a CDT-1 variant comprising an amino acid sequence having one or more amino acid replacements at positions corresponding to amino acid positions 91, 209, 213, 256, 262, 335, 411 of SEQ ID NO:1. In some embodiments, the CDT-1 is encoded by a codon optimized nucleic acid. In some embodiments, the nucleic acid is optimized for yeast. In some embodiments, at least 5% of the nucleic acid is codon optimized. In some embodiments, at least 90 nucleotides of the nucleic acid are codon optimized. In some embodiments, the transporter comprises an amino acid replacement selected from the group consisting of 91A, 209S, 213A, 256V, 262Y, 262W, 335A, 411A and any combination thereof. In some embodiments, the heterologous gene is selected from a GDP-mannose 4,6-dehydratase, a GDP-L-fucose synthase, and an alpha-1,2, fucosyl transferase. In some embodiments, the export of the HMO is increased as compared to a parental yeast strain that does not contain the heterologous transporter. In some embodiments, the heterologous transporter is capable of importing lactose and exporting the HMO. In some embodiments, the culture medium comprises lactose. In some embodiments, the ratio of the HMO in the culture medium to total HMO produced by the microorganism is at least about 1:1, 1.5:1, 2:1, 2.5:1, 3:1, 3.5:1, 4:1 or greater than 4:1. In some embodiments, the HMO is selected from the group consisting of 2′-fucosyllactose (2′-FL), 3′-fucosyllactose (3′-FL), 3′-sialyllactose (3′-SL), 6′-sialyllactose (6′-SL), lacto-N-neotetraose (LNnT), lacto-N-tetraose (LNT), sialyllacto-N-tetraose a (LST a), sialyllacto-N-neotetraose c (LST c), lacto-difucotetraose (LDFT) and lacto-N-fucopentaose I (LNFP I).

In another aspect, a method of producing an HMO comprising: providing a culture medium with at least one carbon source; providing a microorganism capable of producing and exporting an HMO, wherein the microorganism expresses a heterologous transporter and one or more heterologous genes for the production of the HMO; and culturing microorganism in the culture medium; wherein a substantial portion of the HMO is exported into the culture medium is provided.

As described above, certain embodiments are applicable to any method described herein. For example, in some embodiments, the method further comprises separating the culture medium from the microorganism. In some embodiments, the method further comprises isolating the HMO from the culture medium. In some embodiments, the heterologous transporter is CDT-1, CDT-2 or a variant thereof. In some embodiments, the HMO is 2′-FL. In some embodiments, the transporter is a CDT-1 variant comprising an amino acid sequence having one or more amino acid replacements at positions corresponding to amino acid positions 91, 209, 213, 256, 262, 335, 411 of SEQ ID NO:4. In some embodiments, the CDT-1 is encoded by a codon optimized nucleic acid. In some embodiments, at least the first 90 nucleotides of the nucleic acid are codon optimized for yeast or at least 5% of the nucleic acid is codon optimized for yeast. In some embodiments, the transporter comprises an amino acid replacement selected from the group consisting of 91A, 209S, 213A, 256V, 262Y, 262W, 335A, 411A, and any combination thereof. In some embodiments, the heterologous gene is selected from a GDP-mannose 4,6-dehydratase, a GDP-L-fucose synthase, and an alpha-1,2-fucosyl transferase. In some embodiments, the export of the HMO is increased as compared to a parental microorganism that does not contain the heterologous transporter. In some embodiments, the heterologous transporter is capable of importing lactose and exporting the HMO. In some embodiments, the culture medium comprises lactose. In some embodiments, the ratio of the HMO in the culture medium to total HMO produced by the microorganism is at least about 1:1, 1.5:1, 2:1, 2.5:1, 3:1, 3.5:1, 4:1 or greater than 4:1. In some embodiments, the HMO is selected from the group consisting of 2′-fucosyllactose (2′-FL), 3′-fucosyllactose (3′-FL), 3′-sialyllactose (3′-SL), 6′-sialyllactose (6′-SL), lacto-N-neotetraose lacto-N-tetraose (LNT), sialyllacto-N-tetraose a (LST a), sialyllacto-N-neotetraose c (LST c), lacto-difucotetraose (LDFT) and lacto-N-fucopentaose I (LNFP 1), In some embodiments, the microorganism is according to any one of claims 1-29.

In another aspect, a product suitable for animal consumption comprising the HMO produced by the microorganism described herein or according to the method described herein and at least one additional ingredient acceptable for animal consumption.

In another aspect, a product suitable for animal consumption comprising the microorganism described herein and optionally at least one additional ingredient acceptable for animal consumption.

As described above, certain embodiments are applicable to any product described herein. For example, in some embodiments, the product is suitable for human consumption. In some embodiments, the product is an infant formula, an infant food, a nutritional supplement or a prebiotic product. In some embodiments, the product is suitable for mammalian consumption. In some embodiments, the product further comprising at least one additional human milk oligosaccharide. In some embodiments, the additional ingredient is selected from a protein, a lipid, a vitamin, a mineral or any combination thereof. In some embodiments, the product is suitable for use as an animal feed.

In another aspect, a product suitable for animal consumption comprising the microorganism according described herein, the HMO produced by the microorganism described herein or according to the method described herein and at least one additional consumable ingredient.

As described above, certain embodiments are applicable to any product described herein. For example, in some embodiments, the product is suitable for human consumption. In some embodiments, the product is an infant formula, an infant food, a nutritional supplement or a prebiotic product. In some embodiments, the product is suitable for mammalian consumption. In some embodiments, the product further comprises at least one additional human milk oligosaccharide. In some embodiments, the additional consumable ingredient is selected from a protein, a lipid, a vitamin, a mineral or any combination thereof. In some embodiments, the product is suitable for use as an animal feed.

### Definitions

For convenience, certain terms employed in the specification, examples, and appended claims are collected here.

As used herein, the singular forms “a,” “an” and “the” are intended to include the plural forms as well, unless the context clearly indicates otherwise.

The term “about” means within an acceptable error range for the particular value as determined by one of ordinary skill in the art, which will depend in part on how the values measured or determined, i.e., the limitations of the measurement system. Where the terms “about” or “approximately” are used in the context of compositions containing amounts of ingredients or conditions such as temperature, these values include the stated value with a variation of 0-10% around the value (X±10%).

The terms “including,” “includes,” “having,” “has,” “with,” or variants thereof are inclusive in a manner similar to the term “comprising.” The term “consisting” and the grammatical variations of consist encompass embodiments with only the listed elements and excluding any other elements. The phrases “consisting essentially of” or “consists essentially of” encompass embodiments containing the specified materials or steps and those including materials and steps that do not materially affect the basic and novel characteristic(s) of the embodiments.

Ranges are stated in shorthand to avoid having to set out at length and describe each and every value within the range. Therefore, when ranges are stated for a value, any appropriate value within the range can be selected, and these values include the upper value and the lower value of the range. For example, a range of two to thirty represents the terminal values of two and thirty, as well as the intermediate values between two to thirty, and all intermediate ranges encompassed within two to thirty, such as two to five, two to eight, two to ten, etc.

The term “genetic modification” as used herein refers to altering the genomic DNA in a microorganism. Typically, a genetic modification alters the expression and/or activity of a protein encoded by the altered gene. A genetic modification encompasses a “variant”, which is a gene or protein sequence that deviates from a reference gene or protein, as further detailed below.

The term “oligosaccharide” refers to saccharide multimers of varying length and includes but is not limited to: sucrose (1 glucose monomer and 1 fructose monomer), lactose (1 glucose monomer and 1 galactose monomer), maltose (1 glucose monomer and 1 glucose monomer), isomaltose (2 glucose monomers), isomaltulose (1 glucose monomer and 1 fructose monomer), trehalose (2 glucose monomers), trehalulose (1 glucose monomer and 1 fructose monomer) cellobiose (2 glucose monomers), cellotriose (3 glucose monomers), cellotetraose (4 glucose monomers), cellopentaose (5 glucose monomers), cellohexaose (6 glucose monomers), 2′-Fucosyllactose (2′-FL, 1 fucose monomer, 1 glucose monomer, and 1 galactose monomer), 3-Fucosyllactose (3′-FL, 1 fucose monomer, 1 glucose monomer, and 1 galactose monomer), 6′-Fucosyllactose (6′-FL, 1 fucose monomer, 1 glucose monomer, and 1 galactose monomer), 3′-Sialyllactose (3′-SL, 1 N-Acetylneuraminic acid monomer, 1 glucose monomer, and 1 galactose monomer), 6′-Sialyllacotse (6′-SL, 1 N-Acetylneuraminic acid monomer, 1 glucose monomer, and 1 galactose monomer), Di-fucosyllactose (DF-L, 2 fucose monomers, 1 glucose monomer, and 1 galactose monomer), Lacto-N-triose (LNT II, 1 N-acetylglucosamine monomer, 1 glucose monomer, and 1 galactose monomer), Lacto-N-neotetraose (LNnT, 1 N-acetylglucosamine monomer, 1 glucose monomer, and 2 galactose monomers), Lacto-N-tetraose (LNT, 1 N-acetylglucosamine monomer, 1 glucose monomer, and 2 galactose monomers), Lacto-N-fucopentaose I (LNFP I, 1 fucose monomer, 1 N-acetylglucosamine monomer, 1 glucose monomer, and 2 galactose monomers), Lacto-N-fucopentaose II (LNFP II, 1 fucose monomer, 1 N-acetylglucosamine monomer, 1 glucose monomer, and 2 galactose monomers), Lacto-N fucopentaose III (LNFP III, 1 fucose monomer, 1 N-acetylglucosamine monomer, 1 glucose monomer, and 2 galactose monomers), Lacto-N-fucopentaose IV (LNFP IV, 1 fucose monomer, 1 N-acetylglucosamine monomer, 1 glucose monomer, and 2 galactose monomers), Lacto-N-Fucopentaose V (LNFP V, 1 fucose monomer, 1 N-acetylglucosamine monomer, 1 glucose monomer, and 2 galactose monomers), Lacto-N-fucopentaose VI (LNFP VI, 1 fucose monomer, 1 N-acetylglucosamine monomer, 1 glucose monomer, and 2 galactose monomers), Lacto-N-hexaose (LNH, 2 N-acetylglucosamine monomers, 1 glucose monomer, and 3 galactose monomers), Lacto-N-neohexaose (LNnH, 2 N-acetylglucosamine monomer, 1 glucose monomer, and 3 galactose monomers), Monofucosyllacto-N-hexaose I (MFLNH I, 1 Fucose monomer, 2 N-acetylglucosamine monomer, 1 glucose monomer, and 3 galactose monomers), Monofucosyllacto-N-hexaose II (MFLNH II, 1 Fucose monomer, 2 N-acetylglucosamine monomer, 1 glucose monomer, and 3 galactose monomers), Difucosyllacto-N-hexaose I (LNDFH I, 2 N-acetylglucosamine monomers, 1 glucose monomer-2 fucose monomers and 3 galactose monomers), Difucosyllacto-N-hexaose II (LNDFH II, 2 N-acetylglucosamine monomers, 1 glucose monomer, 2 fucose monomers and 3 galactose monomers), Difucosyllacto-N-neohexaose (LNnDFH, 2 N-acetylglucosamine monomers, 1 glucose monomer, 2 fucose monomers and 3 galactose monomers), Difucosyl-para-lacto-N-Hexaose (DFpLNH, 2 N-acetylglucosamine monomers, 1 glucose monomer, 2 fucose monomers and 3 galactose monomers), Difucosyl-para-lacto-N neohexaose (DFpLNnH, 2 N-acetylglucosamine monomers, 1 glucose monomer, 2 fucose monomers and 3 galactose monomers), Trifucosyllacto-N-hexaose (TFLNH, 2 N-acetylglucosamine monomers, 1 glucose monomer, 3 fucose monomers and 3 galactose monomers), Sialyllacto-N-neotetraose c (LSTc, 1 N-acetylneuraminic acid monomer, 1. N-acetylglucosamine monomer, 1 glucose monomer, and 2 galactose monomers), Sialyllacto-N-tetraose a (LSTa, 1 N-acetylneuraminic acid monomer, 1 N-acetylglucosamine monomer, 1 glucose monomer, and 2 galactose monomers), Sialyllacto-N-tetraose b (LSTb, 1 N-acetylneuraminic acid monomer, 1 N-acetylglucosamine monomer, 1 glucose monomer, and 2 galactose monomers), Disialyllacto-N-tetraose (DSLN T, 2 N-acetylneuraminic acid monomers, 1 N-acetylglucosamine monomer, 1 glucose monomer, and 2 galactose monomers), FucosylSialyllacto-N-tetraose a (FLSTa, 1 fucose monomer, 1 N-acetylneuraminic acid monomers, 1 N-acetylglucosamine monomer, 1 glucose monomer, and 2 galactose monomers), FucosylSialyllacto-N-tetraose b (FLSTb, 1 fucose monomer, 1 N-acetylneuraminic acid monomers, 1 N-acetylglucosamine monomer, 1 glucose monomer, and 2 galactose monomers), Fucosylsialyllacto-N-hexaose (FSLNH, 1 fucose monomer, 1 N-acetylneuraminic acid monomers, 2 N-acetylglucosamine monomer, 1 glucose monomer, and 3 galactose monomers), Fucosylsialyllacto-N-neohexaose I (FSLNnH I, 1 fucose monomer, 1 N-acetylneuraminic acid monomers, 2 N-acetylglucosamine monomer, 1 glucose monomer, and 3 galactose monomers) and Fucosyldisialyllacto-N-hexaose II (FDSLNII II, 1 fucose monomer, 2 N-acetylneuraminic acid monomers, 2 N-acetylglucosamine monomer, 1 glucose monomer, and 3 galactose monomers).

The terms “human milk oligosaccharide”, “HMO”, and “human milk glycans” refer to oligosaccharides group that are be found in high concentrations in human breast milk. The dominant oligosaccharide in 80% of all women is 2′-fucosyllactose. Other HMOs include 3-fucosyllactose, 6′-fucosyllactose, 3′-sialyllactose, di-fucosyllactose, lacto-N-neotetraose, lacto-N-tetraose, lacto-N-fucopentaose I, lacto-N-fucopentaose II, lacto-N-fucopentaose III, lacto-N-fucopentaose IV, lacto-N-fucopentaose V, lacto-N-fucopentaose VI, lacto-N-hexaose, lacto-N-neohexaose, monofucosyllacto-N-hexaose I, monofucosyllacto-N-hexaose II, difucosyllacto-N-hexaose I, difucosyllacto-N-hexaose II, difucosyllacto-N-neohexaose, difucosyl-para-lacto-N-neohexaose, difucosyl-para-lacto-N-hexaose, trifucosyllacto-N-hexaose, sialyllacto-N-neotetraose a, sialyllacto-N-tetraose b, sialyllacto-N-tetraose c, disialyllacto-N-tetraose, fucosylsialyllacto-N-tetraose a, fucosylsialyllacto-N-tetraose b, fucosylsialyllacto-N-hexaose, fucosylsialyllacto-N-neohexaose I, fucosyldisialyllacto-N-hexaose II.

The term “degree of polymerization”, or DP, is the number of monomeric units in a macromolecule or polymer or oligomer molecule.

The term “microorganism” refers to prokaryote or eukaryote microorganisms capable of oligosaccharides production or utilization with or without modifications.

The term, “enhanced utilization” refers to an improvement in oligosaccharide production by a microorganism compared to a parental microorganism, specifically an increase in the oligosaccharides production rate, a decrease in the initial time before oligosaccharides production begins, an increase in the yield, defined as the ratio of product made to the starting material consumed, and/or a decrease in an overall time the microorganisms take to produce a given amount of an oligosaccharide.

The term “parental microorganism” refers to a microorganism that is manipulated to produce a genetically modified microorganism. For example, if a gene is mutated in a microorganism by one or more genetic modifications, the microorganism being modified is a parental microorganism of the microorganism carrying the one or more genetic modifications.

The term, “consumption rate” refers to an amount of oligosaccharides consumed by the microorganisms having a given cell density in a given culture volume in a given time period.

The term, “production rate” refers to an amount of desired compounds produced by the microorganisms having a given cell density in a given culture volume in a given time period.

The term “gene” includes the coding region of the gene as well as the upstream and downstream regulatory regions. The upstream regulatory region includes sequences comprising the promoter region of the gene. The downstream regulatory region includes sequences comprising the terminator region. Other sequences may be present in the upstream and downstream regulatory regions. A gene is represented herein in small caps and italicized format of the name of the gene, whereas, a protein is represented in all caps and non-italicized format of the name of the protein. For example, cdt-1 (italicized) represents a gene encoding the CDT-1 protein, whereas CDT-1 (non-italicized and all caps) represents CDT-1 protein.

The sequence identity of at least 60%, 65%, 70%, 75%, 80%, 85%, 90%, 95%, 96%, 97%, 98%, 99% or 100% to a reference sequence refers to a comparison made between two sequences, preferably using the BLAST algorithm. Algorithms for comparisons between two protein sequences that use protein structural information, such as sequence threading or 3D-1D profiles, are also known in the field.

A “variant” is a gene or protein sequence that deviates from a reference gene or protein. The terms “isoform,” “isotype,” and “analog” also refer to “variant” forms of a gene or a protein. The variant may have “conservative” changes, wherein a substituted amino acid has similar structural or chemical properties, e.g., replacement of leucine with isoleucine. A variant may have “nonconservative” changes, e.g., replacement of a glycine with a tryptophan. Analogous minor variations may also include amino acid deletions or insertions, or both. Suitable amino acid residues that may be substituted, inserted, or deleted, and which are “conservative” or “nonconservative” may be determined by those of skill in the art, including by using computer programs well known in the art.

“Exogenous nucleic acid” refers to a nucleic acid, DNA, or RNA, which has been artificially introduced into a cell. Such exogenous nucleic acid may or may not be a copy of a sequence or fragments thereof which is naturally found in the cell into which it was introduced.

“Endogenous nucleic acid” refers to a nucleic acid, gene, polynucleotide, DNA, RNA, mRNA, or cDNA molecule that is naturally present in a microorganism. An endogenous sequence is “native” to, i.e., indigenous to, the microorganism.

The term “mutation” refers to genetic modification to a gene including modifications to the open reading frame, upstream regulatory region, and/or downstream regulatory region.

A heterologous host cell for a nucleic acid sequence refers to a cell that does not naturally contain the nucleic acid sequence.

A “chimeric nucleic acid” comprises a first nucleotide sequence linked to a second nucleotide sequence, wherein the second nucleotide sequence is different from the sequence which is associated with the first nucleotide sequence in cells in which the first nucleotide sequence occurs naturally.

A constitutive promoter expresses an operably linked gene when RNA polymerase holoenzyme is available. Expression of a gene under the control of a constitutive promoter does not depend on the presence of an inducer.

An inducible promoter expresses an operably linked gene only in the presence of an inducer. An inducer activates the transcription machinery that induces the expression of a gene operably linked to an inducible promoter.

Microorganisms, Systems and Methods for Exporting Human Milk Oligosaccharides

I. Transporters

Provided herein are microorganisms, systems and methods for exporting oligosaccharides such as Human Milk Oligosaccharides (HMOs). In certain aspects, the present disclosure provides genetically engineered microorganisms capable of exporting oligosaccharides. For example, the microorganism described herein can export HMOs, such as 2′-fucosyllactose (2′-FL), such as into the growth medium where the microorganism resides.

In some embodiments, the microorganism is genetically engineered to express a transporter that is capable of exporting oligosaccharides from the microorganism. Exemplary transporters include a cellodextrin transporter, which is CDT-1, CDT-2, or homologs and variants thereof.

The transporter CDT-1 from the cellulolytic fungus Neurospora crassa (GenBank: E.A.A34565.1) belongs to the major facilitator superfamily (MFS) class of transporters capable of transporting molecules comprising hexoses and related carbohydrates. This class of transporters is defined in PFAM under family PF00083 (see the World Wide Web at pfam.xfam.org/family/PF00083).

CDT-1 is capable of importing cellodextrins including cellobiose, cellotriose and cellotetraose, as well as lactose into Saccharomyces cerevisiae. However, it has not be shown or used previous to the disclosure herein as an exporter of engineered products in a microorganism. Surprisingly, another transporter LAC12 from Kluyveromyces lactis is capable of importing lactose (like CDT-1), but as demonstrated herein, LAC12 does not function as an exporter for 2′-FL.

An example of CDT-1 is provided by the sequence of SEQ ID NO: 4, which is CDT-1 from Neurospora crassa (Uniprot entry Q7SCU1), Homologues of CDT-1 from microorganisms other than N. crassa, particularly, from fungi, can be used in the microorganisms and methods described herein. Non-limiting examples of the homologs of CDT-1 in the instant invention are represented by UniProt entries: A0A0B0E0J3, F8MZD6, G4U961, F7VQY4, Q7SCU1, A0A0J0XVF7, A0A0G2FA71, Q0CVN2, G4T6X5, A0A1Q5T2Z1, A0A0F7VA10, A0A1S9RFP6, A0A0U1LZX5, A0A0C2J3L3, U7PNA2, A0A0F2M9E7, A0A2I1D8G2, A0A2J5HR99, A0A2I2EZ95, A0A0C2IUQ7, U7PNU1, A0A1L7XY52, A0A2J6PQH9, A0A165JU51, A0A167P382, A0A1W2TJP3, A0A175VST0, A1CN94, S3DBB4, L7IWM4, G4NAG6, L7HX81, G4NAG7, A0A1Y2BF25, G0SC27, A0A0F7SHM7, A0A2P5HRQ8, A0A194VWR4, A0A194UTG8, B8M4C1, A0A2J6RYZ2, S8A1R7, R9UR53, Q4WR71, B0XPA9, A0A0J5PH40, A0A0K8LME8, A0A1Y2V0X9, A0A0F8VMB5, A1D134. A0A0S7E4Y9, A0A2T3AJM0, Q5B9G6, A0A2I1C7L5, A0A167H9D2, A0A2J6SE99, A0A0C4EGH0, A0A135LD10, A0A0A2I302, A0A0G4NZP3, K9G9B1, K9G7S2, A0A161ZL14, A0A0A2KJ45, A0A136JJM0, and A0A090D3T9.

(SEQ ID NO: 4)

| | |
|---|---|
| 1 | MSSHGSHDGA STEKHLATHD IAPTHDAIKI VPKGHGQTAT |
| | KPGAQEKEVR NAALFAAIKE |
| 61 | SNIKPWSKES IHLYFAIFVA FCCACANGYD GSLMTGIIAM |
| | DKFQNQFHTG DTGPKVSVIF |
| 121 | SLYTVGAMVG APFAAILSDR FGRKKGMFIG GIFIIVGSII |
| | VASSSKLAQF VVGRFVLGLG |
| 181 | IAIMTVAAPA YSIEIAPPHW RGRCTGFYNC GWFGGSIPAA |
| | CITYGCYFIK SNWSWRIPLI |
| 241 | LQAFTCLIVM SSVFFLPESP RFLFANGRDA EAVAFINKYH |
| | GNGDPNSKLV LLETEEMRDG |
| 301 | IRTDGVDKVW WDYRPLFMTH SGRWRMAQVL MISIFGQFSG |
| | NGLGYFNTVI FKNIGVTSTS |
| 361 | QQLAYNILNS VISAIGALTA VSMTDRMPRR AVLIIGTFMC |
| | AAALATNSGL SATLDKQTQR |
| 421 | GTQINLNQGM NEQDAKDNAY LHVDSNYAKG ALAAYFLFNV |
| | IFSFTYTPLQ GVIPTEALET |
| 481 | TIRGKGLALS GFIVNAMGFI NQFAGPIALH NIGYKYIFVF |
| | VGWDLIETVA WYFFGVESQG |
| 541 | RTLEQLEWVY DQPNPVKASL KVEKVVVQAD GHVSEAIVA |

Another example of cellodextrin transporter is CDT-2 from Neurospora crassa (UniProt entry: A0A2P5TEX1), CDT-2 is provided by the sequence of SEQ ID NO: 9.

(SEQ ID NO: 9)

| | |
|---|---|
| 1 | MGIFNKKPVA QAVDLNQIQE EAPQFERVDW KKDPGLRKLY |
| | FYAFILCIAS ATTGYDGMFF |
| 61 | NSVQNFETWI KYFGDPRGSE LGLLGALYQI GSIGSIPFVP |
| | LLTDNTGRKT PIIIGCVIMI |
| 121 | VGANLQATAK NLDTFMGGRT MLGFGNSLAQ IASPMLLTEL |
| | AHPQHRARLT TIYNCLWNVG |
| 181 | ALVVSWLAFG TNYINNDWSW RIPALLQAFP SIIQLLGIWW |
| | VPESPRFLIA KDKHDEALHI |
| 241 | LAKYHANGDP NHPTVQFEFR EIKETIRLEM ESTKNSSYLD |
| | FFKSRGNRYR LAILLSLGFF |
| 301 | SQWSGNAIIS NYSSKLYETA GVTDSTAKLG LSAGQTGLAL |
| | IVSVTMALLY DKLGRRLAFL |
| 361 | ASTGGMCGTF VIWTLTAGLY GEHRLKGADK AMIFFIWVFG |
| | IFYSLAWSGL LVGYAIEILP |
| 421 | YRLRGKGLMY MNMSVQCALT LNTYANPVAF DYFGPDHSWK |
| | LYLIYTCWIA AEFVFVFFMY |
| 481 | VETKGPTLEE LAKVIDGDEA DVAHIDIHQV EKEVEIHEHE |
| | GKSVA |

Other examples of cellodextrin transporter are Cellodextrin transporter cdt-g (UMProt entry: R9USL5), Cellodextrin transporter cdt-d (UniProt entry: R9UTV3), Cellodextrin transporter cdt-c (UniProt entry: R9UR53), Cellodextrin transporter CdtC (UniProt entry: S8A015), Putative Cellodextrin transporter CdtD (UniProt entry: AGA0U5GS76), Cellodextrin transporter CdtC (UniProt entry: S8AIR7), Cellodextrin transporter CdtD (UniProt entry: S8AVE0), and Putative Cellodextrin transporter cdt-c (UniProt entry: A0A0F7VA10).

The UniProt entries listed herein are incorporated by reference in their entireties. Additional homologs of CDT-1 are known in the art and such embodiments are within the purview of the invention. For example, the homologs of CDT-1 have at least 60%, 65%, 70%, 75%, 80%, 85%, 90%, 95%, or 99% sequence identity to SEQ ID NO: 1.

CDT-1 is a substrate-proton symporter from the MFS family. It facilitates the import of beta-1,4-linked disaccharides such as lactose or cellobiose out of the growth medium into the cell. Prior to the discoveries described herein, CDT-1 has been characterized as an importer of substrates such as cellobiose (such as used in the biofuel industry). For example, Ryan et al. (2014) have shown that variants of CDT-1, such as CDT-1 N209S and CDT-1-F262Y have an improved capability to import the oligosaccharide cellobiose. A variant with both mutations CDT-1-N209S/F262Y (or shortly: CDT-1SY) exhibited a further improved uptake of cellobiose. Mapping of the mutations on related MFS transporters revealed that the position N209 of the wildtype CDT-1 is predicted to interact with the oligosaccharide molecule inside the channel. However, neither CDT-1 nor any variants have been shown to be an exporter. To the contrary, outside of the discoveries herein, CDT-1 has been characterized as lacking activity that would provide utility as an exporter (see e.g., Hollands K. et al., Metab Eng. 2019 March; 52:232-242).

CDT-1-N209S/F262Y (or shortly: CDT-1SY):
SEQ ID NO: 1

| | |
|---|---|
| 1 | MSSHGSHDGA STEKHLATHD IAPTHDAIKI VPKGHGQTAT |
| | KPGAQEKEVR NAALFAAIKE |
| 61 | SNIKPWSKES IHLYFAIFVA FCCACANGYD GSLMTGIIAM |
| | DKFQNQFHTG DTGPKVSVIF |
| 121 | SLYTVGAMVG APFAAILSDR FGRKKGMFIG GIFIIVGSII |
| | VASSSKLAQF VVGRFVLGLG |
| 181 | IAIMTVAAPA YSIEIAPPHW RGRCTGFYSC GWFGGSIPAA |
| | CITYGCYFIK SNWSWRIPLI |
| 241 | LQAFTCLIVM SSVFFLYESP RYLFANGRDA EAVAFLVKYH |
| | GNGDPNSKLV LLETEEMRDG |
| 301 | IRTDGVDKVW WDYRPLFMTH SGRWRMAQVL MIS1FGQFSG |
| | NGLGYFNTVI FKNIGVTSTS |
| 361 | QQLAYNILNS VISAIGALTA VSMTDRMPRR AVLIIGTFMC |
| | AAALATNSGL SATLDKQTQR |
| 421 | GTQINLNQGM NEQDAKDNAY LHVDSNYAKG ALAAYFLFNV |
| | IFSFTYTPLQ GVIPTEALET |
| 481 | TIRGKGLALS GFIVNAMGFI NQFAGPIALH NIGYKYIFVF |
| | VGWDLIETVA WYFFGVESQG |
| 541 | RTLEQLEWVY DQPNPVKASL KVEKVVVQAD GHVSEAIVA |

CDT-1-N209S (or shortly: CDT-1s):
SEQ ID NO: 2

| | |
|---|---|
| 1 | MSSHGSRDGA STEKHLATHD IAPTHDAIKI VPKGHGQTAT |
| | KPGAQEKEVR NAALFAAIKE |
| 61 | SNIKPWSKES INTYFAIFVA FCCACANGYD GSLMTGIIAM |
| | DKFQNQHITG DTGPKVSVIF |
| 121 | SLYTVGAMVG APFAAILSDR FGRKKGMFIG GIFIIVGSII |
| | VASSSKLAQF VVGRFVLGLG |
| 181 | IAIMTVAAPA YSIEIAPPHW RGRCTGFYSC GWFGGSIPAA |
| | CITYGCYFIK SNWSWRIPLI |
| 241 | LQAFTCLIVM SSVFFLPESP RFLFANGRDA EAVAFLVKYH |
| | GNGDPNSKLV LLETEEMRDG |
| 301 | IRTDGVDKVW WDYRPLFMTH SGRWRMAQVL MISIFGQFSG |
| | NGLGYFNTVI FKNIGVTSTS |
| 361 | QQLAYNILNS VISAIGALTA VSMTDRMPRR AVLIIGTFMC |
| | AAALATNSGL SATLDKQTQR |
| 421 | GTQINLNQGM NEQDAKDNAY LHVDSNYAKG ALAANFLFNV |
| | IFSFTYTPLQ GVIPTEALET |
| 481 | TIRGKGLALS GFIVNAMGFI NQFAGPIALH NIGYKYIFVF |
| | VGWDLIETVA WYFFGVESQG |
| 541 | RTLEQLEWVY DQPNPVKASL KVEKVVVQAD GHVSEAIVA |

CDT-1-F262Y (or shortly: CDT-1y):
SEQ ID NO: 3

| | |
|---|---|
| 1 | MSSHGSHDGA STEKHLATHD IAPTHDAIKI VPKGHGQTAT |
| | KPGAQEKEVR NAALFAAIKE |
| 61 | SNIKPWSKES IHLYFAIFVA FCCACANGYD GSLMTGIIAM |
| | DKFQNQFHTG DTGPKVSVIF |
| 121 | SLYTVGAMVG APFAAILSDR FGRKKGMFIG GIFIIVGSII |
| | VASSSKLAQF VVGRFVLGLG |
| 181 | IAIMTVAAPA YSIEIAPPHW RGRCTGFYNC GWFGGSIPAA |
| | CITYGCYFIK SNWSWRIPLI |
| 241 | LQAFTCLIVM SSVFFLPESP RYLFANGRDA EAVAFLVKYH |
| | GNGDPNSKLV LLETEEMRDG |
| 301 | IRTDGVDKVW WDYRPLFMTH SGRWRMAQVL MISIFGQFSG |
| | NGLGYFNTVI FKNIGVTSTS |
| 361 | QQLAYNILNS VISAIGALTA VSMTDRMPRR AVLIIGTFMC |
| | AAALATNSGL SATLDKQTQR |
| 421 | GTQINLNQGM NEQDAKDNAY LHVDSNYAKG ALAAYFLFNV |
| | IFSFTYTPLQ GVIPTEALET |
| 481 | TIRGKGLALS GFIVNAMGFI NQFAGPIALH NIGYKYIFVF |
| | VGWDLIETVA WYFFGVESQG |
| 541 | RILEQLEWVY DQPNPVKASL KVEKVVVQAD GHVSEAIVA |

A lactose permease, a membrane protein, is a member of the major facilitator superfamily. Lactose permease can be classified as a symporter, which uses the proton gradient towards the cell to transport β-galactosides such as lactose in the same direction into the cell, in some embodiments, the lactose importer is LAC12. Homologues of LAC12 can be used in the microorganisms and methods described herein. Non-limiting examples of the homologs of LAC12 in the instant invention are represented by UniProt entries: Q9FLB5, B9FJH4, P07921, A0A1J6J8V9, A0A251TUB0, A0A0A9W3I8, D0E8H2, W0THP1, A0A1S9RK01, A0A151V9Y9, A0A1C1CDD3, W0TAG2, A0A151W5N5, A0A151VVE7, A0A151WBL8, A0A151V6X4, A0A151W4U2, A0A1C7LPV6, W0T7D8, W0T8B1, A0A1C1CKJ6, A0A1C1CH50, A0A1C1D058, A0A1C1C6W6, A0A1C1CIT2, A0A1C1CFR6, A0A2N6NU09, A0A1C1C6I1, A0A1C7LTH2, A0A2N6N8U0, A0A2N6NP59, A0A0F8AZD4, Q8X109, A0A1J6IEJ6, A0A034W1B8, A0A1C7LRQ8, A0A1C1CWY2, A0A1C1CTI7, A0A1C1CQ74, A0A1C7M6U6, A0A1C7LT95, AOA2N6NIJ0, A0A2C5X4W3, A0A1C7M1E6, A0A2118TQZ2, A0A2N6NWY5, A0A1T4IZL8, A0A1T4IZJ1, A0A1T4IZJ3, A0A1T4IZM1, A0A1T4IZL0, A0A1T4IZT8, A0A0A9NTY8, W8BTJ3, A0A1C7LK22, A0A0C9QF59, and A0A0A9WYQ6.

Other examples of lactose permease are encoded by LacY gene (UniProt entry: P02920, P22733, P47234, P18817, P59832), LacE (UniProt entry: P11162, P24400, P23531, Q4L869, Q5HE15, P50976, Q931 G6, Q8CNF7, Q5HM40, Q99S77, Q7A092, Q6GEN9, Q6G7C4, A0A0H3BYW2), LacS gene (UniProt entry: P23936, Q48624, Q7WTB2), LacP (UniProt entry: O33814).

The Uniprot entries listed herein are incorporated by reference in their entireties.

Lactose permease can be expressed in a microorganism and provide lactose uptake. In some aspects, lactose can then be used by the microorganism as a substrate for the production of other oligosaccharides such as HMOs. However, unlike a CDT transporter, a lactose permease, such as Lac12, when expressed in a microorganism does not act as an exporter with respect to oligosaccharides such as HMOs. For example, Lac12 does not export 2′-FL, when Lac12 is expressed in a yeast such as Saccharomyces cerevisiae.

As described herein, a cellobiose transporter acting as an importer within Neurospora crassa can act as an exporter when expressed in a microorganism such as when expressed in Saccharomyces cerevisiae strains producing an HMO. In some embodiments, the HMO exported by such transporter is a non-branched HMO comprised of a lactose core with modifications to the galactose ring. In some embodiments, the HMO is 2′-fucosyllactose (2′-FL), 3′-fucosyllactose (3′-FL), 3′-sialyllactose (3′-SL), 6′-sialyllactose (6′-SL), lacto-N-neotetraose (LNnT), lacto-N-tetraose (LNT), sialyllacto-N-tetraose a (LST a), sialyllacto-N-neotetraose c (LST c), lacto-difucotetraose (LDFT) or lacto-N-fucopentaose I (LNFP I). In some embodiments, the HMO is 2′-FL.

In some embodiments, the transporter for export of HMOs is a CDT-1, a CDT-2 or homolog thereof. In some embodiments, the transporter for export of HMOs is a variant, such as a mutant CDT-1, where one or more amino acids are altered as compared to a CDT-1 amino acid sequence. In some embodiments, a mutant CDT-1 for exporting HMOs comprises an amino acid sequence of SEQ ID NO: 1 or an amino acid sequence having 80%, 85%, 90%, 95%, 98%, 99% or greater than 99% homology with SEQ ID NO: 1. The mutant CDT-1 can have one or more amino acid changes that correspond to one or more of positions 91, 209, 213, 256, 262, 335, and 411 of SEQ ID NO:1. The mutant CDT-1 can comprise SEQ ID NO:1 having one or more amino acid substitutions selected from G91A, N209S, F213A, L256V, F262Y, F262W, F335A, S411A. In some embodiments, the mutant CDT-1 is CDT-1 N2095 F262Y (SEQ ID NO: 1), CDT-1 G91A. (SEQ ID NO: 10), CDT-1 F213A (SEQ ID NO: 11), CDT-1 L256V (SEQ ID NO: 12), CDT-1 F335A (SEQ ID NO: 13), CDT-1 S411A (SEQ ID NO: 14), or CDT-1 N209S F262W (SEQ ID NO: 15). The CDT transporter, such as a CDT-1 or mutant CDT-1 when expressed in a microorganism exports HMO such as 2′-FL. For example, cdt-1sy gene (encoding CDT-1 N209S/F262Y) was expressed within a background strain (microorganism) producing 2′-FL and 2′-FL accumulation in the growth medium during a fermentation experiment was compared to the same strain without the cdt-1-sy gene. Unexpectedly, the expression of CDT-1 N209S/F262Y significantly increases the accumulation of 2′-FL within the growth medium indicating that CDT-1SY can act as an efficient substrate exporter.

| Lactose permease mutant (CDT-1 G91A) [Neurospora crassa] | |
|---|---|
| SEQ ID NO: 10 | |
| 1 | MSSHGSHDGA STEKDLATHD IAPTHDAIKI VPKGHGQTAT KPGAQEKEVR |
| | NAALFAAIKE |
| 61 | SNIKPWSKES IHLYFAIFVA FCCACANGYD ASLMTGIIAM DKFQNQFHTG |
| | DTGPKVSVIF |
| 121 | SLYTVGAMVG APFAAILSDR FGRKKGMFIG GIFIIVGSII VASSSKLAQF |
| | VVGRFVLGLG |
| 181 | IAIMTVAAPA YSIEIAPPHW RGRCTGFYNC GWFGGSIPAA CITYGCYFIK |
| | SNWSWRIPLI |
| 241 | LQAFTCLIVM SSVFFLYESP RFLFANGRDA EAVAFLVKYH GNGDPNSKLV |
| | LLETEEMRDG |
| 301 | IRTDGVDKVW WDYRPLFMTH SGRWRMAQVL MISIFGQFSG NGLGYFNTVI |
| | FKNIGVTSTS |
| 361 | QQLAYNILNS VISAIGALTA VSMTDRMPRR AVLIIGTFMC AAALATNSGL |
| | SATLDKQTQR |
| 421 | GTQINLNQGM NEQDAKDNAY LHVDSNYAKG ALAAYFLFNV IFSFTYTPLQ |
| | GVIPTEALET |
| 481 | TIRGKGLALS GFIVNAMGFI NQFAGPIALH NIGYKYIFVF VGWDLIETVA |
| | WYFFGVESQG |
| 541 | RTLEQLEWVY DQPNPVKASL KVEKVVVQAD GHVSEAIVA |
| Lactose permease mutant (CDT-1 F213A) [Neurospora Carssa] | |
| SEQ ID NO: 11 | |
| 1 | MSSHGSHDGA STEKHLATHD IAPTHDAIKI VPKGHGQTAT KPGAQEKEVR |
| | NAALFAAIKE |
| 61 | SNIKPWSKES IHLYFAIFVA FCCACANGYD GSLMTGIIAM DKFQNQFHTG |
| | DTGPKVSVIF |
| 121 | SLYTVGAMVG APFAAILSDR FGRKKGMFIG GIFIIVGSII VASSSKLAQF |
| | VVGRFVLGLG |
| 181 | IAIMTVAAPA YSIEIAPPEW RGRCTGFNNC GWAGGSIPAA CITYGCYFIK |
| | SNWSWRIPLI |
| 241 | LQAFTCLIVM SSVFFLPESP RFLFANGRDA EAVAFLVKYH GNGDPNSKLV |
| | LLETEEMRDG |
| 301 | IRTDGVDKVW WDYRPLFMTH SGRWRMAQVL MISIFGQFSG NGLGYFNTVI |
| | FKNIGVTSTS |
| 361 | QQLAYNILNS VISAIGALTA VSMTDRMPRR AVLIIGTFMC AAALATNSGL |
| | SATLDKQTQR |
| 421 | GTQINLNQGM NEQDAKDNAY LHVDSNYAKG ALAAYFLFNV IFSFTYTPLQ |
| | GVIPTEALET |
| 481 | TIRGKGLALS GFIVNAMGFI NQFAGPIALH NIGYKYIFVF VGWDLIETVA |
| | WYFFGVESQG |
| 541 | RTLEQLEWVY DQPNPVKASL KVEKVVVQAD GHVSEAIVA |
| Lactose permease mutant (CDT-1 L256V) [Neurospora crassa] | |
| SEQ ID NO: 12 | |
| 1 | MSSHGSHDGA STEKHLATHD IAPTHDAIK1 VPKGHGQTAT KPGAQEKEVR |
| | NAALFAAIKE |
| 61 | SNIKPWSKES IHLYFAIFVA FCCACANGYD GSLMTGIIAM DKFQNQFHTG |
| | DTGPKVSVIF |
| 121 | SLYTVGAMVG APFAAILSDR FGRKKGMFIG GIFIIVGSII VASSSKLAQF |
| | VVGRFVLGLG |
| 181 | IAIMTVAAPA YSIEIAPPHW RGRCTGFYNC GWFGGSIPAA CITYGCYFIK |
| | SNWSWRIPLI |
| 241 | LQAFTCLIVM SSVFFVPESP RFLFANGRDA EAVAFLVKYH GNGDPNSKLV |
| | LLETEEMRDG |
| 301 | IRTDGVDKVW WDYRPLFMTH SGRWRMAQVL MISIFGQFSG NGLGYFNTVI |
| | FKNIGVTSTS |
| 361 | QQLAYNILNS VISAIGALTA VSMTDRMPRR AVLIIGTFMC AAALATNSGL |
| | SATLDKQTQR |
| 421 | GTQINLNQGM NEQDAKDNAY LHVDSNYAKG ALAAYFLFNV IFSFTYTPLQ |
| | GVIPTEALET |
| 481 | TIRGKGLALS GFIVNAMGFI NQFAGPIALH NIGYKYIFVF VGWDLIETVA |
| | WYFFGVESQG |
| 541 | RTLEQLEWVY DQPNPVKASL KVEKVVVQAD GHVSEAIVA |
| Lactose permease mutant (CDT-1 F335A) [Neurospora crassa] | |
| SEQ ID NO: 13 | |
| 1 | MSSHGSHDGA STEKHLATHD IAPTHDAIKI VPKGHGQTAT KPGAQEKEVR |
| | NAALFAAIKE |
| 61 | SNIKPWSKES IHLYFAIFVA FCCACANGYD GSLMTGIIAM DKFQNQFHTG |
| | DTGPKVSVIF |
| 121 | SLYTVGAMVG APFAAILSDR FGRKKGMFIG GIFIIVGSII VASSSKLAQF |
| | VVGRFVLGLG |
| 181 | IAIMTVAAPA YSIEIAPPHW RGRCTGFYNC GWFGGSIPAA CITYGGYFIK |
| | SNWSWRIPLI |
| 241 | LQAFTCLIVM SSVFFLPESP RFLFANGRDA EAVAFLVKYH GNGDPNSKLV |
| | LLETEEMRDG |
| 301 | IRTDGYDKVW WDYRPLFMTH SGRWRMAQVL MISIAGQFSG NGLGYFNTVI |
| | FKNIGVTSTS |
| 361 | QQLAYNILNS VISAIGALTA VSMTDRMPRR AVLIIGTFMC AAALATNSGL |
| | SATLDKQTQR |
| 421 | GTQINLNQGM NEQDAKDNAY LHVDSNYAKG ALAAYFLFNV IFSFTYTPLQ |
| | GVIPTEALET |
| 481 | TIRGKGLALS GFIVNAMGFI NQFAGPIALH NIGYKYIFVF VGWDLIETVA |
| | WYFFGVESQG |
| 541 | RTLEQLEWVY DQPNPVKASL KVEKVVVQAD GHVSEAIVA |
| lactose permease mutant (CDT-1 S411A) [Neurospora crassa] | |
| SEQ ID NO: 14 | |
| 1 | MSSHGSHDGA STEKHLATHD IAPTHDAIKI VPKGHGQTAT KPGAQEKEVR |
| | NAALFAAIKE |
| 61 | SNIKPWSKES IHLYFAIFVA FCCACANGYD GSLMTGIIAM DKFQNQFHTG |
| | DTGPKVSVIF |
| 121 | SLYTVGAMVG APFAAILSDR FGRKKGMFIG GIFIIVGSII VASSSKLAQF |
| | VVGRFVLGLG |
| 181 | IAIMTVAAPA YSIEIAPPHW RGRCTGFYNC GWFGGSIPAA CITYGCYFIK |
| | SNWSWRIPLI |
| 241 | LQAFTCLIVM SSVFFLPESP RFLFANGRDA EAVAFLVKYH GNGDPNSKLV |
| | LLETEEMRDG |
| 301 | IRTDGVDKVW WDYRPLFMTH SGRWRMAQVL MISIFGQFSG NGLGYFNTVI |
| | FKNIGVTSTS |
| 361 | QQLAYNILNS VISAIGALTA VSMTDRMPRR AVLIIGTFMC AAALATNSGL |
| | AATLDKQTQR |
| 421 | GTQINLNQGM NEQDAKDNAY LHVDSNYAKG ALAAYFLFNV IFSFTYTPLQ |
| | GVIPTEALET |
| 481 | TIRGKGLALS GFIVNAMGFI NQFAGPIALH NIGYKYIFVF VGWDLIETVA |
| | WYFFGVESQG |
| 541 | RTLEQLEWVY DQPNPVKASL KVEKVVVQAD GHVSENIVA |
| lactose permease mutant (CDT-1 N209S F262W) [Neurospora crassa] | |
| SEQ ID NO: 15 | |
| 1 | MSSHGSHDGA STEKHLATHD IAPTHDAIKI VPKGHGQTAT KPGAQEKEVR |
| | NAALFAAIKE |
| 61 | SNIKPWSKES IHLYFAIFYA FCCACANGYD GSLMTGIIAM DKFQNQFHTG |
| | DTGPKVSVIF |
| 121 | SLYTVGAMVG APFAAILSDR FGRKKGMFIG GIFIIVGSII VASSSKLAQF |
| | VVGRFVLGLG |
| 181 | IAIMTVAAPA YSIEIAPPHW RGRCTGFYSC GWFGGSIPAA CITYGCYFIK |
| | SNWSWRIPLI |
| 241 | LQAFTCLIVM SSVFFLPESP RWLFANGRDA EAVAFLVKYH GNGDPNSKLV |
| | LLETEEMRDG |
| 301 | IRTDGVDKVW WDYRPLFMTH SGRWRMAQVL MISIFGQFSG NGLGYFNTVI |
| | FKNIGVTSTS |
| 361 | QQLAYNILNS VISAIGALTA VSMTDRMPRR AVLIIGTFMC AAALATNSGL |
| | SATLDKQTQR |
| 421 | GTQINLNQGM NEQDAKDNAY LHVDSNYAKG ALAAYFLFNV IFSFTYTPLQ |
| | GYIPTEALET |
| 481 | TIRGKGLALS GFIVNAMGFI NQFAGPIALH NIGYKYIFVF VGWDLIETVA |
| | WYFFGVESQG |
| 541 | RTLEQLEWVY DQPNPVKASL KVEKVVVQAD GHVSEAIVA |
| lactose permease mutant (CDT-1 209S 262Y first 30 amino acid | |
| codons optimized by yeast) [Neurospora carssa] | |
| SEQ ID NO: 16 | |
| 1 | ATGTCCTCTC ATGGTTCTCA TGATGGTGCT TCTACTGAAA AACATTTGGC |
| | CACTCATGAT |
| 61 | ATTGCTCCAA CTCATGATGC TATCAAGATC GTGCCCAAGG GCCATGGCCA |
| | GACAGCCACA |
| 121 | AAGCCCGGCG CCCAAGAGAA GGAGGTCCGC AACGCCGCCC TATTTGCGGC |
| | CATCAAGGAG |
| 181 | TCCAATATCA AGCCCTGGAG CAAGGAGTCC ATCCACCTCT ATTTCGCCAT |
| | CTTCGTCGCC |
| 241 | TTTTGTTGTG CATGCGCCAA CGGTTACGAT GGTTCACTCA TGACCGGAAT |
| | CATCGCTATG |
| 301 | GACAAGTTCC AGAACCAATT CCACACTGGT GACACTGGTC CTAAAGTCTC |
| | TGTCATCTTT |
| 361 | TCTCTCTATA CCGTGGGTGC CATGGTTGGA GCTCCCTTCG CTGCTATCCT |
| | CTCTGATCGT |
| 421 | TTTGGCCGTA AGAAGGGCAT GTTCATCGGT GGTATCTTTA TCATTGTCGG |
| | CTCCATTATT |
| 481 | GTTGCTAGCT CCTCCAAGCT CGCTCAGTTT GTCGTTGGCC GCTTCGTTCT |
| | TGGCCTCGGT |
| 541 | ATCGCCATCA TGACCGTTGC TGCCCCGGCC TACTCCATCG AAATCGCCCC |
| | TCCTCACTGG |
| 601 | CGCGGCCGCT GCACTGGCTT CTACAgCTGC GGTTGGTTCG GAGGTTCGAT |
| | TCCTGCCGCC |
| 661 | TGCATCACCT ATGGCTGCTA CTTCATTAAG AGCAACTGGT CATGGCGTAT |
| | CCCCTTGATC |
| 721 | CTTCAGGCTT TCACGTGCCT TATCGTCATG TCCTCCGTCT TCTTCCTCCC |
| | AGAATCCCCT |
| 781 | CGCTaCCTAT TTGCCAACGG CCGCGACGCT GAGGCTGTIG CCTTTCTTGT |
| | CAAGTATCAC |
| 841 | GGCAACGGCG ATCCCAATTC CAAGCTGGTG TTGCTCGAGA CTGAGGAGAT |
| | GAGGGACGGT |
| 901 | ATCAGGACCG ACGGTGTCGA CAAGGTCTGG TGGGATTACC GCCCGCTCTT |
| | CATGACCCAC |
| 961 | AGCGGCCGCT GGCGCATGGC CCAGGTGCTC ATGATCTCCA TCTTTGGCCA |
| | GTTCTCCGGC |
| 1021 | AACGGTCTCG GTTACTTCAA TACCGTCATC TTCAAGAACA TTGGTGTCAC |
| | CAGCACCGCC |
| 1081 | CAACAGCTCG CCTACAACAT CCTCAACTCC GTCATCTCCG CTATCGGTGC |
| | CTTGACCGCC |
| 1141 | GTCTCCATGA CTGATCGTAT GCCCCGCCGC GCGGTGCTCA TTATCGGTAC |
| | CTTCATGTGC |
| 1201 | GCCGCTGCTC TTGCCACCAA CTCGGGTCTT TCGGCTACTC TCGACAAGCA |
| | GACTCAAAGA |
| 1261 | GGCACGCAAA TCAACCTGAA CCAGGGTATG AACGAGCAGG ATGCCAAGGA |
| | CAACGCCTAC |
| 1321 | CTCCACGTCG ACAGCAACTA CGCCAAGGGT GCCCTGGCCG CTTACTTCCT |
| | CTTCAACGTC |
| 1381 | ATCTTCTCCT TCACCTACAC TCCCCTCCAG GGTGTTATTC CCACCGAGGC |
| | TCTCGAGACC |
| 1441 | ACCATCCGTG GCAAGGGTCT TGCCCTTTCC GGCTTCATTG TCAACGCCAT |
| | GGGCTTCATC |
| 1501 | AACCAGTTCG CTGGCCCCAT CGCTCTCCAC AACATTGGCT ACAAGTACAT |
| | CTTTGTCTTT |
| 1561 | GTCGGCTGGG ATCTTATCGA GACCGTCGCT TGGTACTTCT TTGGTGTCGA |
| | ATCCCAAGGC |
| 1621 | CGTACCCTCG AGCAGCTCGA ATGGGTCTAC GACCAGCCCA ACCCCGTCAA |
| | GGCCTCCCTA |
| 1681 | AAAGTCGAAA AGGTCGTCGT CCAGGCCGAC GGCCATGTGT CCGAAGCTAT |
| | CGTTGCTTAA |

In some embodiments, a variant of CDT-1 and related transporters for use as an HMO exporter can include one or more mutations of amino acids predicted to be near the sugar substrate binding pocket (e.g., N209S in CDT-1) or near the highly-conserved PESPR motif in the sugar porter family PF00083 (e.g., F262Y in CDT-1). Exemplary mutations include amino acids in CDT-1 predicted to be in the substrate binding pocket such as G336, Q337, N341, and G471.

In some embodiments, modifications of a microorganism expressing a transporter such as CDT-1 or a CDT-1 mutant can be engineered to increase the activity of the transporter. Non-limiting examples of genetic modifications to cdt-1 that can increase the activity of CDT-1 as a substrate exporter in the microorganisms compared to CDT-1 substrate import activity in the parental microorganisms include one or more of: a) replacement of an endogenous promoter with an exogenous promoter operably linked to the endogenous cdt-1; b) expression of a cdt-1 via an extrachromosomal genetic material; c) integration of one or more copies of cdt-1 into the genome of the microorganism; (d) a modification to the endogenous cdt-1 to produce a modified CDT-1 that encodes a transporter protein that has an increased activity as a substrate exporter; e) introduction into the microorganism on extrachromosomal genetic material comprising a cdt-1 or a variant of cdt-1 (mutant cdt-1) such as encoding CDT-1 N209S F262Y or one or more of the variants described herein (e.g., CDT-1 G91A, CDT-1 F213A, CDT-1 L256V, CDT-1 F335A, CDT-1 S411A, or CDT-1 N209S F262W); f) integration into the genome of the microorganism of one or more copies of cdt-1 or a variant of cdt-1 encoding a transporter such as CDT-1 N209S F262Y, CDT-1 G91A, CDT-1 F213A, CDT-1 L256V, CDT-1 F335A, CDT-1 S411A, or CDT-1 N209S F262W; (g) introduction through extrachromosomal genetic material or through integration of a variant of cdt-1 encoding CDT-1 with one or more mutations of amino acids predicted to be near the sugar substrate binding pocket and/or the PESPR motif such as positions G336, Q337, N341, and G471; and/or (h) codon optimization of part of or all of cdt-1 or a variant of cdt-1.

Any combinations of the modifications (a) to (h) described in this paragraph are also envisioned. In some embodiments, an expression of cdt-1 or its variants is varied by utilizing different promoters or changes immediately adjacent to the introduced cdt-1 gene. For example, in certain embodiments the deletion of a URA3 cassette adjacent to an introduced cdt-1sy expression cassette leads to a further improvement of HMO export, such as 2′-FL export.

In some embodiments the endogenous promoter is replaced with an exogenous promoter that induces the expression of cdt-1 at a higher level than the endogenous promoter. In certain embodiments, the exogenous promoter is specific for the microorganism in which the exogenous promoter replaces the endogenous promoter. For example, a yeast specific exogenous promoter can be used if the microorganism being modified is a yeast. The exogenous promoter can be a constitutive promoter or inducible promoter.

Non-limiting examples of constitutive yeast specific promoters include: pCYC1, pADR1, pSTE5, pADH1, pCYC100 minimal, pCYC70 minimal, pCYC43 minimal, pCYC28 minimal, pCYC16, pPGK1, pCYC, pGPD or pTDH3. Additional examples of constitutive promoters from yeast and examples of constitutive promoters from microorganisms other than yeast are known to a skilled artisan and such embodiments are within the purview of the invention.

Non-limiting examples of inducible yeast specific promoters include: pGAL1, pMFA1, pMFA2, pSTE3, pURA3, pFIG1, pENO2, pDLD, pJEN1, pmCYC, and pSTE2. Additional examples of inducible promoters from yeast and examples of inducible promoters from microorganisms other than yeast are known to a skilled artisan and such embodiments are within the the purview of the invention.

In certain embodiments, the microorganisms comprise a modification to the wildtype cdt-1 to produce a modified cdt-1 that encodes a transporter with an increased capability to export 2′-FL from the cell.

Accordingly, in certain embodiments, modification of the wildtype cdt-1 produces a modified cdt-1 that encodes a CDT-1 with increased export rates of 2′-FL. In certain embodiments wildtype cdt-1 is mutated around the conserved PEPSR motif which is conserved in hexose transporters. In certain embodiments cdt-1 is modified leading to the production of a protein CDT-1-F262Y. The mutant CDT-1 can have one or more amino acid changes that correspond to one or more of positions 91, 209, 213, 256, 262, 262, 335, and 411 of SEQ ID NO:1. The mutant CDT-1 can comprise SEQ NO:1 having one or more amino acid substitutions selected from G91A, N209S, F213A, L256V, F262Y, F262W, F335A, S411A. In some embodiments, the mutant CDT-1 is CDT-1 N209S F262Y, CDT-1 G91A, CDT-1 F213A, CDT-1 1256V, CDT-1 F335A, CDT-1 S411A, or CDT-1 N209S F262W. The mutant CDT-1 can have one or more amino acid changes that correspond to one or more of positions predicted to be near the sugar substrate binding pocket and/or the PESPR motif such as positions G336, Q337, N341, and G471.

In certain embodiments wild-type cdt-1 is mutated around the amino acid residues within CDT-1 which are interacting with the oligosaccharide substrate. In certain embodiments cdt-1 is modified leading to the production of a protein CDT-1-N209S. In yet other embodiments cdt-1 is modified leading to the production of a protein CDT-1-N209S F262Y. In some certain embodiments cdt-1 is modified leading to the production of a protein CDT-1 G91A. In some certain embodiments cdt-1 is modified leading to the production of a protein CDT-1 F213A. In some certain embodiments cdt-1 is modified leading to the production of a protein CDT-1 L256V. In some certain embodiments cdt-1 is modified leading to the production of a protein CDT-1 F335A. In some certain embodiments cdt-1 is modified leading to the production of a protein CDT-1 S411A. In some certain embodiments cdt-1 is modified leading to the production of a protein CDT-1 N209S F262W.

In specific embodiments, a microorganism, preferably, a fungus such as a yeast, more preferably, a Saccharomyces spp., and even more preferably, S. cerevisiae is provided, the microorganism comprising the genetic modifications or the combinations of genetic modifications listed below:
1) A genetic modification producing a CDT-1 conferring the cell with oligosaccharide-, and in particular, HMO-export activity, such as 2′-FL-export activity.
2) A genetic modification producing a CDT-1 with mutated amino acid residues increasing export activity of CDT-1 for oligosaccharides, HMO-export activity, such as and in particular 2′-FL.

II. Production of HMOs in Microorganisms

HMOs are generally comprised of monosaccharides linked together, and typically with a lactose molecule at one end. Generally, the production of HMOs in microbes requires the presence of a starting monomer and one or more heterologous enzymes introduced into the microorganism. The monomer might be a monosaccharide. The monomer might be glucose, galactose, N-acetylglucosamine, fucose, and/or N-acetylneuraminic acid. For example, for the production of fucosylated HMOs, production can include i) the biosynthesis of GDP-fucose and ii) the transfer of the fucosyl domain of GDP-fucose onto an acceptor oligosaccharide. For the production of fucosylated oligosaccharide such as 2′-fucosyllactose (2′-FL) or 3-Fucosyllactose (3′-FL), the acceptor oligosaccharide is the disaccharide lactose.

GDP-fucose is synthesized from GDP-Mannose by two successive reactions: First, GDP Montrose is dehydrated by a GDP-Mannose dehydratase (GMT)) to produce GDP-4-dehydro-6-deoxy-D-mannose, Second, GDP-4-dehydro-6-deoxy-D-mannose is further reduced to GDP-L-fucose by a GDP-L-fucose synthase (GFS). In some embodiments, GDP-fucose can then be transferred to the disaccharide lactose by a fucosyl transferase (FT), forming a fucosylated oligosaccharide. In some embodiments, the FT is an alpha 1,2-fucosyl transferase. In some embodiments, the fucosylated oligosaccharide is 2″-FL or 3′-FL.

Microorganisms that exhibit increased utilization of oligosaccharides are provided. In some embodiments, the microorganism further comprises one or more heterologous HMO production gene or a construct that enhances the expression of one or more HMO production protein. As described herein “HMO production gene” expresses “HMO production protein”. As described herein, “HMO production protein” is an enzyme that participates in a pathway for HMO production. Exemplary enzymes that participate in pathways for HMO production, such as for a fucosylated HMO, are enzymes capable of converting fucose and ATP to fucose-1-phosphate, an enzyme capable of converting the fucose-1-phosphate and GTP to GDP-fucose, and/or a glucosyl transferase. Examples of HMO production protein are a GDP-Mannose dehydratase (GMD), a GDP-L-fucose synthase (GFS), and a fucosyl transferase (FT).

In certain embodiments, the microorganisms comprise one or more genetic modifications that: i) increase the activity of a GDP-Mannose dehydratase (GMD), and/or ii) increase the activity of a GDP-L-fucose synthase (GFS), and/or iii) increase the activity of glycosyl transferase such as fucosyl transferase (FT), e.g., alpha 1,2-fucosyl transferase. In certain embodiments, these genetic modifications that result in i), ii), and iii) are produced by introduction of a GDP-Mannose dehydratase gene (GMD), GDP-L-fucose synthase gene (GES), and a glycosyl transferase such as fucosyl transferase (FT), e.g., alpha 1,2-fucosyl transferase gene, respectively. In some embodiments, the microorganism comprises a heterologous GDP-Mannose dehydratase gene or a construct that enhances expression of the GDP-Mannose dehydratase. In some embodiments, the microorganism comprises a heterologous GDP-L-fucose synthase gene or a construct that enhances expression of the GDP-L-fucose synthase. In some embodiments, the microorganism comprises a heterologous glycosyl transferase such as fucosyl transferase (FT), e.g., alpha 1,2-fucosyl transferase gene or a construct that enhances expression of the glycosyl transferase such as fucosyl transferase (Fr), e.g., alpha 1,2-fucosyl transferase.

In certain embodiments, the present disclosure provides microorganisms comprising one or more genetic modifications selected from:
i) a genetic modification that introduces a GDP-Mannose dehydratase gene (GMD), or its analogues,
ii) a genetic modification that introduces a GDP-L-fucose synthase gene (GFS), or its analogues, and
iii) a genetic modification that introduces a glycosyl transferase such as fucosyl transferase (FT), e.g., alpha 1,2-fucosyl transferase gene, or its analogues.

HMOs, such as 2′-FL can be produced in a microorganism. In some embodiments, a microorganism is genetically engineered by incorporating one or more nucleic acids that encode for an enzyme for one or more steps in the production of an HMO. In some embodiments, an HMO pathway is supplied entirely by such genetic engineering. In some embodiments, an HMO pathway is comprised of one or more endogenous activities from the host microorganism, and others through genetic engineering. In yet other embodiments, the host microorganism synthesizes an HMO using endogenous activities.

In some embodiments, the HMO is 2′-fucosyllactose (2′-FL), 3′-fucosyllactose (3′-FL), 3′-sialyllactose 6′-sialyllactose (6′-SL), lacto-N-neotetraose (LNnT), lacto-N-tetraose (LNT), sialyllacto-N-tetraose a (LST a), sialyllacto-N-neotetraose c (LST c), lacto-difucotetraose (LDFT) or lacto-N-fucopentaose I (LNFP I).

In some cases, the HMO is a fucosyllactose, such as 2′-FL. In some embodiments, fucosyllactose, such as 2′-FL is synthesized in a host microorganism through a de novo pathway. For example, the pathway can comprise GMD (GDP-mannose dehydratase), GFS (GDP-fucose synthase), and FT (fucosyltransferase), where GMD supplies an enzymatic activity to convert GDP-Mannose to GDP-4-keto-6-deoxymanose. A GFS, for example, WcaG, converts GDP-4-keto-6-deoxymanose to GDP-fucose and FT converts GDP-fucose to 2′-FL. In some embodiments, the FT is an alpha 1,2-fucosyl transferase.

An example of GDP-Mannose dehydratase (GMD) is provided by the sequence of SEQ ID NOs: 17-19, which are GDP-Mannose dehydratases from Fistularia solaris, Cladosiphon okamuranus, and Cladosiphon okamuranus, respectively. Homologues of GMD from microorganisms other than Fistularia solaris and Cladosiphon okamuranus, in particular, from other heterokontophytes and from fungi, can be used in the microorganisms and methods described herein. Non-limiting examples of the homologs of GMD in the instant invention are represented by UniProt entries: P93031, O60547, Q18801, Q51366, Q93VR3, P0AC88, Q9VMW9, O45583, A3C4S4, Q9SNY3, Q8K0C9, Q8K3X3, Q9JRN5, Q56872, A0A1B4XBH2, P55354, O85713, Q06952, Q1ZXF7, Q56598, P0AC90, P0AC91, P0AC89, B9UJ29, A8Y0L5, O67175, P71790, A0A1H3VGZ0, A0A078KV89, Q7UVN9, Q7NMK1, Q89TZ1, A0A132P8J4, P72586, Q2R1V8, A0A0G1U600, A2Z7B3, D4ZMX8, K9QEY2, L0A7V1, C3SCZ0, B5W8Q3, K1XEL2, A0A0G1FQB5, H1WLZ0, and Q63JM19.

The UniProt entries listed herein are incorporated by reference in their entireties. Additional homologs of GMD are known in the art and such embodiments are within the purview of the invention. For example, the homologs of GMD have at least 60%, 65%, 70%, 75%, 80%, 85%, 90%, 95%, or 99% sequence identity to SEQ ID NOs: 17-19 and 42.

GDP-mannose 4,6-dehydratase (GMD; EC 4.2.1.47) catalyzes the conversion of GDP-mannose to GDP-4-keto-6-deoxymannose, the first step in the synthesis of GDP-fucose from GDP-mannose, using NAD+ as a cofactor. This enzyme belongs to the family of lyases, specifically the hydro-lyases, which cleave carbon-oxygen bonds. The systematic name of this enzyme class is GDP-mannose 4,6-hydro-lyase (GDP-4-dehydro-6-deoxy-D-mannose-forming). Other names in common use include guanosine 5′-diphosphate-D-mannose oxidoreductase, guanosine diphosphomannose oxidoreductase, guanosine diphosphomannose 4,6-dehydratase, GDP-D-mannose dehydratase, GDP-D-mannose 4,6-dehydratase, Gmd, and GDP-mannose 4,6-hydro-lyase. This enzyme participates in fructose and mannose metabolism. It employs one cofactor, NAD+.

In some embodiments, GMD and/or GFS are derived from E. coli, Helicobacter pylori, Arabidopsis thaliana, and/or Mortierella alpina (Ren et al., Biochem Biophys Res Commun. 2010 Jan. 22; 391(4):1663-9; Hollands K. et al., Metab Eng. 2019 March; 52:232-242), in some embodiments, GMD is encoded by one of the sequences listed in Table 1 or a variant thereof.

Many of the proteins involved in GDP-fucose synthesis presented here have been identified in heterokontophytes, a group of algae which includes diatoms and kelps and which had been shown to contain large amounts of fucose in their cell walls. In addition, fusion proteins which appear to consist of a GMD and a GFS protein domain were identified.

TABLE 1
GMD activity:

| SEQ ID NO | Organism | Organism description |
|---|---|---|
| SEQ ID NO 17 | Fistularia solaris | Diatom |
| SEQ ID NO 18 | Cladosiphon okamuranus | Kelp |
| SEQ ID NO 19 | Cladosiphon okamuranus | Kelp |
| SEQ ID NO: 42 | Escherichia coli | N/A |

| SEQ ID NO: 17 | |
|---|---|
| 1 | MSSERKCALI TGITGQDGSY LTELLLFKGY EVHGIVRRSS CFNTGRIDHL |
| | YKDRHETGVK |
| 61 | LFLHYGDLCD ATNLISIISN VKPTEVYNLG AMSHVKVSED MPEYTADCDG |
| | VGVLRMLDAI |
| 121 | RAAGMEKTVK FYQASTSELY GKVQEVPQSE TTPFYPRSPY AVAKQYAFWI |
| | LVNYREAYGM |
| 181 | HLTNGILFNH ESPRRGRTFV TRKITCGVAA IHHGKQKTLF LGNLDAKRDW |
| | GHARDYVEGM |
| 241 | WRMLQQETSD DYVLATGETH TVREFVEKAF AVVNTTVQWQ GEKGTVDEVG |
| | VDAADPSRIL |
| 301 | VRIDPRYFRP TEVDLLLGNP AKAKEKLGWS SSTPFDALVK EMVEADLAIL |
| | RGEMADADNT |
| 361 | FD |
| SEQ ID NO: 18 | |
| 1 | MAEPETKKTK VDEGTVKKAI ITGITGQDGS YLAEFLLEKG YEVHGIIRRS |
| | SSFNTQRIDH |
| 61 | IYRDRHESAV RLKLHYGDLT DSTNLMHIIY EVQPDEIYNL GAMSHVKVSF |
| | EMSEYTAEAD |
| 121 | GVGVLRLLNA IRSAGLEKKT RLYQASTSEL YGKVQEIPQK ETTPFYPRSP |
| | YGVAKQFGYW |
| 181 | MLINYREAYG MHLTNGILFN HESPRRGPTF VTRKITRAVA RIHRGKQKCI |
| | YLGNLDAKRD |
| 241 | WGHAKDYIKG MWLMVQRDEP SDYVLSTGEC HSVKEFVQEA FAYVGIDITW |
| | VGEGVEEYGH |
| 301 | VKGDPENVLV RVDPRYFRPT EVELLLGDCT KAKKELGWVP EITFKELVKD |
| | MMQADIANVD |
| 361 | AGNDHT |
| SEQ ID NO: 19 | |
| 1 | MQKTALITGI TGQDGAYLAE LLLEKGYTVH GIKRRSSSFN TGRIDHLYQD |
| | PHDRDVKLHL |
| 61 | HYGDMTDSTN LIRIMQETQP DEVYNLAAQS HVQVSFETPE YTGNADALGT |
| | LRLLEAIRLL |
| 121 | GLSEKTRFYQ ASTSELYGKV QEVPQSETTP FYPRSPYAAA KLYAYWIVVN |
| | YREAYGMHAS |
| 181 | NGILFNHESP IRGETFVTRK ITRAAAAIKL GLQDKLYLGN LDAERDWGHA |
| | KDYVRGMWLM |
| 241 | LQQDKADDYV LATGEKHSVR EFVEQAFAEL EINIRWEGRG LDEQGFDTKT |
| | EKAVVAVDPR |
| 301 | YFRPTEVDLL LGSPKKARKA LGWAPTTPFR DMIKQMVRSD LNSVSEDSKK |
| | QGSASWIKTG |
| SEQ ID NO: 42 | |
| 1 | MSKVALITGV TGQDGSYLAE FLLEKGYEVH GIKRRASSFN TERVDIHYQD |
| | PHTCNPKFHL |
| 61 | HYGDLSDTSN LTRILREVQP DEVYNLGAMS HVAVSFESPE YTADVDAMGT |
| | LRLLEAIRFL |
| 121 | GLEKKTRFYQ ASTSELYGLV QEIPQKETTP FYPRSPYAVA KLYAYWITVN |
| | YRESYGMYAC |
| 181 | NGILFNHESP RRGETFVTRK ITRAIANIAQ GLESCLYLGN MDSLRDWGHA |
| | KDYVKMQWMM |
| 241 | LQQEQPEDFV IATGVQYSYR QFVEMAAAQL GIKLRFEGTG VEEKGVVVSV |
| | TGHDAPGVKP |
| 301 | GDVIIAVDPR YFRPAEVETL LGDPTKAHEK LGWKPEITLR EMVSEMVAND |
| | LEAAKKHSLL |
| 361 | KSHGYDVAIA LES |
| GMD from Helicobacter pylori | |
| SEQ ID NO: 60 | |
| 1 | MKEKIALITG VTGQDGSYLA EYLLNLGYEV HGLKRRSSSI NTSRIDHLYE |
| | DLHSDHKRRF |
| 61 | FLHYGDMTDS SNLIHLIATT KPTEIYNLAA QSHVKVSFET PEYTANADGI |
| | GTLRILEAMR |
| 121 | ILGLEKKTRF YQASTSELYG EVLETPQNEN TPFNPRSVYA VAKMYAFYIT |
| | KNYREAYNLF |
| 181 | AVNGILFNHF SRVRGETFVT RKITRAASAI AYNLTDCLYL GNLDAKRDWG |
| | HAKDYVKMMH |
| 241 | LMLQAPIPQD YVIATGKTTS VRDFVKMSFE FIGINLEFQN TGIKEIGLIK |
| | SVDEKRANAL |
| 301 | KLNISFLKKG QIVVRIDERY FRPTEVDLLL GDPTKAEKEL DWVREYDLKE |
| | LVKDMLEYDL |
| 361 | KECQKNLYLQ DGGYILRNTY E |
| GMD from Arabidopsis thaliana: | |
| SEQ ID NO: 61 | |
| 1 | MASENNGSRS DSESITAPKA DSTVVEPRKI ALITGITGQD GSYLTEFLLG |
| | KGYEVHGLIR |
| 61 | RSSNFNTQRI NHIYIDPHNV NKALMKLHYA DLTDASSLRR WIDVIKPDEV |
| | YNLAAQSHVA |
| 121 | VSFEIPDYTA DVVATGALRL LEAVRSHTID SGRTVKYYQA GSSEMFGSTP |
| | PPQSETTPFH |
| 181 | PRSPYAASKC AAHWYTVNYR EAYGLFACNG ILFNHFSPRR GENFVTRKIT |
| | RALGRIKVGL |
| 241 | QTKLFLGNLQ ASRDWGFAGD YVEAMWLMLQ QEKPDDYVVA TEEGHTVEEF |
| | LDVSFGYLGL |
| 301 | NWKDYVEIDQ RYFRPAEVDN LQGDASKAKE VLGWKPQVGF EKLVKMMVDE |
| | DLELAKREKV |
| 361 | LVDAGYMDAK QQP |
| GMD from Mortierella alpine | |
| SEQ ID NO: 62 | |
| 1 | MSSPIEVVNM SPADYRNRKV ALITGITGQD GSYLAELLIE KGYQVHGIIR |
| | RSSSFNTGRI |
| 61 | EHLYKDAHEN PKMRLHHGDL TDSTCLVHII SKVLPTEIYN LGAQSHVKVS |
| | FDMSEYTADV |
| 121 | DAVGTLRLLD AIRTCGLSHL VRFYQASTSE LYGKVAEIPQ SETTPFYPRS |
| | PYGVAKMYAY |
| 181 | WITINYREAY DMYACNGILF NHESPRRGRT FVTRKITCAV ASIHLGKQEC |
| | LYLGNLDAKR |
| 241 | DWGHARDYVE GMWRMLQQET AEDFVLATGE MHTVREFVEK SFKAIGSTIR |
| | WEGSAEEEVG |
| 301 | LDEKGVIRVR VDPAYYRPTE VELLLGNPAK ANEKLGWKRQ VEFDALVEEM |
| | VKSDLIGVAA |
| 361 | GDVFN |

An example of a GFS (GDP-fucose synthase) is provided by the sequence of SEQ ID NOs: 20-23, which are GDP-L-fucose synthases from Cladosiphon okamuranus, Phaeodactylum tricornutum, Saccharina japonica, and Mucor circinelloides circinelloides 1006PhL, respectively. Homologues of GFSs from microorganisms other than Cladosiphon okamuranus, Phaeodactylum tricornutum, Saccharina japonica, and Mucor circinelloides f. circinelloides 1006PhL, in particularly from heterokontophytes and from fungi, can be used in the microorganisms and methods described herein. Non-limiting examples of the homologs of GFSs in the instant invention are represented by UniProt entries: Q13630, P32055, O49213, P23591, Q9W1X8, Q9LMU0, G5EER4, Q8K3X2, P33217, Q5RBE5, F0F7M8, Q67WR2, P55353, Q67WR5, D9RW33, F2KZP1, G1WDT9, D7NG24, C9MLN8, Q9S5F8, X6PWX2, H1HNE5, D1QPT8, G6AG96, I0TA81, G1VAH6, A0A0K1NMZ0, U2KFA0, F0H551, A0A2K9HDD8, A0A095YQN3, D3I452, A0A096ARU1, A0A095ZVW3, A0A096ACH9, A0A1B1IBP6, Q55C77, A0A1F0MVW9, A0A1F0P341, A0A1T4MGU5, W4UTD5, A0A0G0Z978, QSV3C6, A0A2U0U1K6, A0A2T4T802, and A0A2T4TH79.

The UMProt entries listed herein are incorporated by reference in their entireties. Additional homologs of GFS's are known in the art and such embodiments are within the purview of the invention. For example, the homologs of GFS's have at least 60%, 65%, 70%, 75%, 80%, 85%, 90%, 95%, or 99% sequence identity to SEQ ID NOs: 20-23.

A GDP-L-fucose synthase (EC 1.1.1.271) is an enzyme that catalyzes the chemical reaction GDP-4-dehydro-6-deoxy-D-mannose+NADPH+H⁺<->GDP-L-fucose+NADP⁺. Thus, the three substrates of this enzyme are GDP-4-dehydro-6-deoxy-D-mannose, NADPH, and H⁺, whereas its two products are GDP-L-fucose and NADP⁺. This enzyme belongs to the family of oxidoreductases, specifically those acting on the CH—OH group of a donor with NAD⁺ or NADP⁺ as acceptor. The systematic name of this enzyme class is GDP-L-fucose:NADP⁺ 4-oxidoreductase (3,5-epimerizing). This enzyme is also called GDP-4-keto-6-deoxy-D-mannose-3,5-epimerase-4-reductase. This enzyme participates in fructose and mannose metabolism.

In some embodiments, GFS is encoded by one of the sequences listed in Table 2 or a variant thereof.

TABLE 2
GFS activity:

| SEQ ID NO | Organism | Organism description |
|---|---|---|
| SEQ ID NO 20 | Cladosiphon okamuranus | Kelp |
| SEQ ID NO 21 | Phaeodactylum tricornutum | Diatom |
| SEQ ID NO 22 | Saccharina japonica | Kelp |
| SEQ ID NO 23 | Mucor circinelloides | Soil fungus |
| | f. circinelloides 1006PhL | |

| SEQ ID NO: 20 | |
|---|---|
| 1 | MEATKAIFEK YKPTHVIHLA ARVGGLFSNL KYKVEFFREN ILINDNVMEC |
| | CRIYKVAKLV |
| 61 | SCLSTCIFPD KTTYPIDETM VHNGPPHTSN EGYAYAKRMI DVLNRCYKDE |
| | YGCNFTSVIP |
| 121 | TNIYGKGDNF SIDNGHVLPG LIHKCYKAKQ AGEDLHVWGT GSPLRQFIYN |
| | VDLGALMVWT |
| 181 | MRNYHEVDPI ILSVGEEDEV SIADAARMIA SAMDFEGNVV FDTDKSDGQF |
| | KKTACNDLLK |
| 241 | KKNPDFKFTS MQDGLKAACD WFCENTETAR K |
| SEQ ID NO: 21 | |
| 1 | MVTGGSGLVG AAIREYVEGT GALENESWIY LNSKEGDLRN RADTEKIFAK |
| | YQPTHVIHLA |
| 61 | AKVGGLFANM AQKVEFFREN ILINDNIMEC SRIYKVEKLV SFLSTCIFPD |
| | KTTYPIDETM |
| 121 | LHDGPPHPSN EGYAYAKRL1 DTMNRAYAEE YGCNFISIIP TNIYGPHDNF |
| | SIQNGHVIPG |
| 181 | LIHKCYLAKK DNTPFIIWGS GTPLRQFVYS RDLAELTVWS MREYHDPTPI |
| | TLSVDEEEEV |
| 241 | SIKDVALAVA KAMQFDGQPV FDTSKADGQF KKTACNKKLR SLKADYEFTS |
| | MPDGIQQSVD |
| 301 | WFVANYDSCR K |
| SEQ ID NO: 22 | |
| 1 | MAETSGTDAA PKKVVMVTGG TGLVGCGIKE FVESDAEAKE KEEYIFLSSK |
| | DGDIRNMEET |
| 61 | KLIFEKYKPT HVIHLAARVG GLFSNLKYKV EFFRENILIN DNVMECCRIY |
| | KVEKLVSCLS |
| 121 | TCIFPDKTTY PIDETMVHNG PPHVSNEGYA YAKRMIDVLN RCYKEEYGCN |
| | FTSVIPTNIY |
| 181 | GKGDNFSIDN GHVLPGLIHK CYKAKQAGED LHVWGTGSPL RQFIYNVDLG |
| | ALMIWTMRNY |
| 241 | HEVDPIILSV GEEDEVSIAD AAKMIASAMD FEGNVVFDTD KSDGQFKKTA |
| | CNDLLKQKNP |
| 301 | DEKFTPMKEG LKQACEWFCE NYETARK |
| SEQ ID NO: 23 | |
| 1 | MATESVILVT GGSGLVGEAV KWVIENDKSE RYGKKENEKW VFLSSKDGDL |
| | RKEQDVKAIF |
| 61 | EKYKPTRVIH LAAMVGGLEK NMKYKLDFLR ENMLMNDNIL WQSKEYNVKK |
| | VVSCLSTCIF |
| 121 | PDKTTYPIDE TMVHNGPPHE SNFGYAHGKR MIDVYNHAYH EQFGCHFTSV |
| | IPTNIFGPHD |
| 181 | NYDLEGSHVL PGLTHKCYLA KKNNTPFVVW GSGKPLRQFI YSRDLAKLFI |
| | WTLREYEEID |
| 241 | PIILSVGEKD EVSIKDVADS IVKAMDFQGE YSFDSTKADG QYKKTASNEK |
| | LMKYIPDFEF |
| 301 | TPFDVAIKES VEWFVENYDT LRK |
| GDP-L-fucose synthetase (WeaG) [Escherichia coli] | |
| SEQ ID NO: 43 | |
| 1 | MSKQRVFIAG HRGMVGSAIR RQLEQRGDVE LVLRTRDELN LLDSRAVHDF |
| | FASERIDQVY |
| 61 | LAAAKVGGIV ANNTYPADFI YQNMMIESNI IHAAHQNDVN KLLFLGSSCI |
| | YPKLAKQPMA |
| 121 | ESELLQGTLE PTNEPYAIAK IAGIKLCESY NRQYGRDYRS VMPTNLYGPH |
| | DNFHPSNSHV |
| 181 | IPALLRRFHE ATAQNAPDVV VWGSGTPMRE FLHVDDMAAA SIHVMELAHE |
| | VWLENTQPML |
| 241 | SHINVGTGVD CTIRELAQTI AKVVGYKGRV VFDASKPDGT PRKLLDVTRL |
| | HQLGWYHEIS |
| 301 | LEAGLASTYQ WFLENQDRFR G |
| GMER (WeaG) from Arabidopsis thaliana | |
| SEQ ID NO: 44 | |
| 1 | MAETIGSEVS SMSDKSAKIF VAGHRGLVGS AIVRKLQEQG FTNLVLKTHA |
| | ELDLTRQADV |
| 61 | ESFFSQEKPV YVILAAAKVG GIHANNTYPA DFIGVNLQIQ TNVIHSAYEH |
| | GVKKLLFLGS |
| 121 | SCIYPKFAPQ PIPESALLTA SLEPTNEWYA IAKIAGIKTC QAYRIQHGWD |
| | AISGMPTNLY |
| 181 | GPNDNFHPEN SHVLPALMRR FHEAKVNGAE EVVVWGTGSP LREFLHVDDL |
| | ADACVFLLDR |
| 241 | YSGLERVNIG SGQEVTIREL AELVKEVVGF EGKLGWDCTK PDGTPRKLMD |
| | SSKLASLGWT |
| 301 | PKVSLRDGLS QTYDWYLKNV CNR |
| GMER (WcaG) from Helicebacter pylori | |
| SEQ ID NO: 45 | |
| 1 | MNEIILITGA YGMVGQNTAL YFKKNKPDVT LLTPKKSELC LLDKDNVQAY |
| | LKEYKPTGII |
| 61 | HCAGRVGGIV ANMNDLSTYM VENLLMGLYL FSSALDSGVK KAINLASSCA |
| | YPKFAPNPLK |
| 121 | ESDLLNGSLE PTNEGYALAK LSVMKYCEYV SAEKGVEYKT LVPCNLYGEF |
| | DKFEEKIAHM |
| 181 | IPGLIARMHT AKLKNEKFFA MWGDGTARRE YLNAKDLARE ISLAYENIAS |
| | IPSVMNVGSG |
| 241 | VDYSIEEYYE KVAQVLDYKG VFVKDLSKPV GMQQKLMDIS KQRALKWELE |
| | IPLEQGIKEA |
| 301 | YEYYLKLLEV |
| GMFR from Mortierella alpine | |
| SEQ ID NO: 46 | |
| 1 | MSPSKSVIMV TGGSGLVGKA IDWVVENDSK YGKREGEEWV FLTSKDGNLI |
| | DPAQTKAIFE |
| 61 | KYRPTHVIHL AAKVGGLFGN MAANLDYFRD NLLINDNVLH NAKEFGVKKV |
| | VSCLSTCIFP |
| 121 | DKTSYPIDET MVHQGPPHDS NYGYSHSKRM VDVMNRAYNQ QYGCNFTSVI |
| | PTNVFGPHDN |
| 181 | FHLVNSHVIP GLIHKCYLAQ QNNTPFIMAG TGRPLRQFIY SRDLARLFIW |
| | TLREYQEITP |
| 241 | LILSVPEEEE VSIKQVGDSI VKAMGYTGDY RFDTTKADGQ YKKTASNKKL |
| | MSLNPDFQFT |
| 301 | PFDVALSETV EWFKENVDTI RK |

In some embodiments, GMD and GFS activities are supplied by a single enzyme, such as one of those listed in Table 3 or a variant thereof.

TABLE 3
genes for GFS and GMD activity

| SEQ ID NO | Organism | Organism description |
|---|---|---|
| SEQ ID NO 24 | Puniceicoccaceae | Bacterium |
| | bacterium TMED149 | |
| SEQ ID NO 25 | Cladosiphon okamuranus | Kelp |

| SEQ ID NO: 24 | |
|---|---|
| 1 | MKKALITGIT GQDGSYLAEL LLEKGYEVHG IIRRASTFNT RDHYEDPHIN |
| | GKKFLHYGDL |
| 61 | ADGVQMVKLL YNLQPDEIYH LGAQSHVRVS FDVPEYTGDV TGLGTLRLEA |
| | IREVGLNNKC |
| 121 | RFYQASSSEM FGMVQEVPQT EKTPYPRSPG CAKVYAYWLT VNYRESYNLH |
| | ATNGILFNHE |
| 181 | SPRRGETFVT RKITRAATRI KMGLQDKLYL GNLDAKRDWG YAKEYVEAMW |
| | LMLQQDSGDD |
| 241 | YVMATNETHS VKEVQETFAQ LDMDWEAFVE YDKRYERPTE VDLIGDPSKA |
| | KKQLDWEPKV |
| 301 | RFKDLVKINW EADLEIARKE AAFKAATEQS FRLMNKDAKI YVAGHRGMVG |
| | SAVVRALEEN |
| 361 | GFQSIITRTH AELDLTDQSE VRAFFQSNNI QYAVIAAAKV GGIHANNSYP |
| | AEFIYENLAI |
| 421 | AQNTIHEAYA SGVRILFLGS TCYPKFAKQP IQEASLLTDA LEPTNEAYAI |
| | AKIAGLKLCQ |
| 481 | FYRQYGVLYH SAMPTNLYGR GDNYHPENSH VMPALIRRIH EAKEVGAPEV |
| | VVWGTGKPLR |
| 541 | EFLHSEDAAS GIVHLLNIEN PPDWVNLGSG REISIGDLAQ MISSIIGYDG |
| | VLKFDTSKPD |
| 601 | GTPRKVTDIQ LISDTGWSPQ ISLEEGVASA YQEFLFELKQ GTVRF |
| SEQ ID NO: 25 | |
| 1 | MQAEFLLEKG YEVHGVKRRA SLFNTQRVDH LYEDPHDSDT RLKLHYGDLT |
| | DTSNLTRLLR |
| 61 | DIEPDEVYNL GAQSHVAVSF EAPEYTADVD ATGTLRLLEA IRELGLEEKT |
| | RFYQASTSEL |
| 121 | YGKVQEIPQS ETTPFHPRSP YAVAKMYAYW ITVNYRESYG MYACNGILFN |
| | HESPRRGETF |
| 181 | VTRKITRGLS NIAMGLEPCL YMGNIDALRD WGHAKDYVRM QWMMLQQDEP |
| | EDFVIATGVQ |
| 241 | YSVREFIRWT ARELGMELEF SGTGTDEIAR VASITGDRVK ALKVGDVVMR |
| | IDPRYFRPAE |
| 301 | VETLLGNPAK AKAKLGWVPE ITAQEMCKRI WVAGHRGMVG GAVVRRLERE |
| | DCEVICAARD |
| 361 | VVDLTRQQEV QDWMAETRPD AIIMAAAKVG GILANDTRPV DFLLQNLQIE |
| | TNIVEAAHQV |
| 421 | DVERFLFLGS SCIYPKMAPQ PIPEDSLLTG PLEPTNEWYA IAKIAGIKLM |
| | QAYRKQYGRD |
| 481 | WISAMPTNLY GPGDNYDLAS SHVLPALLRK FHEAKVAGAK HVELWGSGTP |
| | LREFMHCDDL |
| 541 | ADALVFLLQR YSGHDHVNVG SGSEVSIREL AETIAQVVGY EAEIVFDSSK |
| | PDGTPRKLMD |
| 601 | SARLHDMGWN NARSLLDGLR DTYARGTVVF KSVADEIRTV DVADYSILPV |
| | GVVQWLETDG |
| 661 | AGDSYNLASR LDFAPNPDIA VISALRPLSN LTPIQRVFHL GGGNQHILLM |
| | RMISSQPEDV |
| 721 | HNIPHLGWYM RTGVRVIVIS AALSSGGLFA IGWILQSSGH AYGRVLIGGA |
| | VFLFPMILAE |
| 781 | AVMNLARARG SFFMALLPRD IIWRTLVIAI ALGLLLALPT GWSGLQLMLI |
| | CAGSLMICLL |
| 841 | VQIRLAWGLY AGHIPPQTAP DWPNWRAQSL WLWISSLAGN ISGNLAVLII |
| | SMTLSLEAAG |
| 901 | VFFAALRLSM VLALPLNALN IAVAPRFSHL HARQDYNALQ TYGLRMTQVI |
| | ALPTLAALAL |
| 961 | IVAYGDQALS WEDSEITGGW GALCLLAIGY TLRTCAGASG VMMLMTGHER |
| | KAVRIFFQTE |
| 1021 | GLSLLVLPLA AHFYGIEGAA ACLALGVAAS SVLSNLHLRR SFRVDPGLHS |
| | VLLAPRSDQG |
| 1081 | IL |

Examples of fucosyl transferases (FTs), e.g., alpha-1,2-fucosyl transferase are provided by the sequences of SEQ ID NOs: 26-40, which are alpha 1,2-fucosyl transferases from Dictyostelium discoideum AX4, Homo sapiens, Pisum salivca, Rhizobium marinum, Herbaspirillum rubrisubalbicans, Citrobacter freundii, Lactobacillus helveticus, Neocallimastix californiae, Gracilariopsis chorda, Lactobacillus gasseri, Octopus bimaculoides, and Chryseobacterium scophthalmum, respectively. Homologues of FTs from microorganisms other than Diciyostelium discoicleum AX4, Homo sapiens, Pisum saliva, Rhizobium marinum, Herbaspirllium rubrisubalbicans, Citrobacter freundii, Lactobacillus helveticus, Neocallimastix californiae, Gracilarlopsis chorda, Lactobacillus gasseri, Octopus bimaculoides, and Cluyseobacterium scophthainium, particularly, from fungi, can be used in the microorganisms and methods described herein Non-limiting examples of the homologs of FTs in the instant invention are represented by UniProt entries: O30511, P51993, Q11128, G5EFP5, G5EE06, P56434, Q11130, Q11131, P56433, Q8HYJ7, Q8HYJ6, Q17WZ9, Q9ZLI3, D0ISI2, D0I1D1, Q9ZKD7, C7BXF2, E6NNI5, E6NPH4, B6JLN9, C7BZU7, E6NJ21, E6NI06, E6NRI2, E6NSJ6, E6NEQ5, E6NDP7, J0NAV4, and Q9L8S4. Analogues of FTs can be used in the microorganisms and methods described herein.

In some embodiments, FT is selected from α-1,2-fucosyltransferases (FTs) from Helicobacter pylori 26695 (FutC), Bacteroides fragilis (WcfB), or E. coli (such as WbgL, WbgN, and WbwK, for example, wbwK from E. coliO86, wbsJ from E. coli O128, wbgL from E. coli O126, wbiQ from E. coli O127), or futB from H. pylori, futL from H. mustelae, futF from H. bilis, futG from C. jejuni, futN from B. vulgatus ATCC 8482, and wcfB and wcfW from B. fragilis).

In some embodiments, FT is encoded by one of the sequences listed in Table 4 or a variant thereof.

TABLE 4
genes for FT activity.

| Seq. ID NO | Organism | Organism description |
|---|---|---|
| Seq ID NO 26 | Dictyostelium discoideum AX4 | Slime mold |
| Seq ID NO 27 | Homo sapiens | Human enzyme |
| Seq ID NO 28 | Pisum sativa | Plant |
| Seq ID NO 29 | Rhizobium marinum | Bacteria |
| Seq ID NO 30 | Herbaspirillum rubrisubalbicans | Bacteria |
| Seq ID NO 31 | Citrobacter freundii | Bacteria |
| Seq ID NO 32 | Lactobacillus helveticus | Bacteria |
| Seq ID NO 33 | Neocallimastix californiae | Fungi |
| Seq ID NO 34 | Gracilariopsis chorda | Red alga |
| Seq ID NO 35 | Lactobacillus gasseri | Bacteria |
| Seq ID NO 36 | Octopus bimaculoides | Cephalopod |
| Seq ID NO 37 | Chryseobacterium scophthalmum | Bacteria |
| Seq ID NO 38 | Homo sapiens | Human enzyme |
| Seq ID NO 39 | Pisum sativa | Plant |
| Seq ID NO 40 | Neocallimastix californiae | Fungi |

| SEQ ID NO: 26 | |
|---|---|
| 1 | MNDSPIISVV LPFLIKDNDD KSLNYQGINN LIISIDSIIE QTFKEWELIL VDDGSNNEIL |
| 61 | EQLLSKRYST DNRIKFIINK ENKGIVKSLN DAILNHCSPT SKYIARMDSD DISHPTRLQS |
| 121 | QLKYLQSNET IDILGCPIKM FNNNKLIEIL NNNNNNNNIN NNVKELTNII NNEESFKFIQ |
| 181 | HPDKDILMWS MFFNCCIVHP SVIFKRSIFT IEHCYEENNQ FPFIEDYLFW LKSLIMKGLN |
| 241 | ISNIQSSTPL LYLRKHNNSI SFKNIEKQKD STANASCYYL NILFKRFNID SEIIQNSSLS |
| 301 | MKEIIQFFQL SPSSLSKINN ISIELFEFAF KYLELIEKSC TKQQPNYSNS IKDAANEKMG |
| 361 | ELVSLCLSNY PNNQKSSLLW EKWLSRNPTS QLLSLLSNLN VKSSTTIINN NINNNNNNNN |
| 421 | NNNNNNNNNN NNNNNNNNNN SILNFISGIN SNKINTPKSN NNKFKENGIR IICFSKDRAF |
| 481 | QLKEYLRTFF KYLKNDDNGN DKFEIIVDVL FTYSNEKFKN SYQLVIESFP QVNFIKEENF |
| 541 | TDQLINLVQK TNKLEYVMFS VDDILYYNEF NLKEYCLSLN SEPLALGFYM KLNKNITYCH |
| 601 | TCNQDITIPL NSNTISRTEN NFKYLKWNRN DNDCKKDWNY PWDLCSITYR CNDIDSIING |
| 661 | IVKYYGIRNG INHPNRFEFN GNRPIIQKQI YQNKPYCLCL SDHYSPMSVV TINRVQDVYD |
| 721 | NPIYDQTESL DDLDQLLYSN KSLNDEKYKE NSLSLNFKSV HIGELFIS |
| SEQ ID NO: 27 | |
| 1 | MLVVQMPFSF PMAHFILFVF TVSTIFHVQQ RLAKIQAMWE LPVQIPVLAS TSKALGPSQL |
| 61 | RGMWTINAIG RLGNQMGEYA TLYALAKMNG RPAFIPAQMH STLAPIFRIT LPVLHSATAS |
| 121 | RIPWQNYHLN DWMEEEYRHI PGEYVRFTGY PCSWTFYHHL RQEILQEFTL HDHVREEAQK |
| 181 | FLRGLQVNGS RPGTFVGVHV RRGDYVHVMP KVWKGVVADR RYLQQALDWF RARYSSLIFV |
| 241 | VTSNGMAWCR ENIDTSHGDV VFAGDGIEGS PAKDFALLTQ CNHTIMTIGT FGIWAAYLTG |
| 301 | GDTIYLANYT LPDSPFLKIF KPEAAFLPEW TGIAADLSPL LKH |
| SEQ ID NO: 28 | |
| 1 | MNMLIKRVIA IKNPRGDDNN NNKLSDLETL TDKCTTCPLT LMRVMAFFVV SFMLFSVLFS |
| 61 | LSVVLRDPPS DAAISSTTTL FQLNQGLGSD DFDSVELLND KLLGGLLADG FDEKSCLSRY |
| 121 | QSAIFGKGLS GKPSSYLISR LRKYEARHKQ CGPYTESYNK TVKELGSGQF SESVDCKYVV |
| 181 | WISFSGLGNR ILTLVSAFLY ALLTDRVLLV DPGVDMTDLF CEPFPDASWF VPPDFPLNSH |
| 241 | LNNFNQESNQ CHGKILKTKS ITNSTVPSFV YLHLAHDYDD HDKLFFCDEE QLFLQNVPLL |
| 301 | IMKTDNYFIP SLFLMPSFEQ ELNDLFPKKE KVFHFLGRYL LHPTNNVWGL VVRYYDAYLA |
| 361 | KVDERIGIQI RVFDTDPGPF QHVLDQVLAC TLKESILPDV NREQNINSSS GTPKSKAVLI |
| 421 | TSLSSGYFEK VRDMYWEFPT ETGEVVGIYQ PSHEGYQQTQ KQFHNQKAWA EMYLLSLTDV |
| 481 | LVTSSWSTFG YVAQGLGGLK PWILYKPENR TAPNPPCQRA MSMEPCFHAP PFYDCKAKRG |
| 541 | TDTGALVPHV RHCEDMSWGL KLVDN |
| SEQ ID NO: 29 | |
| 1 | MITVKLLGGL GNQMFQFATG RAVARRLGSE LLLDISSFEH YDLRRFELED WAINARVATA |
| 61 | SELARAGVVP SPPRMLTRIS RLLGLAFPAT TFRESSFAYD PGILQVTDPV YLDGYFQSER |
| 121 | YFSDVAGHLR EEFVLRQPTD AKNKAMEALI RDAGPLAVSL HIRRGDYVAN AQTAKYHGVC |
| 181 | SLDYYSAAVD HIAEQVGGGH YFVFSDDLAW VRENLKTTQP MTLVDVNGPD KGAWDMALMT |
| 241 | ACRHHIIANS SFSWWGAWLN PRPDKIIVAP KRWFAGASHD TTDLVPASWI RL |
| SEQ ID NO: 30 | |
| 1 | MIVSRLIGGL GNQMFQYAVG RALAEHMHTP LLLDVSGFVH YDLRRYELDG FNIKAKPASE |
| 61 | EELARLGVKA GVKPSMYERA MRKLGIRREP SILREASFTY DARIETVEAP LYLDGYWQSQ |
| 121 | RYFAAIRPQL LQEFSLKDSW GSANDALAEQ IGLAGDGAVS LHVRRGDYVN NAQTAQYHGV |
| 181 | CSLDYYRQAV AYIVARVAAP HFFVFSDDHA WVSANLDTGC PTTFVQTNSP DQGIFDMALM |
| 241 | KTCRHHIIAN SSFSWWGAWL NANDEKIVVA PQRWFNEASK DTSDLIPAGW VRL |
| SEQ ID NO: 31 | |
| 1 | MQVNRVYVRP MGGLGNQLFQ YAVAYGVARK HSAQLIIDTR FFDNYELHGG FRLYNLNISV |
| 61 | SEMTNADLKK FPEWKCKLLS KFPQVTRFFN EYIYDKVGDL NEIKSNDAML LGYWQNETNF |
| 121 | HQYKNELVTI FKPKIISEND NKKAESILAT NSVVIHIRRG DYINNPIAYK HHGVCSLNYY |
| 181 | KQAINEMKKN TKNIFFYIFS DDIEWCRENT TPLFSEYDSF SFVRGETQEV DMWLMSCGKY |
| 241 | HIIANSSFSW WGAFLSTNPD QIVIAPTPWF DITQKYTGDP SLPQWIKIDK Y |
| SEQ ID NO: 32 | |
| 1 | MLYMRLKGRL GNQLFIYAFA RELVYKYNQQ VLLYDRKDEK DSMWYSHLDN YPLNTNVHFT |
| 61 | SNRRDMKIGN FKSKLRFIYD RVAIRIMPPR ERYNFQIRNL KKNEKNSLFL LNIDGYAPLPK |
| 121 | KINDGTFFDG YFQSPKYFNN IREELIKELN PVHTYSEEEK KFINKIKNTE SVCVTIRLGD |
| 181 | YINNSTHQVC SKEFYLNAMD KLKKIYPDCT FFIFSDEVDK AQQIFDFKYP VIYDSGKMQD |
| 241 | YVSLHVMSMC KHFIISNSSF SWWAQYLSTN PQKIVIAPDK WYAQDVPCDI YEDNWVLMKG |
| 301 | K |
| SEQ ID NO: 33 | |
| 1 | MKILIFSVSF SFFYLLHLFF ILYYIISKAS KEIRIVKLCG GLGNQMFQYA YGKSLEHKLQ |
| 61 | EKVLFDVSWY KYLNKKKNEK LTKREYGLGI FNLKISFPTK KQLKKCNNKT FEKKSYIYDE |
| 121 | ELLQNKGSSY YVGYFQNEKY FKDIKDNIKK IYFFPKIHDT DKFNQQWINK IKNVKNSVFI |
| 181 | HIRRADYTYL DGWVLSNDYY KKAIEYIKKN VENPTFFIFC YQCKDYVEEQ FKLDDTIQFI |
| 241 | GETNSINNEN WKDMVLMKEC KYAIIANSSF SWWAAWLGRA NEEGIVIAPS PFIKNNDEII |
| 301 | CDNWIKINSN NSS |
| SEQ ID NO: 34 | |
| 1 | MGLRERLHSV WFLWFVAFSI IAVGFLSRSV RTPSVPQQLK STVLVTLSGR LGNQLFQVAA |
| 61 | SEFITARIKP QKVLFLRNNY SAETDFSQGV FRDLKHVNSV SEACRGLRRN YYSHKRMSCS |
| 121 | HVRRNQLKGE CLIVEGLFQC PHFANAGSSL VRSLFESSLI ASKAEETYRS YAAVSPASPV |
| 181 | VAIHIRRGDY TKRFNRNFLE PLPMKYYIRA TKFMPKNAIY LVFSDDTAWC KSNLPELFRK |
| 241 | IPHSRLIFVK ETDASISLAL MSLADHFIIA NSTFSWWAAF LRRFEKKIVV SPKNWFGDRV |
| 301 | TEKNKIYPRK WIRV |
| SEQ ID NO: 35 | |
| 1 | MLYVEMDGRC GNQLFHYAVA RYIQLAIGNK EKLCLNFNKI FEKKDENNGW IDYLKDFKTV |
| 61 | PYSYYSKSGT ILKNESNFIQ KIAIGLKAIQ IKSLTKKSRQ EQADKAEVGQ RTLNKLGVYW |
| 121 | VREGVNQIYP YKNNKILVSG ICESNFIYEI QEQLQKELIP VTPVSSLNKS LLEKIDNCNS |
| 181 | VCISVRRGDF FNNKNAKKYG VCSPEYYIRA KKYFDKKRLE NTVYFCFSDD IEWCKENLKF |
| 241 | TDKNVIFVSQ EMPVYETLRL MSRCKHFILS NSTFSWWGQF LSEYKDKIVV SPARWNNDGY |
| 301 | DTNLIDKNWI LIDA |
| SEQ ID NO: 36 | |
| 1 | MLLPCWLYHC YCFYHDVAAV GVFHSAFCVK LLIFFIVFFL GVIIFHYLDI LGVIYTINYL |
| 61 | VHRQQDDTKV LCPKFVGGLG NQMFQYASLY GIAKSKNMTL LIDAECELNQ LFSISAVTLP |
| 121 | HVACWFLKTR TDYRPCAFNK DTMNFSADQN YQMQGYLQSW QYFHRAEPAL RQIFKFKAAI |
| 181 | REKAESILKQ AIEVHQKQVR NQALTFIAIH IRRGDITKDN FKTYGYNTAS LDYIRRAMQY |
| 241 | FSERYHRILF LVCTNDMEWA KRYLHKKNVY FVENQPREVD MALMASCNHT IMYVGSFGWW |
| 301 | SAWLANGEVV YYRYPASRGS KLRKAFSKEM TDYYYPKWKP ML |
| SEQ ID NO: 37 | |
| 1 | MVAVELIGGL GNQMFQYATA RALSLHRDDS LLLDSRLFDN YKLHSYCLNH FNIGAAVVKN |
| 61 | DLSLKTPGFS KRVVDKLLQK IDALTLQNKI FNTYQEKNLL FDDSLFRNSK KNIYLKGYTQ |
| 121 | SEKYFAKYED QLRKDFEIVT PLKKETTDLL KIIEAENSVS LHIRRGDYIS NPAANAVHGT |
| 181 | CDLNYYHRAI EIIKEKIEHP IFFIFSDDID WAKENLKLEN TTYFVDFNDA STNYEDLKLM |
| 241 | SACKNNITAN SSFSWWGAWL NANKSKIVIA PSKWFNTDVL NSQDIIPESW MKI |
| SEQ ID NO 38 | |
| 1 | MLAKIQAMWE LPVQIPVLAS TSKALGPSQL RGMWTINAIG RLGNQMGEYA TLYALAKMNG |
| 61 | RPAFIPAQMH STLAPIFRIT LPVLHSATAS RIPWQNYHLN DWMEEEYRHI PGEYVRFTGY |
| 121 | PCSWTFYHHL RQEILQEFTL HDHVREEAQK FLRGLQVNGS RPGTFVGVHV RRGDYVHVMP |
| 181 | KVWKGVVADR RYLQQALDWF RARYSSLIFV VTSNGMAWCR ENIDTSHGDV VFAGDGIEGS |
| 241 | PAKDFALLTQ CNHTIMTIGT FGIWAAYLTG GDTIYLANYT LPDSPFLKIF KPEAAFLPEW |
| 301 | TGIAADLSPL LKH |
| SEQ ID NO: 39 | |
| 1 | MGLGSDDFDS VELLNDKLLG GLLADGFDEK SCLSRYQSAI FGKGLSGKPS SYLISRLRKY |
| 61 | EARHKQCGPY TESYNKTVKE LGSGQFSESV DCKYVVWISF SGLGNRILTL VSAFLYALLT |
| 121 | DRVLLVDPGV DMTDLFCEPF PDASWFWPPD FPLNSHLNNF NQESNQCHGK ILKTKSITNS |
| 181 | TVPSEVYLHL AHDYDDHDKL FFCDEEQLFL QNVPLLIMKT DNYFIPSLFL MPSFEQELND |
| 241 | LFPKKEKVFH FLGRYLLHPT NNVWGLVVRY YDAYLAKVDE RIGIQIRVFD TDPGPFQHVL |
| 301 | DQVLACTLKE SILPDVNREQ NINSSSGIPK SKAVLITSLS SGYFEKVRDM YWEFPTETGE |
| 361 | VVGIYQPSHE GYQQTQKQFH NQKAWAEMYL LSLTDVLVTS SWSTFGYVAQ GLGGLKPWIL |
| 421 | YKPENRTAPN PPCQRAMSME PCFHAPPFYD CKAKRGTDTG ALVPHVRHCE DMSWGLKLVD |
| 481 | N |
| SEQ ID NO: 40 | |
| 1 | MKLCGGLGNQ MFQYAYGKSL EHKLQEKVLF DVSWYKYLNK KKNEKLTKRE YGLGIFNLKI |
| 61 | SFPTKKQLKK CNNKTFEKKS YIYDEELLQN KGSSYYVGYF QNEKYFKDIK DNIKKIYTFP |
| 121 | KIHDTDKFNQ QWINKIKNVK NSVFIHIRRA DYIYLDGWVL SMDYYKKAIE YIKKNVENPT |
| 181 | FFIFCYQCKD YVEEQFKLDD TIQFIGETNS INNENWKDMV LMKECKYAII ANSSFSWWAA |
| 241 | WLGRANEEGI VIAPSPFIKN NDEIICDNWI KINSNNSS |
| alpha-1,2-fucosyltransferase (WbgL) [Escherichia coli] | |
| SEQ ID NO: 47 | |
| 1 | MRSIIRLQGG LGNQLFQFSF GYALSKINGT PLYFDISHYA ENDDHGGYRL NNLQIPEEYL |
| 61 | QYYTPKINNI YKFLVRGSRL YPEIFLFLGF CNEFHAYGYD FEYIAQKWKS KKYIGYWQSE |
| 121 | HFFHKHILDL KEFFIPKNVS EQANLLAAKI LESQSSLSIH IRRGDYIKNK TATLTHGVCS |
| 181 | LEYYKKALNK IRDLAMIRDV FIFSDDIFWC KENIETLLSK KYNIYYSEDL SQEEDLWLMS |
| 241 | LANHHIIANS SFSWWGAYLG TSASQIVIYP TPWYDITPKN TYIPIVNHWI NVDKHSSC |
| futC_Hp26695 from H. pylori | |
| SEQ ID NO: 48 | |
| 1 | MAFKVVQICG GLGNQMFQYA FAKSLQKHLN TPVLLDITSF DWSNRKMQLE LFPIDLPYAS |
| 61 | AKEIAIAKMQ HLPKLVRDTL KCMGFDRVSQ EIVFEYEPGL LKPSRLTYFY GYFQDPRYFD |
| 121 | AISPLIKQTF TLPPPENGNN KKKEEEYHRK LALILAAKNS VFVHVRRGDY VGIGCQLGID |
| 181 | YQKKALEYIA KRVPNMELFV FCEDLKFTQN LDLGYPFMDM TTRDKFEEAY WDMLLMQSCK |
| 241 | HGIIANSTYS WWAAYLINNP EKIIIGPKHW LFGHENILCK EWVKIESHFE VKSKKYNA |
| Putative fucosyl transferase from Bacteroides fragilis | |
| SEQ ID NO: 49 | |
| 1 | MLYVILRGRL GNNLFQIATA ASLTQNFIFC TVNKDQERQV LLYKDSFFKN IKVMKGVPDG |
| 61 | IPYYKEPFHE FSRIPYEEGK DLIIDGYFQS EKYFKRSVVL DLYRITDELR KKIWNICGNI |
| 121 | LEKGETVSIH VRRGDYLKLP HALPFCGKSY YKNAIQYIGE DKIFIICSDD IDWCKKNFIG |
| 181 | KRYYFIENTT PLLDLYIQSL CTHNIISNSS FSWWGAWLNE NSNKIVIAPQ MWTGISVKLG |
| 241 | VSDLLPVSWV RLPNNYTLGR YCFALYKVVE DYLLNILRLI WKRKKNM |
| wbgN from E. coli | |
| SEQ ID NO: 50 | |
| 1 | MSIVVARLAG GLGNQMFQYA KGYAESVERN SSLKLDLRGY KNYTLHGGFR LDKLNIDNTF |
| 61 | VMSKKEMCIF PNFIVRAINK FPKLSLCSKR FESEQYSKKI NGSMKGSVEF IGFWQNERYF |
| 121 | LEHKEKLREI FTPININLDA KELSDVIRCT NSVSVHIRRG DYVSNVEALK IHGLCTERYY |
| 181 | IDSIRYLKER FNNLVFFVFS DDIEWCKKYK NEIFSRSDDV KFIEGNTQEV DMWLMSNAKY |
| 241 | HIIANSSFSW WGAWLKNYDL GITIAPTPWF EREELNSFDP CPEKWVRIEK |
| wbwK from E. coli | |
| SEQ ID NO: 51 | |
| 1 | MYSCLSGGLG NQMFQYAAAY ILQRKLKQRS LVLDDSYFLD CSNRDTRRRF ELNQFNICYD |
| 61 | RLTTSKEKKE ISIIRHVNRY RLPLFVTNSI FGVLLKKNYL PEAKFYEFLN NCKLQVKNGY |
| 121 | CLFSYFQDAT LIDSHRDMIL PLFQINEDLL HLCNDLHIYK KVICENANTT SLHIRRGDYI |
| 181 | TNPHASKFHG VLPMDYYEKA IRYIEDVQGE QVIIVESDDV KWAENTFANQ PNYYVVNNSE |
| 241 | CEYSAIDMFL MSKCKNNIIA NSTYSWWGAW LNTFEDKIVV SPRKWFAGNN KSKLTMDSWI |
| 301 | NL |
| wbsJ from E. coli | |
| SEQ ID NO: 52 | |
| 1 | MEVKIIGGLG NQMFQYATAF AIAKRTHQNL TVDISDAVKY KTHPLRLVEL SCSSEFVKKA |
| 61 | WPFEKYLFSE KIPHFMKKGM FRKHYVEKSL EYDPDIDTKS INKKIVGYFQ TEKYFKEFRH |
| 121 | ELIKEFQPKT KFNSYQNELL NLIKENDTCS LHIRRGDYVS SKIANETHGT CSEKYFERAI |
| 181 | DYLMNKGVIN KKTLLFIFSD DIKWCRENIF FNNQICFVQG DAYHVELDML LMSKCKNNII |
| 241 | SNSSFSWWAA WLNENKNKTV IAPSKWFKKD IKHDIIPESW VKL |
| wbiQ from E. coli | |
| SEQ ID NO: 53 | |
| 1 | MVMMYCCLSG GLGNQMFQYA AAYILKQHFP DTILVLDDSY YFNQPQKDTI RHLELDQFKI |
| 61 | IFDRFSSKDE KVKINRLRKH KKIPLINSFL QFTAIKLCNK YSLNDASYYN PESIKNIDVA |
| 121 | CLFSFYQDSK LLNEHRDLIL PLFEIRDDLR VLCHNLQIYS LITDSKNITS IHVRRGDYVN |
| 181 | NKHAAKFHGT LSMDYYISAM EYIESECGSQ TFIIFTDDVI WAKEKFSKYS NCLVADADEN |
| 241 | KFSVIDMYLM SLCNNNIIAN STYSWWGAWL NRSEDKLVIA PKQWYISGNE CSLKNENWIA |
| 301 | M |
| futB from H. Pylori | |
| SEQ ID NO: 54 | |
| 1 | MVFQPLLDAF IESASIEKMV SKSPPPPLKI AVANWWGDEE IKEFKKSVLY FILSQRYAIT |
| 61 | LHQNPNESSD LVFSNPLGAA RKILSYQNTK RVFYTGENES PNFNLFDYAI GFDELDFNDR |
| 121 | YLRMPLYYAH LHYEAELVND TTAPYKLKDN SLYALKKPSH HFKENHPNLC AVVNDESDLL |
| 181 | KRGFASFVAS NANAPMRNAF YDALNSIEPV TGGGSVRNTL GYKVGNKSEF LSQYKFNLCF |
| 241 | ENSQGYGYVT EKILDAYFSH TIPIYWGSPS VAKDFNPKSF VNVHDFNNFD EAIDYIKYLH |
| 301 | THPNAYLDML YENPLNTLDG KAYFYQDLSF KKILDFFKTI LENDTIYHNN PFIFYRDLHE |
| 361 | PLISIDDLRV NYDDLRVNYD DLRVNYDDLR VNYDDLRVNY DDLRVNYDDL RVNYDDLRVN |
| 421 | YDDLRVNYDD LRVNYDRLLQ NASPLLELSQ NTTFKIYRKA YQKSLPLLRT IRRWVKK |
| full from H. mustelae | |
| SEQ ID NO: 55 | |
| 1 | MDFKIVQVHG GLGNQMFQYA FAKSLQTHLN IPVLLDTTWF DYGNRELGLH LFPIDLQCAS |
| 61 | AQQIAAAHMQ NLPRLVRGAL RRMGLGRVSK EIVFEYMPEL FEPSRIAYFH GYPQDPRYFE |
| 121 | DISPLIKQTF TLPHPTEHAE QYSRKLSQIL AAKNSVFVHI RRGDYMRLGW QLDISYQLRA |
| 181 | IAYMAKRVQN LELFLFCEDL EFVQNLDLGY PFVDMTTRDG AAHWDMMLMQ SCKHGIITNS |
| 241 | TYSWWAAYLI KNPEKIIIGP SHWIYGNENI LCKDWVKIES QFETKS |
| futF from H. bilis | |
| SEQ ID NO: 56 | |
| 1 | MEDNLIIVRV DGGIASQIGF VALGKAFEEK GYQVKYDLSW FETSGKGFYN TINGYDRYDD |
| 61 | LTFDMPKAFP QLEMKIASED EVKRYNKLYF IDDEKVITHK PPLYVGGYLG RHYDIYFARH |
| 121 | FATYFSPKEI EQKDAPFYIL LQEILNTQSC GIHIRRGDLS QNHIVYGEPT SLTYFERVIQ |
| 181 | LVAQMNSKSV FYLFSDDVAW VREHIAPLLK DKQFKICDIN TPEQGYLDLY LLSRCKVIVA |
| 241 | SHGSLGAYAK ILAPHNPLLI APRVRNVFFE MENVMLVNWG AKLQITQPCN NVITPPPHCQ |
| 301 | NLTLRYRLFL YLYNRLRSKL LRKGVIQ |
| futG from H. jejuni | |
| SEQ ID NO: 57 | |
| 1 | MLESNFVIIR VDGGIVSQLY FFAIGKLFEK KGYKVKYDIT WFEEEGLGFY NINKGYDKTY |
| 61 | NINWDIPKIF PNISIEIASK SEIDQYKKFR VDSELVLEYQ PPLYVVGYNS KCDIVEICRE |
| 121 | IREFFNPLEL LSDNKIKFLA NEIKRNRSCG VHVRRGDLSQ EHVVYGKPTS VDYFFKCINI |
| 181 | VRSMYSDAKF YFFSDDNKWV KDNIAPHIEN LDYFICDINT PEKGYLDLYF LSLCKIIIGS |
| 241 | HGSMGLGAKL LSQEETLFIT PKYNYMLFSM SNIMMINFEP KNMEPFNPKI KKIKYKILIK |
| 301 | IYYYTRQILL RKFLIKGSD |
| futN from B. vulgatus | |
| SEQ ID NO: 58 | |
| 1 | MRLIKVTGGL GNQMFIYAFY LRMKKYYPKV RIDLSDMMHY KVHYGYEMHR VFNLPHTEFC |
| 61 | INQPLKKVIE FLFFKKIYER KQAPNSLRAF EKKYFWPLLY FKGFYQSERF FADIKDEVRE |
| 121 | SFTFDKNKAN SRSLNMLEIL DKDENAVSLH IRRGDYLQPK HWATTGSVCQ LPYYQNAIAE |
| 181 | MSRRVASPSY YITSDDIAWV KENLPLQNAV YIDWNIDEDS WQDMMLMSHC KHHIICNSTF |
| 241 | SWWGAWLNPN MDKTVIVPSR WFQHSEAPDI YPTGWIKVPV S |
| wcfW from B. fragilis | |
| SEQ ID NO: 59 | |
| 1 | MIVSSLRGGL GNQMFIYAMV KAMALRNNVP FAFNLTTDFA NDEVYKRKLL LSYFALDLPE |
| 61 | NKKLTFDFSY GNYYRRLSRN LGCHILHPSY RYICEERPPH FESRLISSKI TNATLEGYWQ |
| 121 | SEKYFLDYKQ EIKEDFNIQK KLEYTSYLEL EEIKLLDKNA IMIGVRRYQE SDVAPGGVLE |
| 181 | DDYYKCAMDI MASKVTSPVF FCFSQDLEWV EKHLAGKYPV RLISKKEDDS GTIDDMFLMM |
| 241 | HFRNYIISNS SFYWWGAWLS KYDDKLVIAP GNFINKDSVP ESWFKLNVR |
| futA | |
| SEQ ID NO: 63 | |
| 1 | MGFQPLLDAF IESASIEKMA SKSPPPPLKI AVANWWGDEE IKEFKKSVLY FILSQRYAIT |
| 61 | LHQNPNEFSD LVFSNPLGAA RKILSYQNTK RVFYTGENES PNFNLFDYAI GFDELDFNDR |
| 121 | YLRMPLYYAH LHYKAELVND TTAPYKLKDN SLYALKKPSH HFKENHPNLC AVVNDESDLL |
| 181 | KRGFASFVAS NANAPMRNAF YDALNSIEPV TGGGSVRNTL GYKVGNKSEF LSQYKFNLCF |
| 241 | ENSQGYGYVT EKILDAYFSH TIPIYWGSPS VAKDFNPKSF VNVHDFNNFD EAIDYIKYLH |
| 301 | THPNAYLDML YENPLNTLDG KAYFYQDLSF KKILDFFKTI LENDTIYHKF STSFMWEYDL |
| 361 | MKPLVSIDDL RVNYDDLRVN YDRLLQNASP LLELSQNTTF KIYRKAYQKS LPLLRAVRKL |
| 421 | VKKLGL |
| futD | |
| SEQ ID NO: 64 | |
| 1 | MDKQILNMRV LDWWTEDNEQ NFYDNIFIRL LQRKYEVVYS DTPDFVLCGP FGYKHLEYRG |
| 61 | VRIFCTGENV RPDFNLVDYA ISFDYAVFGD RHLRTPLMFL CDDYVEDMQK VLNSRAHLIK |
| 121 | SKIKTCSFIA SNNYMTEMRD SFFEALCTYK KVDSGGKWKN NIGVYVDDKI EWLKSYKFNI |
| 181 | CFENDSSPGY LTEKLFDAFM GGCVPIYWGD TSLRCKVDNE CGNLIETQEI GYHLNLEQTK |
| 241 | KEVDFVYNGG GYGMFDTRIP NIPAYLFDYK INPKAFINAH DFPTFKELID EIKRIDNDEQ |
| 301 | AFKDMLNEPV FLNNFNPKEF YSQKTFHFLD YIVSQGPVCA KRIGRGSRLQ RKENIMRMFP |
| 361 | YDTDSVLIPN FMSYCVKHKK IIDRVRRVCG FPRDIMRTIR GK |
| futE | |
| SEQ ID NO: 65 | |
| 1 | MQKQQVKMRV LDWWNKDCEE NFYNNFFIQI LQKKYDVVYS DKPDFILYGP CGYEHLKYDC |
| 61 | VRIFYTAENI RPDYNIADYS IDYDYIKFGD RHLRLPYMFW VFCDEMRQKE MDNRISLLDK |
| 121 | KEKFCGFMVS NNALTDKRDM FFEALNKYKR VDSGGRWKNN IGGWDDKKIE WLKSYKFNLC |
| 181 | FENSSYPGYL TEKLFDAFLA GCVPIYWGDT SLRVHKNTCA DSKNSENINN RGGGGNDTFD |
| 241 | MRIPNISHSL IDYEINPKAF INAHNFPTFK DLIDEIKRID NDSYAFESIL REPIFINNFS |
| 301 | PYEFYTEQIS AFLDHIIMQG ANDARRCGDG YWLRTHLEFR RISAKYWNLP SDFLHYCFKY |
| 361 | RKIIQGVRDI SEYPRNFMRF LRRK |
| FutII | |
| SEQ ID NO: 66 | |
| 1 | MAQNLQTPQD SKTKKRIYFC DGAVKGKIPA ILSRHYDIEI TPHNPDYVFY SVMGNEHINY |
| 61 | DCIRIFSTGE NVRADFNFCD YAIGFDYMQF EDRYLRYPFY LHYKEAMEKA RNKHLHITPQ |
| 121 | TLENKKRFCT FVVSNGKADS IRSQFFDKLM QYKHIDSGGK YKNNIGAPVA DKLAFLSEGK |
| 181 | ENIAFENSSA NGYTTEKLIE AFAAGTIPLY WGDESVSLPL DSSGGGVNPK SFVRLNDFAS |
| 241 | FEEAIAYIEF LDTHNDAYLA ILREETFLDS NHEAIFDKKL ESFLLHIFNQ PLEKAYRRGF |
| 301 | GQWRCNIEKR YKKYQRIRSL TNTCVNIIKN PIRRIKKLFK |
| FutJ | |
| SEQ ID NO: 67 | |
| 1 | MKDDLVILHP DGGIASQIAF VALGLAFEQK GAKVKYDLSW FAEGAKGFWN PSNGYDKVYD |
| 61 | ITWDISKAFP ALHIEIANEE EIERYKSKYL IDNDRVIDYA PPLYCYGYKG RIFHYLYAPF |
| 121 | FAQSFAPKEA QDSHTPFAAL LQEIESSPSP CGVHIRRGDL SQPHIVYGNP TSNEYFAKSI |
| 181 | ELMCLLHPQS SFYLFSDDLA FVKEQIVPLL KGKTYRICDV NNPSQGYLDL YLLSRCRNII |
| 241 | GSQGSMGEFA KVLSPHNPLL ITPRYRNIFK EVENVMCVNW GESVQHPPLV CSAPPPLVSQ |
| 301 | LKRNAPLNSR LYKEKDNASA |
| FutK | |
| SEQ ID NO: 68 | |
| 1 | MNQGCTKTHK PTKKVYFCDG AVKGKIVALL EQHYELILTN KDPDYIFYSC MGFEHLNYNK |
| 61 | VRIFATGENL RADFNFCDYA IGYDYTHFED RYLRYPLYLH CESDMQKAMN KHLHITPETL |
| 121 | QNKSRFCTFV VSNGKADEIR TQFFDFLSQY NRVDSGGRYK NNIGNPVVDK YAFLKEGKFN |
| 181 | IAFENSSTNG YITEKLIQAF AAHTIPIYWG DERISLPLDK MGGGINPKSF INMHKYESYK |
| 241 | EVLETIYFLD THDEAYLSML SEPVFLDKNH QKIFDEKLEN FLLHIFNQPL EKAYRRGFGQ |
| 301 | WRCNIEKRYK KAQKARQIVN NFANIFQIPL RTLKKYLLSI YLSATSKSFV FFTKERTSK |
| FutM | |
| SEQ ID NO: 69 | |
| 1 | MCDCLSIILL VKMKKIYLKF VDFWDGFDTI SNFIVDALSI QYEVVLSNEP DYLFYSCFGT |
| 61 | SHLEYDCIKI MFIGENIVPD FNVCDYAIGF NYIDFGDRYL RLPLYAIYDG FSNLQNKKID |
| 121 | VNKALDRKFC SIVVSNNKWA DPIRETFFKL LSSYKKVDSG GRAWNNIGGP VDNKLDFISQ |
| 181 | YKFNIAFENS RVLGYTTEKI MEPMQVNSIP VYWGNPLVGK DFNVDSFVNA HDFDSLERLV |
| 241 | EYIIELDSSK DKYLEMLEKP WLLDKTYLDW KQLLLNFINN IMMKSYKDAK YLVNYGHAGK |
| 301 | YRNEQRFWGR CERKFKLQRI IEYYSQLFDR K |

In some embodiments, the nucleic acids encoding an enzyme sequence include a targeting sequence, such as for localization to a specific cellular organelle. In some embodiments, such sequence is removed from the nucleic acid prior to providing it as a heterologous sequence through genetic engineering into a microorganism. For example, the targeting sequence of SEQ ID Nos. 27, 28, 33, 38, 39 or 40 can be removed before the encoded FT is genetic engineered for expression in a microorganism.

Other FTs that can be used for HMO production in a microorganism, include, but are not limited to, UniProt entries O30511, P51993, Q11128, G5EFP5, G5EE06, P56434, Q11130, Q11131, P56433, Q8HYJ7, Q8HYJ6, Q17WZ9, Q9ZLI3, D0ISI2, D0ITD1, Q9ZKD7, C7BXT2, E6NNI5, E6NPH4, B6JLN9, C7BZU7, E6NJ21, E6NI06, E6NRI2, E6NSJ6, E6NEQ5, F6NDP7, J0NAV4, and Q9L8S4. Analogues and homologs of FTs also can be used in the microorganisms and methods described herein.

The UniProt entries listed herein are incorporated by reference in their entireties. Additional homologs of FTs are known in the art and such embodiments are envisioned for use with the engineered microorganisms and methods here. For example, the homologs of FTs have at least 60%, 65%, 70%, 75%, 80%, 85%, 90%, 95%, 96%, 97%, 98%, 99% or greater than 99% sequence identity to SEQ ID NOs: 26-40.

In some embodiments, an HMO such as 2′-FL, can be synthesized using so-called salvage pathway enzymes. For example, for 2′-FL, a microorganism can utilize lactose and fucose substrates to synthesize 2′-FL, using an enzyme to convert fucose and ATP to fucose-1-phosphate and an enzyme to convert the fucose-1-phosphate and GTP to GDP-fucose, which then can be converted by a fucosyl transferase (FT) to 2′-FL. In some embodiments, a bifunctional fucokinase/L-fucose-1-P-guanylyltransferase (FKP) enzyme such as fkp from Bacteroides fragilis performs the two enzymatic steps from fucose to GDP-fucose and then a FT coverts the GDP-fucose to 2′-FL. In some embodiments, the kfp is from B. fragilis 9343, B. thetaiotaomircon or B. ovatus. For example, the FT may be fu12 from Heliobacter pylori or any of the FTs described herein. In some embodiments, lactose is supplied exogenously to the microorganism and a transporter such as Lac 12, CDT-1, CDT-2 or a variants or homolog, thereof imports the lactose intracellularly for conversion to the HMO.

| Bifunctional fucokinase/L-fucose-1-P-guanylyltransferase (FKT) | |
|---|---|
| [Bacteroides fragilis] | |
| SEQ ID NO: 70 | |
| 1 | MQKLLSLPPN LVQSFHELER VNRTDWFCTS DPVGKKLGSG GGTSWLLEEC YNEYSDGATF |
| 61 | GEWLEKEKRI LLHAGGQSRR LPGYAPSGKI LTPVPVFRWE RGQHLGQNLL SLQLPLYEKI |
| 121 | MSLAPDKLHT LIASGDVYIR SEKPLQSIPE ADVVCYGLWV DPSLATHHGV FASDRKHPEQ |
| 181 | LDFMLQKPSL AELESLSKTH LFLMDIGIWL LSDRAVEILI KRSHKESSEE LKYYDLYSDF |
| 241 | GLALGTHPRI EDEEVNTLSV AILPLPGGEF YHYGTSKELI SSTLSVQNKV YDQRRIMHRK |
| 301 | VKPNPAMFVQ NAVVRIPLCA ENADLWIENS HIGPKWKIAS RHIITGVPEN DWSLAVPAGV |
| 361 | CVDVVPMGDK GFVARPYGLD DVFKGDLRDS KTTLTGIPFG EWMSKRGLSY TDLKGRTDDL |
| 421 | QAASVFPMVN SVEELGLVLR WMLSEPELEE GKNIWLRSER FSADEISAGA NLKRLYAQRE |
| 481 | EFRKGNWKAL AVNHEKSVFY QLDLADAAED FVRLGLDMPE LLPGDALQMS RIHNRMLRAR |
| 541 | ILKLDGKDYR PEEQAAFDLL RDGLLDGISN RKSTPKLDVY SDQIVWGRSP VRIDMAGGWT |
| 601 | DTPPYSLYSG GNVVNLAIEL NGQPPLQVYV KPCKDFHIVL RSIDMGAMEI VSTFDELQDY |
| 661 | KKIGSPFSIP KAASLAGFAE PAVSAVSYAS LEEQLKDFGA GIEVTLLAAI PAGSGLGTSS |
| 721 | ILASTVLGAI NDFCGLAWDK NEICQRTLVL EQLLTTGGGW QDQYGGVLQG VKLLQTEAGF |
| 781 | AQSPLVRWLP DHLFTHPEYK DCHLLYYTGI TRTAKGILAE IVSSMFLNSS LHLNLLSEMK |
| 841 | AHALDMNEAI QRGSFVEFGR LVGKTWEQNK ALDSGTNPPA VEAIIDLIKD YTLGYKLPGA |
| 901 | GGGGYLYMVA KDPQAAVRIR KILTENAPNP RARFVEMTLS DKGFQVSRS |
| Bifunctional fucokinase/L-fucose-1-P-guanylyltransferase (FKP) | |
| [Bacteroides thetaiotaomicron] | |
| SEQ ID NO: 71 | |
| 1 | MPEPICCFLL CRHSAIAGIQ SCYKPINTDS TMQKLLSLPP NLIDSFHQLE EVNHTDWFCT |
| 61 | SDPVGSKLGS GGGTTWLLQA CHQAFAPEET FSKWIGNEKK ILLHAGGQSR RLPGYAPSGK |
| 121 | ILTPIPVFSW ERGQKLGQNL LSLQLPLYER IMKQAPKGLN TLIASGDVYI RSEKPLQDIP |
| 181 | EVDVVCYGLW VNPSLATHHG VFVSDRKKPE VLDFMLQKPS LEELEGLSKT HLFLMDIGIV |
| 241 | ILSDRAVEVL MKRSLKEGTN DISYYDLYSD YGLALGEHPQ TTDDEVNKLS VAILPLPGGE |
| 301 | FYHFGTSREL ISSTLAIQDK VRDQRRIMHR KVKPNPAIFI QNSFTQVKLS AENANLWIEN |
| 361 | SHVGEGWKLG SRQIITGVPE NHWNINLPDG VCIDIVPMGD AAFVARPYGL DDVFKGDLSN |
| 421 | DSTTYLGNSF TQWMKEREIG LEDIKGRTDD LQAAPVFPVT TSIEELGILI RWMTAEPQLK |
| 481 | EGKELWLRAE KLSADEISAQ ANLERLYAQR SAFRRDNWKG LSANYEKSVF YQLDLQDAAN |
| 541 | EFVRLNLDVP AVLKEDAAPM VRIHNRMLRA RILKLQGNEG CKGEEQAAFQ LLRDGLLEAV |
| 601 | AGKKNYPKLN VYSDQIVWGR SPVRIDVAGG WTDTPPYSLY SGGSVVNLAI ELNGQPPLQV |
| 661 | YVKPCHEFHI VLRSIDMGAV EVIRSYEELQ DYKKVGSPFS IPKAALTLAG FAPLFAAESH |
| 721 | ASLEEHLKAF GSGLEITLLA AIPAGSGLGT SSILASTVLG AINDFCGLAW DRNDICNYTL |
| 781 | VLEQLLTTGG GWQDQYGGVF PGVKLLQSES GFEQHPLVRW LPDQLFVQPE VRDCHLLYYT |
| 841 | GITRTAKGIL AEIVSSMFLN SGKHLSLLAE MKAHAMDMSE AILRGNFETF GNLVGKSWIQ |
| 901 | NQALDSGTNP PAVAAIIEQI KDYTLGYKLP GAGGGGYLYM VAKDPQAAGC IRRILTEQAP |
| 961 | NPRARFVEMT LSDKGLQVSR S |
| Bifunctional fucokinase/L-fucose-1-P-guanylyltransferase (FKP) | |
| [Bacteroides ovatus] | |
| SEQ ID NO: 72 | |
| 1 | MQKLLSLPPN LIHCFHELEE VNHTDWFCTS DPIGSKLGSG GGTTWLLQAC HQAFAPQESF |
| 61 | SNWIGHEKRI LLHAGGQSRR LPSYGPSGKI LTPIPIFSWE RGQKLGQNLL SLQLPLYERI |
| 121 | MNQAPAGLNT LIASGDVYIR SEKPLQDIPN ADVVCYGLWV NPSLATHHGV FVSDRKKPEV |
| 181 | LDFMLQKPSL EELEGLSKTH LFLMDIGIWI LSDRAIEVLM KRSLKEGTKD ITYYDLYSDY |
| 241 | GLTLGEHPKT KDEEINQLSV AILPLPGGEF YHYGTSHELI SSTLAIQDKV RDQRRIMHRK |
| 301 | VKPNPAIFIQ NSITQVSLSA DNANLWIENS QVGKEWKLGS RQIITGVPEN QWSINLPDGV |
| 361 | CIDIIPIGEN EFVARPYGLD DVFKGALDKI TTTYLNVPFT RWMEDRGITW EDIKGRTDDL |
| 421 | QSASIFPKVA SVEDLGILVR WMTSEPQLEE GKKLWLKAEK VSADEISASA NLKRLYEQRN |
| 481 | AFRKENWRGL AANYEKSVFY QLDLLDAANE FVRFNLDMPD VLKEDAAPML RIHNRMLRAR |
| 541 | IMKLREDKDC AKEEQAAFQL LRDGLLGVMS ERKSHPILNV YSDQIVWGRS PVRIDVAGGW |
| 601 | TDTPPYSLYS GGSVVNLAIE LNGQPPLQVY VKPCKEYHIT LRSIDMGAME VIRNYEELQD |
| 661 | YKKVGSPFSI PKAALTLAGF APAFSTESYP SLAKQLEDFG SGIEITLLAA IPAGSGLGTS |
| 721 | SILASTVLGA INDFCGLAWD KNDICSYTLV LEQLLTTGGG WQDQYGGVFS GIKLLQSEAG |
| 781 | FEQNPLVRWL PDQFFVHPDY RDCHLLYYTG ITRTAKSILA EIVSSMFLNS GRHLSLLAEM |
| 841 | KAHAMDMSEA ILRSNFESFG RLVGKTWIQN QALDCGTNPP AVAAIIEKIK DYTLGYKLPG |
| 901 | AGGGGYLYMV AKDPQAAGQI RRILTEQAPN PRARFVEMTL SDKGLQVSRS |

In some embodiments, one or more modification are made to a microorganism (such as by genetic engineering) and/or to one or more nucleic acids encoding an enzyme for a step in making an HMO. Such modification can include, but are not limited to: a) replacement of an endogenous promoter with an exogenous promoter operably linked to the endogenous enzyme, such as gmd, gfs, fkp, and/or ft; b) expression of GMD, GFS, FKP and/or FT via an extrachromosomal genetic material; c) integration of one or more copies of gmd, gfs, fkp, and/or ft into the genome of the microorganism; or d) a modification to the endogenous gmd, gfs, fkp and/or ft to produce a modified gmd, gfs, fkp, and/or ft that encodes a protein that has an increased activity or any combination of modifications a) to d) described in this paragraph.

In some embodiments, an expression of GMD, GFS, and/or FT is varied by utilizing different promoters or changes immediately adjacent to the introduced gmd, gfs, fkp and/or ft genes. For example, in certain embodiments the deletion of a URA3 cassette adjacent to an introduced gmd, gfs, fkp, and/or ft expression cassette leads to a further improvement of 2′-FL production.

In some embodiments the endogenous promoter is replaced with an exogenous promoter that induces the expression at a higher level than the endogenous promoter. In certain embodiments, the exogenous promoter is specific for the microorganism in which the exogenous promoter replaces the endogenous promoter. For example, a yeast specific exogenous promoter can be used if the microorganism being modified is a yeast. The exogenous promoter can be a constitutive promoter or inducible promoter.

Nonlimiting examples of constitutive yeast specific promoters include: pCYC1, pADH1, pSTE5, pADH1, pCYC100 minimal, pCYC70 minimal, pCYC43 minimal, pCYC28 minimal, pCYC16, pPGK1, pCYC, pGPD or pTDH3. Additional examples of constitutive promoters from yeast and examples of constitutive promoters from microorganisms other than yeast are known to a skilled artisan and such embodiments are within the purview of the invention.

Non-limiting examples of inducible yeast specific promoters include: pGAL1, pMFA1, pMFA2, pSTE3, pURA3, pFIG1, pEVO2, pDLD, pJEN1, pmCYC, and pSTE2. Additional examples of inducible promoters from yeast and examples of inducible promoters from microorganisms other than yeast are known to a skilled artisan and such embodiments are within the purview of the invention.

Microorganisms used to produce the genetically modified microorganisms described herein may be selected from Saccharomyces spp., such as S. cerevisiae, pastorianus, S. beticus, S. fermentati, S. paradoxus, S. uvarum and S. bayanus; Schizosaceharomyces spp., such as S. pombe, S., japonicus, S. octosporus and S. cryophilus; Torulaspora spp. such as T. delbrueckii, Kluyveromyces spp. such as K. marxianus; Pichia spp. such as P. stipitis, P. pastoris or P. angusta, Zygosaccharomyces spp. such as Z. bailii; Brettanomyces spp. such as B. inter medius, B. bruxellensis, B. anomalus, B. custersianus, B. naardenensis, B. nanus; Dekkera spp., such as D. bruxellensis and D. anomala; Metschmkowia spp.; Issatchenkia spp. such as I. orientalis, Kloeckera spp., such as K. apiculata; Aureobasidium spp. such as A. pullulans; Torulaspora spp., Torulaspora delbrueckii, Zygosaccharomyces spp., Zygosaccharomyces bailii, Brettanomyces spp., Brettanomyces intermedius, Brettanomyces bruxellensis, Brettanomyces anomalus, Brettanomyces custersianus, Brettanomyces naardenensis, Brettanomyces nanus, Dekkera spp., Dekkera bruxellensis, Dekkera anomala, Metschmkowia spp., Issatchenkia spp., Issatchenkia orientalis, Issatchenkia terricola, Kloeckera spp., Kloeckera apiculate, Aureobasidium spp., Aureobasidium pullulans, Rhodotorula spp., Rhodotorula glutinis, Rhodotorula cladiensis, Rhodosporidium spp., Rhodosporidum toruloides, Cryptococcus spp., Cryptococcus neoformans, Cryptococcus albidus, Yarrowia spp., Yarrowia lipolytica, Kuraishia spp., Kuraishia capsulata, Kuraishia molischiana, Komagataella spp., Komagataella phaffii, Komagataella pastoris, Hanseniaspota spp., Hanseniaspora guilliermondii, Hanseniaspora uvarum, Hasegawaea spp., Hasegawaea japonica, Ascoidea spp., Ascoidea asiatica, Cephaloascus spp., Cephaloascus fragrans, Lipomyces spp., Lipomyces starkeyi, Kawasaki spp., Kawasakia arxii, Zygozyma spp., Zygozyma oligophaga, Metschnikowia spp., Metschnikowia pulcherrima, Coccidiodes spp., Coccidiodes immitis, Neurospora discreta, Neurospora africana, Aspergillus spp., Aspergillus nicer, Aspergillus nidulans, Aspergillus oryzae, Aspergillus fumigatus, Mucor spp., Mucor circinelloides, Mucor racemosus, Rhizopus spp., Rhizopus oryzae, Rhizopus stolonifera, Umbelopsis spp., Umbelapsis Mortierella spp., Mortierella alpine, Afternaria spp., Afternaria alternate, Bonytis spp., Botrytis cinereal, Fusarium spp., Fusarium graminarium, Geotrichum spp., Geotrichum candidum, spp., Penicillum chrysogenum, Chaetomium spp., Chaetomium thermophila, Magnaporthe spp., Magnaporthe grisea, Emericella spp., Emericella discophora, Trichoderma spp., Trichodema reesei, Talaromyces spp., Talaromyces emersonii, Sordaria spp., or Sordaria macrospora.

In specific embodiments, a microorganism, preferably, a fungus, such as a yeast, more preferably, a Saccharomyces spp., and even more preferably, S. cerevisiae is provided as die microorganism host. Yeast such as Saccharomyces spp. can be genetically engineered as described herein or using a multitude of available tools.

Other Ascomycetes fungi can also serve as suitable hosts. Many ascomycetes are useful industrial hosts for fermentation production. Exemplary genera include Trichoderma, Kluyveromyces, Yarrowia, Aspergillus, Schizosaccharomyces, Neurospora, Pichia (Hansenula) and Saccharomyces. Exemplary species include Pichia pastoris, Saccharomyces cerevisiae, Schizosaccharomyces pombe, Trichoderma reesei, Aspergillus niger, Aspergillus oryzae, Kluyveromyces lactis, Kluyveromyces marxianus, Neurospora crassa, Hansenula polymorpha, Yarrowia lipolytica, and Saccharomyces boulardii.

Cloning tools are widely known to those skilled in the art. See e.g., Cellulases and beyond: the first 70 years of the enzyme producer Trichoderma reesei, Robert H. Bischof, Microbial Cell Factories Volume 15, Article number: 106 (2016)), Development of a comprehensive set of tools for genome engineering in a cold- and thermo-tolerant Kluyveromyces marxianus yeast strain, Yumiko Nambu-Nishida, Scientific Reports volume 7, Article number: 8993 (2017); Engineering Kluyveromyces marxianus as a Robust Synthetic Biology Platform Host, Paul Cernak, mBio September 2018, 9 (5) e01410-18; DOI: 10.1128/mBio.01410-18; How a fungus shapes biotechnology: 100 years of Aspergillus niger research, Timothy C. Cairns, Fungal Biology and Biotechnology Volume 5, Article number: 13 (2018), GoldenPiCS: a Golden Gate-derived modular cloning system for applied synthetic biology in the yeast Pichia pastoris, Roland Prielhofer, BMC Systems Biology Volume 11, Article number: 123 (2017)), Aiko Ozaki, “Metabolic engineering of Schizosaccharomyces pombe via CRISPR-Cas9 genome editing for lactic acid production from glucose and cellobiose.” Metabolic Engineering Communications Volume 5, December 2017, Pages 60-67, World J Microbiol Biotechnol. 2019; 35(1): 10. “Yarrowia lipolytica: a beneficious yeast in biotechnology as a rare opportunistic fungal pathogen: a minireview,” Bartlomiej Zieniuk (2014) “Functional Heterologous Protein Expression by Genetically Engineered Probiotic Yeast Saccharomyces boulardii.” PLOS ONE 9(11).; “Metabolic Engineering of Probiotic Saccharomyces boulardii,” Liu J-J, Kong I I, 2016. Metabolic engineering of probiotic Saccharomyces boulardii. Appl Environ Microbiol 82:2280-2287; David Havlik, “Establishment of Neurospora crassa as a host for heterologous protein production using a human antibody fragment as a model product”, Microb Cell Fact. 2017; 16: 128; Ho, C. C. (April 1986), “Identity and characteristics of Neurospora intermedia responsible for oncom fermentation in Indonesia”. Food Microbiology. 3 (2): 115-432.

III. Enhancement of Production and Export of HMOs

In some embodiments, the production and/or export of an HMO can be enhanced through. genetic modification of an HMO-producing microorganism. For example, an HMO-producing microorganism can be modified by one or more of the following:

i) a genetic modification that increases the activity of PMA1 in the microorganism compared to PMA1 activity in the parental microorganism,
ii) a genetic modification that decreases the activity of SNF3 in the microorganism compared to SNF3 activity in the parental microorganism,
iii) a genetic modification that decreases the activity of RGT2 in the microorganism compared to RGT2 activity in the parental microorganism, and
iv) a genetic modification that decreases the activity of GPR1 in the microorganism compared to GPR1 activity in the parental microorganism.

In particular embodiments, i) the genetic modification that increases the activity of PMA1 is a genetic modification to plasma membrane ATPase gene (pma1), ii) the genetic modification that decreases the activity of SNF3 is a genetic modification to sucrose non-fermenting gene (snf3), iii) the genetic modification that decreases the activity of RGT2 is a genetic modification to glucose transport gene (rgt2), and iv) the genetic modification that decreases the activity of GPR1 is a genetic modification to G protein-coupled receptor 1 gene (gpr1). Examples of PMA1, SNF3, RGT2, and GPR1 are described in International Patent Application No. PCT/US2018/040351, the contents of which are incorporated herein by reference.

An example of PMA1 is provided by the sequence of SEQ ID NO: 5, which is PMA1. from Saccharomyces cerevisiae. Homologs of PMA1 from microorganisms other than S. cerevisiae, particularly, from yeast, can be used in the microorganisms and methods of the present disclosure. Non-limiting examples of the homologs of PMA1 useful in the instant disclosure are represented by Uniprot entries: A0A1U8I9G6, A0A1U8H4C1, A0A093V076, A0A1U8FCY1, Q08435, A0A1U7Y482, A0A1U8GLU7, P22180, A0A1U8G6C0, A0A1U8IAV5, A0A1U8FQ89, P09627, A0A199VNH3, P05030, P28877, A0A1U8I3U0, Q0EXL8, A0A1U813V7, P49380, Q07421, A0A1D8PJ01, P54211, P37367, P07038, Q0Q5F2, G8BGS3, A0A167F957, M5ENE2, A0A1B8GQT5, O74242, Q9GV97, Q6VAU4, A0A177AKN9, A0A1J6KB29, A0A2H9ZYJ6, A0A251U1M1, A0A251USM2, D2DVW3, M5BX73, Q6FXU5, A3LP36, G3ARI4, 9NSP9, A0A167C712, G2WE85, F2QNM0, A6ZUY5, C7GK65, A0A142GRJ4, W0T7K4, B3LDT4, A0A0H5BY16, A0A1B2J5T9, E7DB83, Q9UR20, F4NA03, Q96TH7, F4NA02, I2G7P2, C4PGL3, F4NA00, F4N9Z6, Q7Z8B7, F4N9Z9, A0A1L4AAP4, O94195, A0A1D1YKT6, A0A0U1YLR0, A0A0F8DBR8, A0A1C7N6N1, A0A2N6P2L5, A0A2C5WY03, O14437, T1VYW7, T1VYW7, A1KAB0, C0QE12, K0NAG7, A0A0H3J1I1, A0A1Q9D817, A0A068MZP7, D1JED6, A0A2K8WRE9, A0A1A8YFD7, A0A1A8YG89, I2G7P8, D9PN36, D1JI19, B6IUJ9, BIXP54, H8W7G4, H6SL18, G8LCW3, L8AJP6, Q5ZFR6, A0A1D7QSR3, A0A1Q2TYG8, F4N054, A0A1Q9CTB2, A0A1Q9EJV5, A0A1D1XEE3, A0A0F7GAE0, D2DVW4, A0A0A9YX23, A0A1Q9ELW6. The Uniprot entries listed herein are incorporated by reference in their entireties.

Additional homologs of PMA1 are known in the art and such embodiments are within the purview of the present disclosure. For example, the homologs of PMA1 have at least 60%, 65%, 70%, 75%, 80%, 85%, 90%, 95%, or 99% sequence identity to SEQ ID NO: 5.

| SEQ ID 5: | |
|---|---|
| 1 | mtdtssssss ssassvsahq ptqekpakty ddaasessdd ddidalieel qsnhgvdded |
| 61 | sdndgpvaag earpvpeeyl qtdpsyglts devlkrrkky glnqmadeke slvvkfvmff |
| 121 | vgpiqfvmea aailaaglsd wvdfgvicgl lmlnagvgfv qefqagsivd elkktlanta |
| 181 | vvirdgqlve ipanevvpgd ilqledgtvi ptdgrivted cflqidqsai tgeslavdkh |
| 241 | ygdqtfssst vkrgegfmvv tatgdntfvg raaalvnkaa ggqghftevl ngigiillvl |
| 301 | viatlllvwt acfyrtngiv rilrytlgit iigvpvglpa vvtttmavga aylakkgaiv |
| 361 | qklsaiesla gveilcsdkt gtltknklsl hepytvegvs pddlmltacl aasrkkkgld |
| 421 | aidkaflksl kqypkakdal tkykvlefhp fdpvskkvta vvespegeri vevkgaplfv |
| 481 | lktveedhpi pedvhenyen kvaelasrgf ralgvarkrg eghweilgvm pcmdpprddt |
| 541 | aqtvsearhl glrvkmltgd avgiaketcr qlglgtniyn aerlglgggg dmpgseladf |
| 601 | venadgfaev fpqhkyrvve ilqnrgylva mtgdgvndap slkkadtgia vegatdaars |
| 661 | aadivflapg lsaiidalkt srqifhrmys yvvyrialsl hleiflglwi aildnsldid |
| 721 | livfiaifad vatlaiaydn apyspkpvkw nlprlwgmsi ilgivlaigs witlttmflp |
| 781 | kggiignfga mngimflqis ltenwlifit raagpfwssi pswqlagavf avdiiatmft |
| 841 | lfgwwsenwt divtvvrvwi wsigifcvlg gfyyemstse afdrlmngkp mkekkstrsv |
| 901 | edfmaamqry stgheket |

An example of SNF3 is provided by the sequence of SEQ ID NO: 6, which is SNF3 from S. cerevisiae. Homologs of SNF3 from microorganisms other than S. cerevisiae, particularly, from yeast, can be used in the microorganisms and methods of the present disclosure. Non-limiting examples of the homologs of SNF3 useful in the instant disclosure are represented by Uniprot entries: W0TFH8, Q6FNU3, A0A0W0CEX1, G2WBX2, A6ZXD8, J6EGX9, P10870, C7GV56, B3LH76, A0A0L8RL87, A0A0K3C9L0, M7WSX8, A0A1U8HEQ5, G5EBN9, A8X3G5, A3LZS0, G3AQ67, A0A1E4RGT4, A0A1B2J9B3, F2QP27, E3MDL0, A0A2C5X045, G0NWE1, A0A0H5S3Z1, A0A2G5VCG9, A0A167ER19, A0A167DDU9, A0A167CY60, A0A167CEW8, A0A167ER43, A0A167F8X4, A0A1B8GC68, A0A177A9B0, E3EIS7, E3E8B6, A0A0A9Z0Q2. The Uniprot entries listed herein are incorporated by reference in their entireties.

Additional homologs of SNF3 are known in the art and such embodiments are within the purview of the present disclosure. For example, the homologs of SNF3 have at least 60%, 65%, 70% 75%, 80%, 85%, 90%, 95%, or 99% sequence identity to SEQ ID NO: 6.

| SEQ ID 6: | |
|---|---|
| 1 | mdpnsnssse tlrqekqgfl dkalqrvkgi alrrnnsnkd httddttgsi rtptslqrqn |
| 61 | sdrqsnmtsv ftddistidd nsilfseppq kqsmmmsicv gvfvavggfl fgydtglins |
| 121 | itsmnyvksh vapnhdsfta qqmsilvsfl slgtffgalt apfisdsygr kptiifstif |
| 181 | ifsignslqv gaggitlliv grvisgigig aisavvplyq aeathkslrg aiistyqwai |
| 241 | twgllvssav sqgtharnda ssyripiglq yvwssflaig mfflpespry yvlkdkldea |
| 301 | akslsflrgv pvhdsgllee lveikatydy easfgssnfi dcfissksrp kqtlrmftgi |
| 361 | alqafqqfsg infifyygvn ffnktgvsns ylvsfityav nvvfnvpglf fveffgrrkv |
| 421 | 1vvggvimti anfivaivgc slktvaaakv miaficlfia afsatwggvv wvisaelypl |
| 481 | gvrskctaic aaanwlvnfi calitpyivd tgshtssiga kiffiwgsln amgvivvylt |
| 541 | vyetkgltle eidelyikss tgvvspkfnk direralkfq ydplqrledg kntfvakrnn |
| 601 | fddetprndf rntisgeidh spnqkevhsi pervdiptst eilespnkss gmtvpvspsl |
| 661 | qdvpipqtte paeirtkyvd lgnglglnty nrgppslssd ssedytedei ggpssqgdqs |
| 721 | nrstmndind ymarlihsts tasnttdkfs gnqstlryht asshsdttee dsnlmdlgng |
| 781 | lalnaynrgp psilmnssde eanggetsdn lntaqdlagm kermaqfaqs yidkrgglep |
| 841 | etqsnilsts lsvmadtneh nneilhssee natnqpvnen ndlk |

An example of RGT2 is provided by the sequence of SEQ ID NO: 7, which is RGT2 from S. cerevisiae. Homologs of RGT2 from organisms other than S. cerevisiae, particularly, from yeast, can be used in the microorganisms and methods of the present disclosure. Non-limiting examples of the homologs of RGT2 are represented by Uniprot entries: A0A0U1MAJ7, N4TG48, A0A1 Q8RPY1, N4U7I0, A0A1L7SSQ2, A0A1L7VB15, A0A0C4E497, A0A1L7UAN6, A0A0J0CU17, A0A1L7VMA9, S0ED22, A0A1L7SD48, N1R8L8, A0A1L7V0N4, S3BYD3, E4UUU6, N4UPT5, N4U030, A0A0I9YK83, S0DJS4, A0A0U1LWH9, A0A0K6FSJ2, N1S6K7, A0A0J6F3E5, A0A1E4RS51, N4UTN2, A0A0G2E6D5, A0A1J9R914, A0A0F4GQX7, A0A1S9RLB9, A3M0N3, J9PF54, A0A074WC52, A0A0K6GI66, N1QHS4, G2WXK0, B2VVL4, B2WDK7, A0A1J9S6A1, G4N0E9, L7JEU7, L7INA5, A0A0L1HE99, A0A0J8QL36, A0A0H5CKW2, A0A0J6Y4E2, W0VMG0, G2WQD8, A0A1C1WV61, A0A1S9RL33, C9SBA9, A0A0G2HY75, J3P244, N1QK04, A0A0N0NQR9, A0A1S7UJ19, G2XFE7, C9SWZ3, R8BUY9, M7SYH1, A0A1E1MIV2, A0A1E1LLK3, A0A1E1LJE1, L7J4Y3, L7I304, A0A1L7XU29, A0A136JCY3, A0A0J8RG81, A0A177DW33, A0A1L7X792, W9C8U1, B2VXL1, A0A0L1HMG8, A0A178DQW4, A0A167V6F7, A0A166WR60, A0A162KLT6, A0A1L7X3D1, G3JQX8, Q7S9U8, E9F7A6, A0A1S7HPX9, A0A0G2G564, A0A0W0D0B3, A6ZXI9, Q12300, C7GKZ0, G2WC23, A0A0H5CAT9, J4U3Y8, A0A0L8RL54. The Uniprot entries listed herein are incorporated by reference in their entireties.

Additional homologs of RGT2 are known in the art and such embodiments are within the purview of the present disclosure. For example, the homologs of RGT2 have at least 60%, 65%, 70%, 75%, 80%, 85%, 90%, 95%, or 99% sequence identity to SEQ ID NO: 7.

| SEQ ID 7: | |
|---|---|
| 1 | mndsqnclrq reenshlnpg ndfghhqgae ctinhnnmph rnavtestnd teaksivmed |
| 61 | dpnayqisyt nnepagdgai ettsillsqp lplrsnvmsv lvgifyavgg flfgydtgli |
| 121 | nsitdmpyvk tyiapnhsyf ttsqiailvs flslgtffga liapyisdsy grkptimfst |
| 181 | avifsignsl qvasgglv11 ivgrvisgig igiisavvpl yqaeaaqknl rgaiissyqw |
| 241 | aitigllvss avsqgthskn gpssyripig lqyvwssila vgmiflpesp ryyvlkdeln |
| 301 | kaakslsflr glpiedpril eelveikaty dyeasfgpst lldcfktsen rpkqilrift |
| 361 | giaiqafqqa sginfifyyg vnffnntgvd nsylvsfisy avnvafsipg mylvdrigrr |
| 421 | pvllaggvim aianiviaiv gysegktvva skimiaficl fiaafsatwg gvvwvvsael |
| 481 | yplgvrskct aicaaanwlv nftcalitpy ivdvgshtss mgpkiffiwg glnvvavivv |
| 541 | yfavyetrgl tleeidelfr kapnsvissk wnkkirkrcl afpisqqiem ktniknagkl |
| 601 | dnnnspivqd dshriidvdg flenqiqsnd hmiaadkgsg slvniidtap ltstefkpve |
| 661 | hppvnyvdlg nglglntynr gppsiisdst defyeendss yynnnterng ansvntymaq |
| 721 | linsssttsn dtsfspshns nartssnwts dlaskhsqyt spq |

An example of GPR1 is provided by the sequence of SEQ ID NO: 8, which is GPR1 from S. cerevisiae. Homologs of GPR1 from microorganisms other than S. cerevisiae, particularly, from yeasts, can be used in the microorganisms and methods of the present disclosure. Non-limiting examples of the homologs of GPR1 are represented by Uniprot entries: A0A1S3ALF0, A0A0Q3MD25, A0A146RBQ8, A0A0P5SHA9, A2ARI4, Q9BXB1, Q9Z2H4, F1MLX5, U3DQD9, I2CVT9, I0FI44, K7D663, K7ASZ6, A0A1U7Q769, U3ESI5, T1E5B8, A0A0F7ZA01, J3RZW5, A0A094ZHC9, W6UL90, A0A0P6J7Q8, L5KYC3, B7P6N0, B0BLW3, A2AHQ2, A0A151N8W7, A0A146RCW3, A0A0X3NYB9, A0A0P5Y3G9, W5UAB2, A0A0P5IC44, A0A090XF51, A0A146NRV7, A0A0X3Q0R0, A0A0P6IRD7, L9JFB7, A0A146YGG2, A0A146WG88, Q12361, B3LGT6, A0A0N8A6F9, P0DM44, W6JM29, A0A1A8LC80, A0A0N8A4D4, Q7Z7M1, A0A1S3G1Q8, A0A1U7QGH1, A6ZXT8, A0A1U8C0F6, D3ZJU9, A0A1S3KGL3, G5B385, L9KNY9, A0A1S3AQM3, A0A087UXX9, A0A0L8VW24, A0A0P6AR08, Q9HBX8, Q3UVD5, A0A1U7UEF2, A0A146XMF9, A0A146QTV1, A0A1S31D45, L5KTU9, A0A1A8ELT4, A0A0N7ZMX8, A0A0P5Q3T8, A0A1A8N9Z4, A0A1A8D807, A0A1A8CVG1, A0A1A8UMB1, A0A1A8JQ07, A0A1A8P7N2, A0A1A8HL38, E7FE13, A0A1S3FZL3, A0A0P7WLQ9, H2KQN3, A0A1S3WJA9. A0A146PKA1, L5LLQ3, F1Q989, A0A0F8AKY3, A0A0P7VR95, A0A1U8C8I3, A0A034VIM3, A0A0N8BFD4, A0A146XMJ1, A0A0N8BDM1, A0A1A8KTJ1, A0A1A7X706, A0A0R4ITE3, A0A1U7S4H0, A0A1S3AQ94, A0A1U7UCP2, L8HMA8, A0A0Q3P3V6, A0A1A8CDG3, D6W7N2, A0A1E1XMY8, A0A1A8ACL5, A0A1S3WNV2, T0MHY5, A0A1S3G113, V8P2X5, A0A1S3KV51, A0A1S3G018, A0A1S3PUP5, A0A1U8C7X5, S9WP18, A0A1S3AQL8, A0A0N8ENF1, K7CIG0, A0A147BFY7, A0A1S3FZK9, A0A1U7TUH0, A0A1U8BX93, A0A091DKN5, A0A146W919, A0A147B2K7, A0A146XNL4, A0A091DTX9, A0A0Q3UQB0, A0A146WH37, E9QDD1, Q58Y75, A0A096MKI0, A0A1S3S901, Q14BH6, A0A1S3AQ42, A0A0P5SV49, A0A0P5P299, A0A0P5WCR4, K7CHT8, A0A1U7U0Q5, A0A1S3EXD4, A0A146Y6G0, A0A061HX0, A0A1S3AQ84, A0A1S2ZNQ3, A0A1U7UEE6, A0A1S3G013, A0A1U7QJG4, S7N7M1, A0A1S3G108, A0A1U8C8H8, and A0A1U8C7X0.

Additional homologs of GPR1 are known in the art and such embodiments are within the purview of the present disclosure. For example, the homologs of GPR1 have at least 60%, 65%, 70%, 75%, 80%, 85%, 90%, 95%, or 99% sequence identity to SEQ ID NO: 8.

| SEQ ID 8: | |
|---|---|
| 1 | mitegfppnl nalkgsslle krvdslrqln tttvnqllgl pgmtstftap qllqlriiai |
| 61 | tasavsliag clgmfflskm dkrrkvfrhd liafliicdf lkafilmiyp miilinnsvy |
| 121 | atpaffntlg wftafaiega dmaimifaih failifkpnw kwrnkrsgnm egglykkrsy |
| 181 | iwpitalvpa ilaslafiny nklnddsdtt iildnnnynf pdsprqggyk pwsawcylpp |
| 241 | kpywykivls wgpryfiiif ifavylsiyi fitseskrik aqigdfnhnv leeekekkkl |
| 301 | fglghwgkak wyfrsyfklp llhllrnlkn fftisfidpn eetddsgssn gtfnfgessn |
| 361 | eiptlfrktn tgsdenvsas ggvrlldyns akpldmskya mseqpdlern npfdcendit |
| 421 | lnpselvskq kehkvtfsve negldtrkss mlghqtfscq nslesplamy dnkndnsdit |
| 481 | snikekggii nnnsnndddd nnnnndndnd nnnsnnnnnn nnnnnnnnnn nnnnnnnnnn |
| 541 | nnnnsnnikn nvdnnntnpa dniptlsnea ftpsqqfsqe rvnnnadrce nssftnvqqh |
| 601 | fqaqtykqmk krraqiqknl raifiyplsy igiwlfpiia dalqynheik hgptmwvtyi |
| 661 | dtcvrplscl vdvivylfke kpwnyswakt eskyliekyi lkgelgekei lkfchsnwgk |
| 721 | rgwyyrgkwk krkcwkystn plkrilwfve rffkqlfelk lhfsfydncd dfeywenyys |
| 781 | akdsndnkrt esdetktnss drslpsnsle lqaminnita eevevplfwr iihhipmlgg |
| 841 | idldelnrll kirynndhfs lpglkfalnq nkshdkhqdv stnsmvkssf fssnivtndd |
| 901 | ensieedknl rysdasasen ylvkptipgt tpdpiieaqn dndssdssgi dliaflrngp |
| 961 | l |

Substrates for Production of HMOs

In certain embodiments, the present disclosure provides microorganisms comprising one or more genetic modifications that provide for import and/or enhanced uptake of one or more substrates that can be used by the microorganism to make an HMO. For example, a microorganism can include:
i) a genetic modification that introduces a substrate transporter gene LAC12, or its analogues which increases the uptake of lactose and/or other substrate into the microorganism;
ii) a genetic modification that introduces a transporter which can both import a substrate, such as lactose and export a produced HMO, such as the wild type cellodextrin transporter gene cdt-1 or a variant of the cellodextrin transporter gene cdt-1 such as those described herein (for example, CDT-1 N209S F262Y, CDT-1 G91A, CDT-1 F213A, CDT-1 L256V, CDT-1. F335A, CDT-1 S411A, CDT-1 N209S F262W).

| Lactose transporter (Lac12) [Kluyveromyces lactis] | |
|---|---|
| SEQ ID NO: 41 | |
| 1 | MADHSSSSSS LQKKPINTIE HKDTLGNDRD HKEALNSDND NTSGLKINGV PIEDAREEVL |
| 61 | LPGYLSKQYY KLYGLCFITY LCATMQGYDG ALMGSIYTED AYLKYYHLDI NSSSGTGLVF |
| 121 | SIFNVGQICG AFFVPLMDWK GRKPAILIGC LGVVIGAIIS SLTTTKSALI GGRWFVAFFA |
| 181 | TIANAAAPTY CAEVAPAHLR GKVAGLYNTL WSVGSIVAAF STYGTNKNFP NSSKAFKIPL |
| 241 | YLQMMFPGLV CIFGWLIPES PRWLVGVGRE EEAREFIIKY HLNGDRTHPL LDMEMAEIIE |
| 301 | SFHGTDLSNP LEMLDVRSLF RTRSDRYRAM LVILMAWTGQ FSGNNVCSYY LPTMLRNVGM |
| 361 | KSVSLNVLMN GVYSIVTWIS SICGAFFIDK IGRREGFLGS ISGAALALTG LSICTARYEK |
| 421 | TKKKSASNGA LVFIYLFGGI FSFAFTPMQS MYSTEVSTNL TRSKAQLLNF VVSGVAQFVN |
| 481 | QFATPKAMKN IKYWFYVFYV FFDIFEFIVI YFFFVETKGR SLEELEVVFE APNPRKASVD |
| 541 | QAFLAQVRAT LVQRNDVRVA NAQNLKEQEP LKSDADHVEK LSEAESV |

Production, Separation and Isolation of HMOs

In some embodiments, the microorganisms described herein are capable of producing HMOs such as 2′-FL. In some embodiments, the microorganisms are capable of converting lactose into 2′-FL. In particular embodiments, the microorganisms described herein have higher capacity, compared to the parental microorganisms, of converting lactose into 2′-FL. In specific embodiments, the conversion of lactose into 2′-FL occurs in the cytosol of the microorganisms.

In still another aspect, methods of producing products of interest by culturing the microorganisms described herein in appropriate media containing an appropriate oligosaccharide under appropriate conditions for an appropriate period of time and recovering an oligosaccharide from the culture media, is provided.

In certain embodiments, the disclosure provides methods of producing 2′-FL by culturing the microorganisms described herein in culture media containing lactose under appropriate conditions for an appropriate period of time and recovering 2′-FL from the culture media.

In preferred embodiments, the microorganisms belong to Saccharomyces spp. In even more preferred embodiments, the microorganisms are S. cerevisiae.

In certain embodiments, the media contains about 10 g/L yeast extract, 20 g/L peptone, and about 40 g/L oligosaccharide, particularly, lactose or sucrose. In particular embodiments, the microorganisms, particularly, yeast, are grown at 30° C.

Additional culture media, conditions appropriate for culturing the microorganisms, and the methods of recovering the products of interest from the culture media are well known in the art and such embodiments are within the purview of the invention.

In certain aspects, the present disclosure provides methods for producing oligosaccharides by culturing the microorganisms described herein in the presence of appropriate oligosaccharides and recovering the products of interest. In some embodiments, an HMO is separated from the cells (microorganism) that produce the HMO. In some cases, an HMO can be further isolated from other constituents of the culture media (fermentation broth) in which the HMO-producing cells are grown. in some embodiment, an HMO is recovered from the fermentation broth (also referred to a culture medium). Many methods are available for separation of cells and/or cell debris and other broth constituents from the produced HMO.

For example, cell/debris separation can be achieved through centrifugation and/or filtration. The filtration can be microfiltration or ultrafiltration or a combination thereof. Separation of charged compounds can be achieved through ion exchange chromatography, nanofiltration, electrodialysis or combinations thereof. Ion exchange chromatography can be cation or anion exchange chromatography, and can be performed in normal mode or as simulated moving bed (SMB) chromatography. Other types of chromatography may be used to separate based upon size (size exclusion chromatography) or affinity towards a specific target molecule (affinity chromatography). For example, US 2019/0119314 A1, GRAS applications GRN0005718 and GRN 000749.

Drying or concentration steps can be achieved with evaporation, lyophilization, reverse osmosis, or spray drying. Crystallization can serve as a concentration and separation step and can be done with for example evaporative or temperature-based crystallization, or induced by modification of pH or increase in ionic strength. For example, US20170369920A1, WO2018164937 A1.

Absorption techniques, such as adsorption using activated charcoal, can also be used as a separation step and in particular is useful for removal of color bodies or separation of oligosaccharides from monomers.

An HMO product can also be pasteurized, filtered, or otherwise sterilized for food quality purposes.

### Products and Compositions

The microorganisms and methods described herein can be used to produce a variety of products and compositions containing one or more HMOs. In some embodiments, a product suitable for animal consumption includes one or more HMO produced by the microorganisms or methods herein. The product can include one or more additional consumable ingredients, such as a protein, a lipid, a vitamin, a mineral or any combination thereof. The product can be suitable for mammalian consumption, human consumption or consumption as an animal feed or supplement for livestock and companion animals. In some embodiments, the product is suitable for mammalian. consumption, such as for human consumption and is an infant formula, an infant food, a nutritional supplement or a prebiotic product. Products can have 1, 2, 3 or more than 3 HMOs, and one or more of the HMOs can be produced by the microorganisms or by the methods described herein. In some cases, the HMO is 2′-fucosyllactose (2′-FL), 3′-fucosyllactose (3′-FL), 3′-sialyllactose (3′-SL), 6′-sialyllactose (6′-SL), lacto-N-neotetraose (LNnT), lacto-N-tetraose (LNT), sialyllacto-N-tetraose a (LST a), sialyllacto-N-neotetraose c (LST c), lacto-difucotetraose (LDFT) or lacto-N-fucopentaose I (LNFP I), or any combinations thereof.

### Exemplary Embodiments

In some embodiment, an engineered microorganism for production of an HMO comprises one or more of the following genetic modifications.
a) a genetic modification producing a GFS enzyme;
b) a genetic modification producing a GMD enzyme;
c) a genetic modification producing a FT enzyme;
d) genetic modifications producing any combination of GFS, GMD and FT enzymes;
e) a genetic modification producing a transporter for export of an HMO, for example CDT-1 or a variant of CDT-1 such as one of CDT-1 N209S F262Y, CDT-1 G91A, CDT-1 F213A, CDT-1 L256V, CDT-1 F335A, CDT-1 S411A, CDT-1 N209S F262W, one or more amino acid changes that correspond to one or more of positions predicted to be near the sugar substrate binding pocket and/or the PESPR motif such as positions G336, Q337, N341, and G471;
f) genetic modifications producing any combination of GMD, GFS and FT enzymes and a transporter for export of an HMO, for example CDT-1 or a variant of CDT-1 such as one of CDT-1 N209S F262Y, CDT-1 G91A, CDT-1 F213A, CDT-1 L256V, CDT-1 F335A, CDT-1 S411A, CDT-1 N209S F262W;
g) a genetic modification of any of the embodiments (a)-(f) and the CDT-1 can have one or more amino acid changes that correspond to one or more of positions predicted to be near the sugar substrate binding pocket the PESPR motif such as positions G336, Q337, N341, and G471.
h) a genetic modification producing a transporter for import of a substrate, such as lactose, for production of an HMO, for example Lac12, CDT-1 or a variant or analog thereof;
i) genetic modifications producing any combination of GMD, GFS and FT enzymes and a transporter for import of a substrate, such as lactose, for production of an HMO, for example Lac12, CDT-1 or a variant or analog thereof;
j) genetic modifications producing any combination of GMD, GFS and FT enzymes, a transporter for import of a substrate, such as lactose, for production of an HMO, for example Lac12, CDT-1 or a variant or analog thereof, and a transporter for export of an HMO, for example CDT-1 or a variant of CDT-1 such as one of CDT-1 N209S F262Y, CDT-1 G91A, CDT-1 F213A, CDT-1 L256V, CDT-1 F335A, CDT-1 S411A, CDT-1 N209S F262W;
k) supplying a substrate to a genetically modified microorganism for production of an HMO, such as lactose, and one or more of the modifications of a)-j);
l) production of an HMO in a genetically engineered microorganism, wherein the HMO is a non-branched HMO comprised of a lactose core, such as 2′-fucosyllactose (2′-FL), 3′-fucosyllactose (3′-FL), 3′-sialyllactose (3′-SL), 6′-sialyllactose (6′-SL), lacto-N-neotetraose (LNnT), lacto-N-tetraose (LNT), sialyllacto-N-tetraose (LST a), sialyllacto-N-neotetraose c (LST c), lacto-difucotetraose (LDFT) or lacto-N-fucopentaose I (LNFP I).
m) any of a)-l) wherein the microorganism is a Ascomycetes fungus, including but not limited to, a Saccharomyces spp., a Schizosaccharomyces spp., a Pichia spp., Trichoderma, Kluyveromyces, Yarrowia, Aspergillus, and Neurospora.

### EXAMPLES

### Example 1: Improved 2′-FL Production in Saccharomyces cerevisiae Expressing GMD, GFS, and/or FT

Expression vectors conferring well known activities for the enzymes GMD, GFS and FT (named GMD_t, GFS_t and FT_t) were generated for expression in the yeast Saccharomyces cerevisiae. Under selection pressure, these expression vectors are believed to occur in multiple tens of copies per cell and thus expression of a plasmid born gene is likely higher than from a single genomic locus if comparable promoters are used.

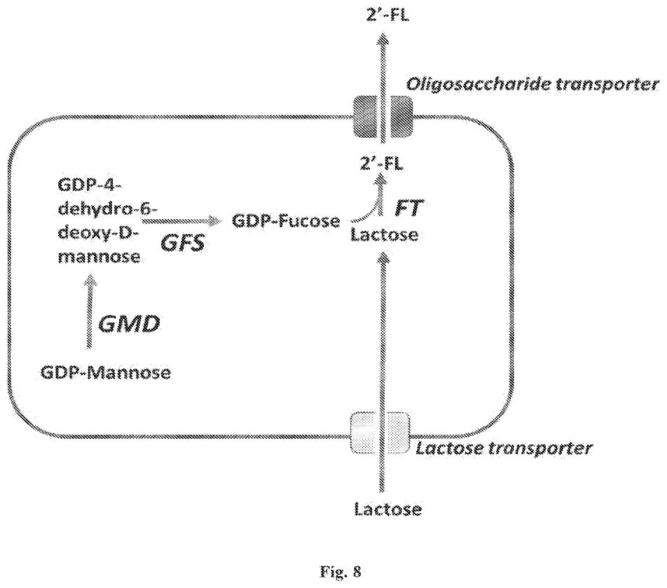

Constructs expressing heterologous GMD, GFS or FT genes were then co-transformed with plasmids containing all but the genes for which enzymatic activity was to be tested. The acceptor strain was a genetically modified Saccharomyces cerevisiae strain producing low titers of 2′-FL if grown on lactose. The strain also expresses Lac12 from Kluyveromyces lactis for improved import of lactose and an engineered oligosaccharide transporter for improved export of 2′-FL as indicated in FIG. 8.

After introduction of the plasmids GMD_t, GFS_t and FT_t, higher levels of 2′-FL were produced. The base strain was auxotrophic for the synthesis of Leucine, Histidine and Uracil while plasmids carried individual gene cassettes restoring auxotrophy for the respective compounds, respectively.

Omitting one of the plasmids restored 2′-FL production rates similar to the acceptor strain and, vice versa, additional expression of a gene encoding a protein that can functionally compensate for the lack of such enzymatic activity will increase 2′-FL production.

Putative GFSs were tested by transforming an expression construct comprising the putative GFS gene together with expression constructs containing GNMD_t and FT_t. After transformation, cells were selected on respective media omitting the compound for which transformed plasmids conferred auxotrophy for.

Colonies forming after the transformation were grown in drop out medium (omitting the compound the transformed plasmids conferred auxotrophy for) overnight at 30° C. and 250 rpm shaking. Cells were then washed and then transferred into YP4D0.4L medium, which is YPD medium with 0.4 g/L lactose and 4 g/L Glucose, and grown for 6 days under identical conditions. Supernatants were analyzed by HPLC analysis.

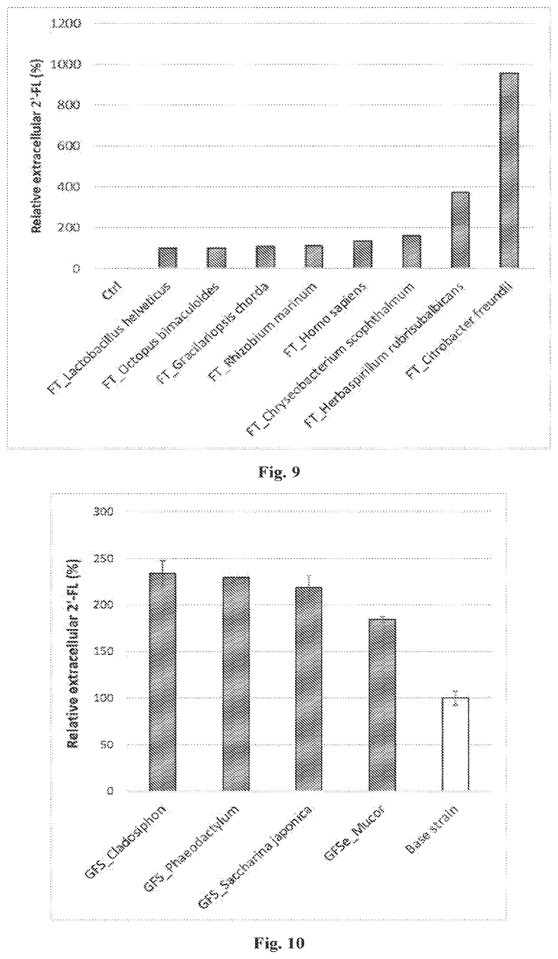

FIG. 9 shows 2′-FL production by introducing a heterologous fucosyltransferase (FT) from different organisms to a yeast strain which also expresses CDT-1 M7, GMD and WcaG from plasmids. Ctrl is control strain without FT expression.

FIG. 10 shows 2′-FL formation compared to the base strain, which was capable of producing lower amounts of 2′-FL with integrated 2′-FL pathway consist of GMD, WcaG and WbgL. Strains expressing plasmids with a GMD, a FT and a plasmid expressing a GFS selected from SEQ ID NOs: 20, 21, 22, and 23, respectively, produced significantly more 2′-FL than the base strain.

Likewise, putative FTs were tested by preparing expression constructs containing GMD_t and GFS_t. An additional plasmid carrying each one of the Fucose transferase genes from SEQ ID NO: 38, 29, 30, 31, 32, and 40 was included in each of these transformations. Cells were transformed with expression plasmids GMD_t, GFS_t and expression plasmids carrying each one of the FTs genes from SEQ ID NO: 38, 29, 30, 31, 32, and 40 and then selected, grown an analyzed as indicated above.

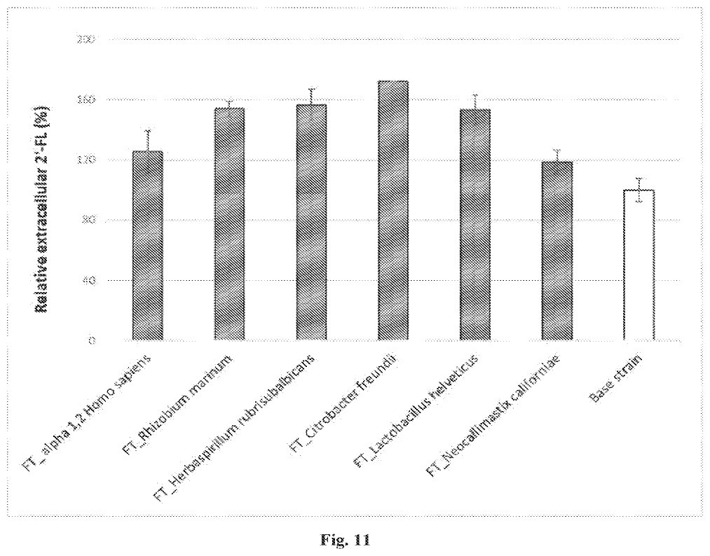

FIG. 11 shows that strains expressing various FTs accumulate more 2′-FL compared to the base strain.

The activity of an enzyme represented by SEQ ID NO: 24 was tested. This enzyme consists of 2 modules, one that has homology to GDP-Mannose-Dehydratases and one that shares homology with GDP fucose synthases. An enzyme comprising both, GMD and GFS, activities would hence be able to produce GDP fucose from GDP Mannose, NADPH⁺H⁺ and GTP.

A base strain capable of low level 2′-FL biosynthesis as described above was transformed with plasmids expressing i) a GMD, a FT and SEQ ID NO: 24 and ii) a FT and SEQ ID NO: 24 only. Cells were transformed, selected and grown as described above. Compared to the base strain, both combinations yielded higher 2′-FL production when compared to the base strain without expression of additional plasmids. The addition of plasmids expressing SEQ ID NO: 24 in absence of an additional plasmids expressing a fucose synthase significantly increases 2′-FL production compared to the base strain. Expression of a plasmid carrying a GMD gene in addition to plasmids carrying a FT and SEQ ID NO: 24 further 2′-FL production.

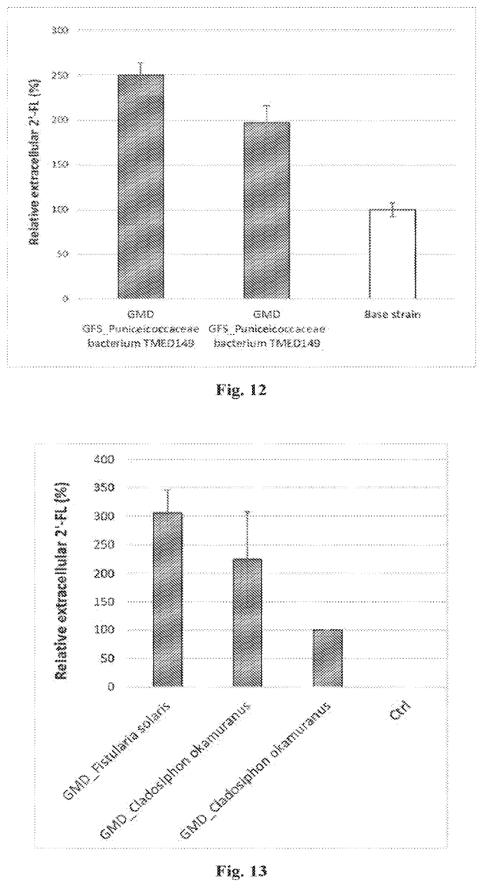

FIG. 12 shows relative production of 2′-FL in yeast cells expressing plasmids with (1st column) GMD, a FT and SEQ ID NO: 24 and (2nd column) plasmids with a FT and SEQ ID NO: 24 only, relative to a base strain that contains a set of genomic. GMD, GFS and FT genes.

### Fermentation and Metabolite Analysis

Triplicates of single colonies were inoculated in 10 mL of YPD and incubated at 30° C. overnight. The final fermentation volume was 10 mL in YPDL medium. The cells were incubated at 30° C. and 250 rpm for 120h. Lactose concentration was determined by high performance liquid chromatography on a Prominence HPLC (Shimazu, Kyoto, Japan) equipped with Rezex ROA-Organic Acid H 10×7.8 mm column. The column was eluted with 0.005 N of sulfuric acid at a flow rate of 0.6 mL/min, 50° C. 2′-FL concentration as determined using an ICS-3000 Ion Chromatography System (Dionex, Sunnyvale, Calif., USA) equipped with CarboPac PA20 column. The column was eluted with KOH gradient at a flow rate of 0.4 mL/min, 30° C.

### Example 2: 2′-FL Production in Saccharomyces cerevisiae, which Lacks 2′-FL Biosynthesis, by Expressing GMD, GFS, and/or FT

A base strain only carrying Lac12 for improved lactose import and an engineered membrane transporter for improved 2′-FL export as indicated in FIG. 8 was prepared. However, while this strain lacks any genes for 2′-FL biosynthesis it also had not been improved for 2′-FL biosynthesis. This base strain was transformed with plasmids expressing the GMDs encoded by SEQ ID NOs i) 17, ii) 18, and iii) 19. 2′-FL was produced in all these strains indicating that GMDs encoded by SEQ ID NOs: 17, 18, and 19, respectively all confer GMD activity if expressed in yeast cells.

FIG. 13 shows production of 2′-FL by expression of plasmids in a control strain otherwise not capable of 2′-FL production (Ctrl). Strains were transformed with plasmids expressing a GFS and a FT along with a plasmid carrying either SEQ ID NO: 17, 18, or 19, respectively. The control strain carrying no plasmids does not produce any 2′-FL.

### Example 3: Increase in 2′-FL Production in Saccharomyces cerevisiae Expressing CDT-1 N209S/F262Y

### Strains and Media

S. cerevisiae was grown and maintained on YPD medium (10 g/L yeast extract, 20 g/L peptone, 20 g/L glucose) at 30° C. All genes were expressed chromosomally. The cdt-1sy gene (encoding CDT-1 N209S/F262Y) was expressed within a background strain producing 2′-FL and 2′-FL accumulation in the growth medium was during a fermentation experiment was compared to the 2′-FL accumulation produced from the same strain without the cdt-1-sy gene.

The 2′-FL producing utilizing strain contains GDP-mannose-4,6-dehydratase (gmd1), GDP-L-fucose synthase (wca/G), lactose permease (LAC12) and two fucosyltransferases (FucT2, wbgL).

The experiments were conducted in YR medium (10 g/L yeast extract, 20 g/L peptone, 30 g/L glucose 2 g/L lactose) at 30° C.

### Fermentation and Metabolite Analysis

Triplicates of single colonies were inoculated in 10 mL of YPD and incubated at 30° C. overnight. The final fermentation volume was 10 mL in YPDL medium. The cells were incubated at 30° C. and 250 rpm for 1201h. Lactose concentration was determined by high performance liquid chromatography on a Prominence HPLC (Shimazu, Kyoto, Japan) equipped with Rezex ROA-Organic Acid H 10×7.8 mm column. The column was eluted with 0.005 N of sulfuric acid at a flow rate of 0.6 mL/min, 50° C. 2′-FL concentration as determined using an ICS-3000 Ion Chromatography System (Dionex, Sunnyvale, Calif., USA) equipped with CarboPac PA20 column. The column was eluted with KOH gradient at a flow rate of 0.4 mL/min, 30° C.

The cdt-1sy, gene (encoding CDT-1 N2095/F262Y) was expressed within a background strain producing 2′-FL and 2′-FL accumulation in the growth medium was during a fermentation experiment was compared to the 2′-FL accumulation produced from the same strain without the cdt-1-sy gene.

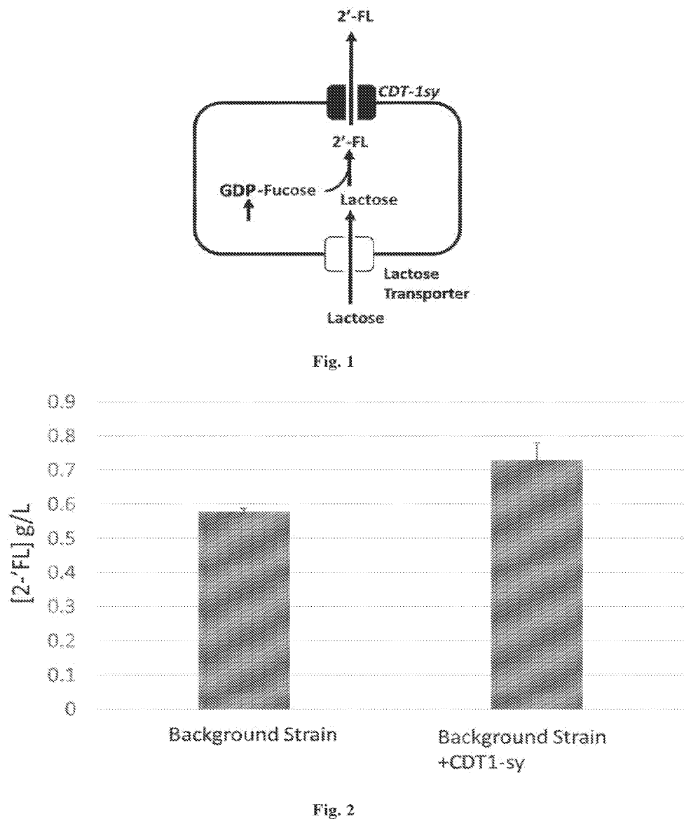

Unexpectedly, the expression of CDT-1 N209S/F262Y significantly increased the accumulation of 2′-FL within the growth medium (FIG. 2), indicating that CDT-1St can act as an efficient substrate exporter.

### Example 4: Increase in 2′-FL Production in Saccharomyces cerevisiae Expressing CDT-1 Mutants

### Strains and Media

The 2′-FL producing S. cerevisiae strain contains genome integrated Lad 2 or CDT-1 mutants as transporter and 2′-FL, producing pathway on pRS424, and pRS426 plasmids consist of GDP-mannose-4,6-dehydratase (gmd1), GDP-4-keto-6-deoxy-D-mannose 3,5-epimerase-4-reductase (wcaG), and fucosyltransferases (wkgL).

S. cerevisiae was initially grown and maintained in YPD medium (10 g/L yeast extract, 20 g/L peptone, 20 gi′L glucose) at 30° C. Optimized minimum medium (oMM) (See Lin Y. et al., Biotechnol Biofuels. 2014 Aug. 27; 7(1):126) with 2.0 g/L of glucose was used for preculture of yeast cells. Verdyun medium (See Verduyn et al., Yeast. 1992 July; 8(7):501-17, see the World Wide Web at apz-rl.de/002_download/003_mitgeltende_dokumente/012_Verduyn-Medium_002.pdf) with 60 g/L glucose and 6 g/L lactose (V60D6L) was used for 2′-FL production.

### Lactose Uptake Test

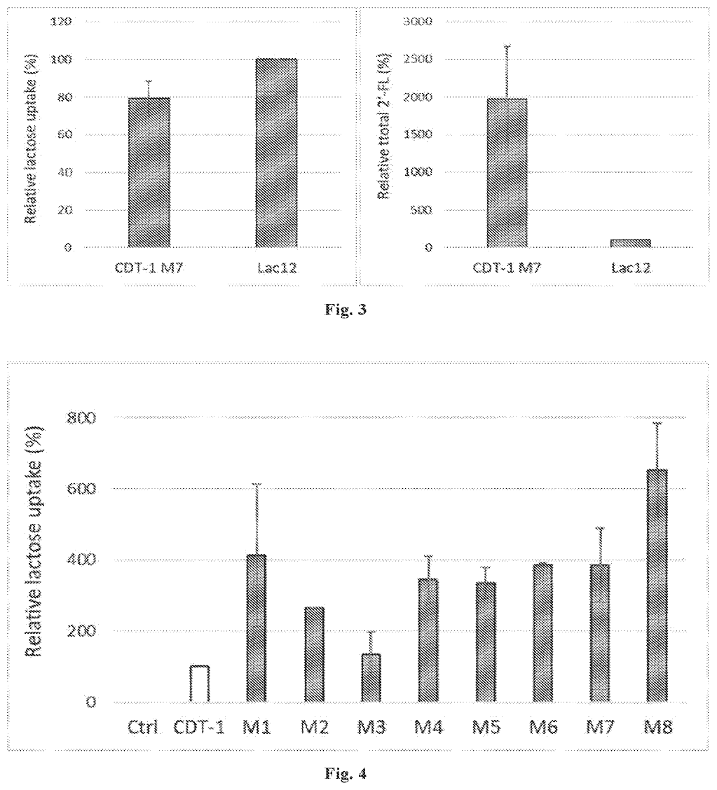

To measure lactose uptake, yeast strains with different transporters were grown in 4 mL YPD medium overnight at 30° C. and 250 rpm. Wild type yeast strain without transporter was used as control. The cell density was measured by a plate reader and was converted to Dry Cell Weight (DCW) The cell culture was washed in water and resuspended in lactose solution. The supernatant was analyzed by HPLC and lactose uptake was normalized by DCW. The lactose uptake from strains expressing CDT-1 mutant was normalized by lactose uptake from strain expressing wild type CDT-1 and shown as relative values in FIGS. 3 and 4.

### Fermentation and Metabolite Analysis

Triplicates of single colonies were inoculated in 10 mL of oMM medium with 20 g/L glucose and incubated at 30° C. overnight. The cell cultures were centrifuged and resuspended in 10 mL V60D6L medium and incubated at 30° C. and 250 rpm for 48 hours. Extracellular lactose, glucose, and 2′-FL concentration was determined by high performance liquid chromatography (HPLC) equipped with Rezex ROA-Organic Acid H 10×7.8 mm column and a refractive index detector (RID). The column was eluted with 0.005 N of sulfuric acid at a flow rate of 0.6 mL/min, 50° C. To measure total (intracellular and extracellular) 2′-FL, the fermentation broth containing yeast cells was boiled to release all of the intracellular 2-′FL. The supernatant was then analyzed by HPLC.

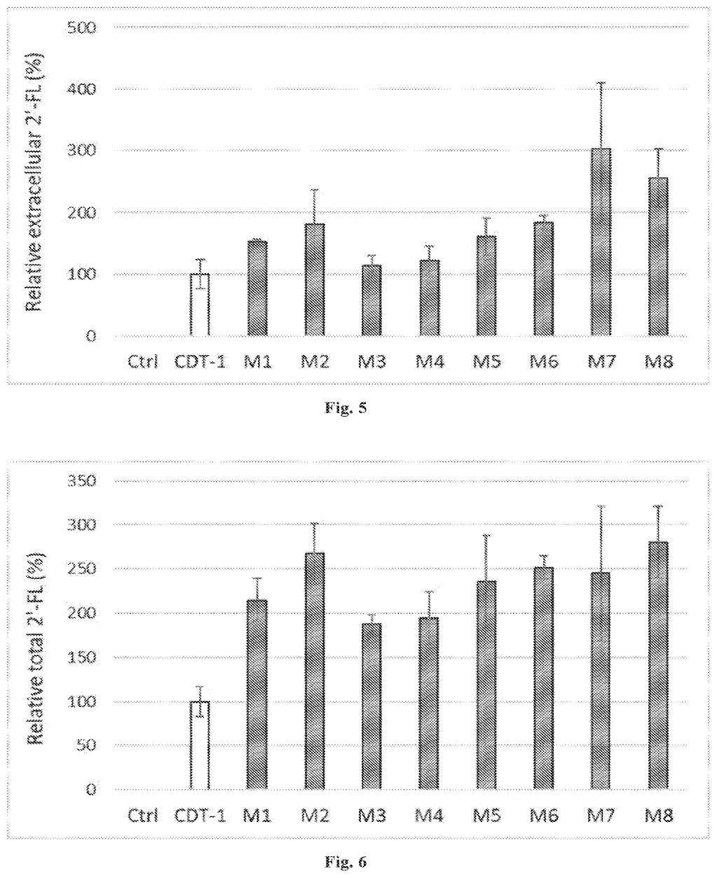

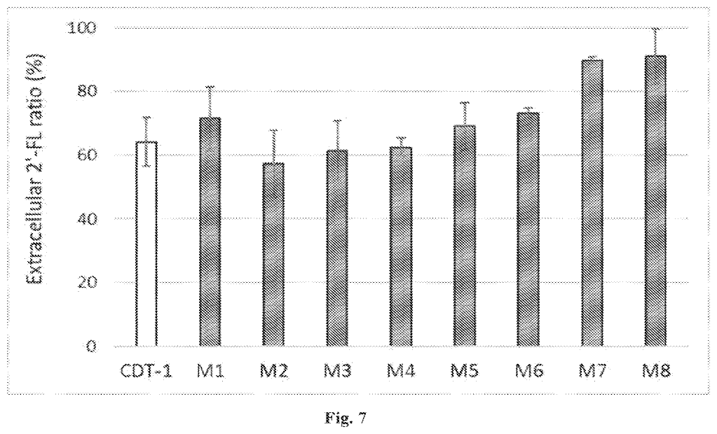

The extracellular and total 2′-FL titer shown in percentage in FIGS. 5-7 was normalized by the titer of strains with wild type CDT-1 Extracellular 2′-FL ratio (%) was calculated as follows: (extracellular 2′-FL-titer)/(total 2′-FL titer)×1.00%.

### INCORPORATION BY REFERENCE

Each of the patents, published patent applications, and non-patent references cited herein are hereby incorporated by reference in their entirety.

### EQUIVALENTS

Those skilled in the art will recognize, or be able to ascertain using no more than routine experimentation, many equivalents to the specific embodiments described herein. Such equivalents are intended to be encompassed by the following claims.

1. A microorganism for enhanced production of a human milk oligosaccharide (HMO) comprising a heterologous CDT-1 transporter variant and at least one heterologous pathway gene for production of the HMO, wherein the microorganism is capable of producing and exporting the HMO.

2-4. (canceled)

5. The microorganism according to claim 1, wherein the CDT-1 transporter variant has an amino sequence of SEQ ID NO:4 or a sequence with at least 80% homology thereto.

6. (canceled)

7. The microorganism according to claim 1, wherein the transporter comprises a sequence having one or more amino acid replacements at positions corresponding to amino acid positions 91, 209, 256, 262, 335, 411 of SEQ ID NO:4.

8. The microorganism according to claim 1, wherein the CDT-1 variant is encoded by a codon optimized nucleic acid.

9. (canceled)

10. The microorganism according to claim 5, wherein the transporter comprises an amino acid replacement selected from the group consisting of 91A, 209S, 256V, 262Y, 262W, 335A, 411A and any combination thereof.

11. The microorganism according to claim 1, wherein the pathway gene is selected from a GDP-mannose 4,6-dehydratase, a GDP-L-fucose synthase, and an α-1,2-fucosyl transferase.

12. (canceled)

13. The microorganism according to claim 1, wherein the HMO is selected from the group consisting of 2′-fucosyllactose (2′-FL), 3′-fucosyllactose (3′-FL), 3′-sialyllactose (3′-SL), 6′-sialyllactose (6′-SL), lacto-N-neotetraose (LNnT), lacto-N-tetraose (LNT), sialyllacto-N-tetraose a (LST a), sialyllacto-N-neotetraose c (LST c), lacto-difucotetraose (LDFT) and lacto-N-fucopentaose I (LNFP I).

14. The microorganism of claim 13, wherein the HMO is 2′-fucosyllactose.

15. The microorganism according to claim 1, wherein the microorganism is an Ascomycetes fungus.

16. The microorganism of claim 15, wherein the Ascomycetes fungus is selected from the group consisting of a Saccharomyces spp., a Schizosaccharomyces spp. and a Pichia spp.

17-20. (canceled)

21. The microorganism of claim 14, comprising a set of pathway genes for production of the HMO and the set comprises GDP-mannose 4,6-dehydratase (GMD), a GDP-L-fucose synthase (GFS), and a fucosyl transferase (FT).

22-25. (canceled)

26. The microorganism of claim 21, where the set of pathway genes comprises Gmd, WcaG and WbgL.

27. The microorganism of claim 21, wherein the GDP-mannose 4,6-dehydratase is selected from SEQ ID Nos. 17-19, 42, and 61-63 or a variant having at least 85% homology thereto.

28. The microorganism of claim 21, wherein the GDP-L-fucose synthase is selected from SEQ ID Nos. 20-23 or a variant having at least 85% homology thereto.

29. The microorganism of claim 21, wherein the α-1,2-fucosyl transferase is selected from SEQ ID Nos. 26-40 or a variant having at least 85% homology thereto.

30. A method of producing an HMO comprising:
providing a culture medium with at least one carbon source;
providing a microorganism of claim 1; and
culturing microorganism in the culture medium;
wherein a substantial portion of the HMO is exported into the culture medium.

31-43. (canceled)

44. The method according to claim 30, wherein the HMO is selected from the group consisting of 2′-fucosyllactose (2′-FL), 3′-fucosyllactose (3′-FL), 3′-sialyllactose (3′-SL), 6′-sialyllactose (6′-SL), lacto-N-neotetraose (LNnT), lacto-N-tetraose (LNT), sialyllacto-N-tetraose a (LST a), sialyllacto-N-neotetraose c (LST c), lacto-difucotetraose (LDFT) and lacto-N-fucopentaose I (LNFP I).

45-52. (canceled)

53. The microorganism of claim 1, further comprising a genetic modification that decreases the activity of SNF3 or RGT2 as compared to a parental microorganism without the genetic modification.

54. The microorganism of claim 1, wherein the microorganism is capable of exporting an increased amount of HMO as compared to a microorganism comprising a wild type heterologous CDT-1 transporter and the at least one heterologous pathway gene.

55. A microorganism for enhanced production of 2′-FL comprising a heterologous CDT-1 transporter or a variant thereof and at least one heterologous pathway gene for production of the HMO selected from the group consisting of GDP-mannose 4,6-dehydratase (GMD), a GDP-L-fucose synthase (GFS), and a fucosyl transferase (FT).

56. The microorganism of claim 55, wherein the transporter is a CDT-1 variant comprising an amino acid sequence having one or more amino acid replacements at positions corresponding to amino acid positions 91, 209, 256, 262, 335, 411 of SEQ ID NO:4.

57. The microorganism of claim 56, wherein the transporter comprises an amino acid replacement selected from the group consisting of 91A, 209S, 256V, 262Y, 262W, 335A, 411A and any combination thereof.

58. A method of producing 2′FL comprising:
providing a culture medium with at least one carbon source;
providing a microorganism of claim 55; and
culturing microorganism in the culture medium;
wherein a substantial portion of the 2′-FL is exported into the culture medium.

59. A 2′FL-containing product suitable for human or animal consumption comprising 2′-FL produced by the microorganism according to claim 55 and at least one additional consumable ingredient.

60. The product of claim 59, wherein the product is an infant formula, an infant food, a nutritional supplement or a prebiotic product.

61. A HMO-containing product suitable for human or animal consumption comprising an HMO produced by the microorganism according to claim 1 and at least one additional consumable ingredient.

62. The product of claim 61, wherein the product is an infant formula, an infant food, a nutritional supplement or a prebiotic product.

63. A microorganism for enhanced production of a human milk oligosaccharide (HMO) comprising (a) a heterologous CDT-1 transporter or a variant thereof; (b) at least one heterologous pathway gene for production of the HMO, wherein the microorganism is capable of producing and exporting the HMO and (c) a genetic modification that decreases the activity of SNF3 or RGT2 as compared to a parental microorganism without the genetic modification.
